(19) United States
(12) Patent Application Publication
(10) Pub. No.: US 2024/0398768 A1
(43) Pub. Date: Dec. 5, 2024

(54) METHODS OF TREATING SOLID TUMOR USING HETEROAROMATIC MACROCYCLIC ETHER COMPOUNDS

(71) Applicant: Nuvalent, Inc., Cambridge, MA (US)

(72) Inventors: Amit M. DESHPANDE, Medford, MA (US); Darlene NOCI, Burlington, MA (US); Henry Efrem PELISH, Newton, MA (US); James R. PORTER, Rowley, MA (US); John R. SOGLIA, Sherborn, MA (US); Anupong TANGPEERACHAIKUL, Cambridge, MA (US); Christopher Durant TURNER, Walpole, MA (US); Michael MEYERS, Hastings-on-Hudson, NY (US)

(21) Appl. No.: 18/697,204

(22) PCT Filed: Sep. 30, 2022
(86) PCT No.: PCT/US2022/077364

§ 371 Date: Mar. 29, 2024

Related U.S. Application Data
(60) Provisional application No. 63/251,536, filed on Oct. 1, 2021.
(60) Provisional application No. 63/357,309, filed on Jun. 30, 2022.

(51) Int. Cl. A61K 31/437; A61P 35/00

(52) U.S. Cl. CPC A61K 31/437; A61P 35/00

(57) ABSTRACT

Provided herein are methods of using a heteroaromatic macrocyclic ether compound (e.g., Compound 1), or a stereoisomer, or a mixture of stereoisomers thereof, or a pharmaceutically acceptable salt thereof, for treating, preventing or managing solid tumor.

This application claims the benefit of priority to U.S. Ser. No. 63/251,536 filed Oct. 1, 2021, and to U.S. Ser. No. 63/357,309 filed Jun. 30, 2022, each of which is incorporated herein by reference in its entirety.

### BACKGROUND

Receptor tyrosine kinases (RTKs) are cell surface enzymes that receive outside signals, such as whether to grow and divide, and transmit those signals in the cell through kinase activity. Many RTKs are proto-oncogenes; aberrant RTK activity can drive cell survival, growth and proliferation leading to cancer and related disorders. This aberrant kinase activity can be caused by mutations such as activating mutations in the kinase domain, gene rearrangements that result in fusion proteins containing the intact kinase domain, amplification and other means. RTK proto-oncogenes include ROS1, anaplastic lymphoma kinase (ALK), NTRK1 (encodes TRKA), NTRK2 (encodes TRKB), and NTRK3 (encodes TRKC).

ROS1 is an RTK proto-oncogene, with ROS1 rearrangements detected in non-small cell lung cancer (NSCLC), glioblastoma, inflammatory myofibroblastic tumor (IMT), cholangiocarcinoma, ovarian cancer, gastric cancer, colorectal cancer, angiosarcoma, and spitzoid melanoma. Oncogenic ROS1 gene fusions contain the kinase domain of ROS1 (3′ region) fused to the 5′ region of a variety of partner genes. Examples of ROS1 fusion partner genes observed in NSCLC include SLC34A2, CD74, TPM3, SDC4, EZR, LRIG3, KDELR2, CEP72, CLTL, CTNND2, GOPC, GPRC6A, LIMA1, LRIG3, MSN, MYO5C, OPRM1, SLC6A17 (putative), SLMAP, SRSF6, TFG, TMEM106B, TPD52L1, ZCCHC8 and CCDC6. Other fusion partners include CAPRIN1, CEP85L, CHCHD3, CLIP1 (putative), EEF1G, KIF21A (putative), KLC1, SART3, ST13 (putative), TRIM24 (putative), ERC1, FIP1L1, HLAA, KIAA1598, MYO5A, PPFIBP1, PWWP2A, FN1, YWHAE, CCDC30, NCOR2, NFKB2, APOB, PLG, RBP4, and GOLGB1.

NTRK1, NTRK2 and NTRK3 are RTK proto-oncogenes that encode TRK-family kinases, with NTRK1, NTRK2 and NTRK3 chromosomal rearrangements detected at low frequency in many cancers. For treatment of ROS1-positive or ALK-positive patients, however, TRK inhibition, particularly in the central nervous system (CNS), has been associated with adverse reactions, including dizziness/ataxia/gait disturbance, paraesthesia, weight gain and cognitive changes.

Existing agents used to treat oncogenic ROS1 and ALK have substantial deficiencies. These deficiencies may represent one or more of the following: associated TRK inhibition, limited CNS activity, and inadequate activity against resistance mutations. Treatment of ROS1-positive or ALK-positive patients accompanied by TRK inhibition is associated with adverse reactions, particularly in the CNS, including dizziness/ataxia/gait disturbance, paraesthesia, weight gain and cognitive changes. Additionally, there is a need for CNS-penetrant and TRK-sparing inhibitors of the wild type ROS1 kinase domain and ROS1 with acquired resistance mutations occurring either individually or in combination, including G2032R, D2033N, S1986F, S1986Y, L2026M, L1951R, E1935G, L1947R, G1971E, E1974K, L1982F, F2004C, F2004V, E2020K, C2060G, F2075V, V2089M, V2098I, G2101A, D2113N, D2113G, L2155S, L2032K, and L2086F. Likewise, there is a need for CNS-penetrant and TRK-sparing inhibitors of ALK with acquired resistance mutations. A variety of ALK drug resistance mutations, occurring either individually or in combination, have been reported, including G1202R, L1196M, G1269A, C1156Y, 11171T, 11171N, 11171S, F1174L, V1180L, S1206Y, E1210K, 1151Tins, F1174C, G1202del, D1203N, S1206Y, S1206C, L1152R, L1196Q, L1198P, L1198F, R1275Q, L1152P, C1156T, and F1245V.

### SUMMARY

Provided herein are methods of using a heteroaromatic macrocyclic ether compound (e.g., Compound 1), or a stereoisomer, or a mixture of stereoisomers thereof, or a pharmaceutically acceptable salt thereof, for treating, preventing or managing solid tumor.

In one embodiment, provided herein is a method of treating solid tumor, comprising administering to a patient in need thereof a therapeutically effective amount of Compound 1:

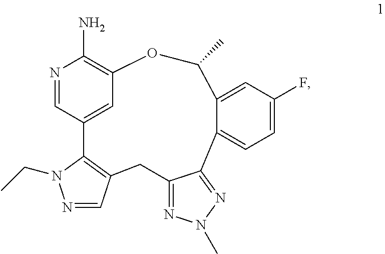 [SMILES: CCn1ncc2c1-c1cnc(N)c(c1)O[C@H](C)c1cc(F)ccc1-c1nn(C)nc1C2]

or a stereoisomer, or a mixture of stereoisomers thereof, or a pharmaceutically acceptable salt thereof. In certain embodiments, the compound is Compound 1.

In one embodiment, the solid tumor is advanced ROS1-positive solid tumor. In one embodiment, the solid tumor is locally advanced ROS1-positive solid tumor. In one embodiment, the solid tumor is advanced ROS1-positive non-small cell lung cancer (NSCLC).

In one embodiment, the solid tumor is metastatic ROS1-positive solid tumor. In one embodiment, the solid tumor is central nervous system (CNS) (e.g., brain) metastatic ROS1-positive solid tumor. In one embodiment, the solid tumor is metastatic ROS1-positive NSCLC. In one embodiment, the solid tumor is CNS (e.g., brain) metastatic ROS1-positive NSCLC.

In one embodiment, the patient is naïve to tyrosine kinase inhibitor (TKI) therapy. In one embodiment, the patient has been treated with one or more prior TKI therapies. In one embodiment, the patient has been treated with one prior ROS1 TKI therapy (e.g., crizotinib or entrectinib). In one embodiment, the patient has been treated with two or more prior ROS1 TKI therapies. In one embodiment, the patient's tumor does not have a known oncogenic driver alteration other than ROS1.

In one embodiment, the compound is administered at an amount of from about 5 mg to about 400 mg once (QD) or twice (BID) daily. In one embodiment, the compound is administered at an amount of from about 5 mg to about 200 mg (by weight of Compound 1) once (QD) or twice (BID) daily. In one embodiment, the compound is administered to a patient with an empty stomach (e.g., at least 1 hour before and no sooner than 2 hours after ingestion of food and/or beverages other than water). In one embodiment, the compound is administered to a patient following ingestion of food and/or beverages. In one embodiment, the patient is not taking any one of strong inhibitors of CYP3A4, strong inducers of CYP3A4, sensitive substrates of CYP3A4, substrates of P-gp/multidrug resistance protein (MDR1), substrates of BCRP/breast cancer resistance protein (ABCG2), substrates of MATE1, or gastric acid reducing agents. In one embodiment, the patient is taking any one of strong inhibitors of CYP3A4, strong inducers of CYP3A4, sensitive substrates of CYP3A4, substrates of P-gp/multidrug resistance protein (MDR1), substrates of BCRP/breast cancer resistance protein (ABCG2), substrates of MATE1, or gastric acid reducing agents. In one embodiment, the compound is administered in the absence of a strong inhibitor of CYP3A4, a strong inducers of CYP3A4, or a sensitive substrate of CYP3A4. In one embodiment, the compound is administered in the presence of a strong inhibitor of CYP3A4, a strong inducer of CYP3A4, or a sensitive substrate of CYP3A4. In one embodiment, the compound is administered in the absence of a strong inhibitor of CYP3A4. In one embodiment, the compound is administered in the absence of a strong inducer of CYP3A4. In one embodiment, the compound is administered in the absence of a sensitive substrate of CYP3A4. In one embodiment, the compound is administered in the absence of a substrate of P-gp/multidrug resistance protein 1 (MDR1), a substrate of BCRP/breast cancer resistance protein (ABCG2) or MATE1. In one embodiment, the compound is administered in the presence of a substrate of P-gp/multidrug resistance protein 1 (MDR1), a substrate of BCRP/breast cancer resistance protein (ABCG2) or MATE1. In one embodiment, the compound is administered in the absence of a gastric acid reducing agent (e.g., proton pump inhibitors, e.g., lansoprazole). In one embodiment, the compound is administered in the presence of a gastric acid reducing agent (e.g., proton pump inhibitors, e.g., lansoprazole). In one embodiment, the compound is administered in the absence of a strong inducer of CYP3A4. In one embodiment, the compound is administered in the presence of a strong inducer of CYP3A4.

In one embodiment, the compound is administered in the absence of a sensitive substrate of CYP3A4. In one embodiment, the compound and a sensitive substrate of CYP3A4 are not administered concomitantly. In one embodiment, the sensitive substrates of CYP3A4 comprise one or more of buspirone, everolimus, lovastatin, midazolam, simvastatin, triazolam, maraviroc, conivaptan, and darifenacin.

### BRIEF DESCRIPTION OF DRAWINGS

FIG. 1 shows phase 1 and phase 2 study design.

FIG. 2A, FIG. 2B, and FIG. 2C show Compound 1 induced regression in Lu01-0414, CTG-0848, and CTG-2532 Patient-Derived Xenograft Models (PDX), respectively. The activity of Compound 1 in various ROS1 PDX models subcutaneously implanted in Balb/c mice. Vehicle is 20% HP-β-CD (hydroxypropyl-beta-cyclodextrin). Average±SEM plotted. No significant changes in body weight were observed (data not shown). Compound 1 induced regression irrespective of fusion partner (SDC4 or CD74).

FIG. 3 shows ROS1 fusions signal through the PI3K and the MAP kinase pathways to effect proliferation and other transcriptional changes.

FIG. 4A shows the representative western blot of Lu01-0414 PDX treated with Compound 1 (BID×5 PO) revealed suppression of ROS1-dependent cell signaling through reduced levels of pROS1, ROS1, pAKT, and pERK. Pharmacokinetic analysis indicated dose-dependent differences in Compound 1 unbound plasma concentrations. In this model, 0.04 mg/kg achieved stasis and 5 mg/kg achieved regression. FIG. 4B, FIG. 4C, and FIG. 4D: Western blot quantitation of three PDX models treated with Compound 1 (BID×5 PO) confirmed reduced levels of ROS1, pROS1, and pERK. Y-axis denotes fold-change normalized to vehicle treatment, indicated with unfilled black square and horizontal grey line. Mean±SEM plotted.

FIG. 5A, FIG. 5B, and FIG. 5C show Compound 1 inhibited ROS1 signaling in tumors by quantitative digital image analysis of immunohistochemical stains. Mean±SEM plotted. Effects on pERK, ERK and pAKT were model-dependent.

FIG. 6A, FIG. 6B, and FIG. 6C show Compound 1 reduced expression levels of MAP kinase pathway genes in tumors. Gene expression profiling of tumor samples with the NanoString 770 gene nCounter PanCancer Pathways panel. Expression levels of MAP kinase pathway genes DUSP6, FOS, ILIR1, and SPRY4 were dose-dependently reduced in the tumors upon Compound 1 treatment. The Y-axis denotes relative expression normalized to vehicle treatment, indicated with black dots and horizontal grey line. Mean±SEM plotted.

FIG. 7 shows an additional phase 1 study design.

FIG. 8A shows representative computed tomography (CT) images demonstrating a confirmed partial response (PR) to treatment with Compound 1 in a patient with CD74-ROS1 fusion-positive lung adenocarcinoma with a ROS1 G2032R resistance mutation after prior crizotinib, lorlatinib, and chemotherapy. Arrows indicate left lung nodules that decreased in size over the course of treatment.

FIG. 8B shows representative CT (upper panel) and magnetic resonance imaging (lower panel) images demonstrating a confirmed PR to treatment with Compound 1 in a patient with EZR-ROS1 fusion-positive lung adenocarcinoma with a ROS1 G2032R resistance mutation after prior entrectinib, repotrectinib, and chemotherapy. Arrows in the upper panel indicate segment 5/6 and 3 liver metastases with continuous regression over the course of treatment. Arrow in the lower panel indicates a right occipital lobe metastasis that decreased in size at week 4 and became barely appreciable at week 16.

FIG. 8C shows representative computed tomography images demonstrating a confirmed partial response to Compound 1 in a patient with EZR-ROS1 fusion-positive lung adenocarcinoma without known ROS1 resistance mutations after prior crizotinib. Arrows indicate left pleural-based lung nodules that decreased in size at week 4 and became barely appreciable at week 24.

FIG. 9 shows that Compound 1 inhibits signaling through the MAPK and PI3K/AKT pathways and induces apoptosis in PDX tumors harboring SDC4-ROS1.

FIG. 10A is a representative X-ray powder diffraction (XRPD) pattern of Form 1 of free base of Compound 1. FIG. 10B is a representative differential scanning calorimetry (DSC) thermogram of Form 1 of free base of Compound 1.

FIG. 11 shows ctDNA data indicating that Compound 1 was active on both ROS1 fusion and ROS1 G2032R solvent front mutation at all dose levels tested.

FIG. 12 shows radiographic tumor regression for patients dosed with Compound 1. PD=progressive disease; PR=confirmed partial response; QD=once daily; SD=stable disease; TKI=tyrosine kinase inhibitor. ^{a}Single-timepoint PR not confirmed. ^{b}Ongoing partial responses pending confirmation. ^{c}Best response PR due to residual nontarget disease. ^{d}Additional prior ROS1 TKI was ceritinib or cabozantinib. ^{e}Including immunotherapy, bevacizumab, and investigational therapy.

FIG. 13 shows sustained duration of treatment for response-evaluable patients with NSCLC administered Compound 1. PD=progressive disease; PR=partial response; TKI=tyrosine kinase inhibitor.

FIG. 14 shows reduction in tumor burden in patients with ROS1 G2032R by Compound 1 (left panel) and reduction in ROS1 G2032R allele by Compound 1 (right panel). ^{b} Bar represents week 2 result; week 8 results are pending.

FIG. 15 shows radiographic images indicating intracranial response in a 65-year-old female with CD74-ROS1 fusion NSCLC, previously treated with chemotherapy, crizotinib, and lorlatinib with CNS progression and no known ROS1 resistance mutations.

FIG. 16 shows radiographic images indicating intracranial and extracranial activity in TKI-refractory ROS1 G2032R+NSCLC in a patient diagnosed with EZR-ROS1 fusion NSCLC.

FIG. 17 shows EZR-ROS1 (left y-axis) and ROS1 G2032R (right y-axis) variant allele frequencies in the plasma of a 65-year-old patient with stage IV lung adenocarcinoma and multiple brain metastases had EZR-ROS1 identified by plasma testing, before treatment (C1D1) and on-treatment (C1D15 and C3D1), as determined by the Guardant 360 assay.

FIG. 18A, FIG. 18B, and FIG. 18C show the unbound plasma concentrations of Compound 1 of three patients on the treatment cycle 1 day 15 over a 24-hour period (see the results section of Example 5). The dashed line indicates the minimally efficacious concentration threshold of 5.3 ng/mL=13 nM, which is based on the efficacious threshold in the murine CTG-2532 CD74-ROS1 G2032R PDX study.

FIG. 19 shows that compound 1 exposure in clinical trial exceeded the target levels that provide regression in preclinical models (see Example 5). Compound 1 demonstrated favorable pharmacokinetics including the low intra-cohort patient PK variability, exposure increasing with increasing dose level, half-life approximately 20h supports QD dosing. Data as of Sep. 13, 2022, for patients treated by Sep. 1, 2022. Pharmacokinetic data for 125 mg QD cohort are not shown due to immaturity. QD=once a day. ^{a,b}Based on tumor regression in in vivo models bearing SDC4-ROS1 and CD74-ROS1 G2032R, respectively. ^{c,d}Values for a and b divided by predicted human CNS Kp, respectively.

FIG. 20 shows phase 1 population in Example 5 has the heavily pretreated ROS1 positive solid tumors according to the data of Sep. 13, 2022, for patients treated by Sep. 1, 2022. All data shown as n (%) unless otherwise specified. CNS, central nervous system; ECOG PS, Eastern Cooperative Oncology Group performance status; NSCLC, non-small cell lung cancer; RECIST 1.1, Response Evaluation Criteria in Solid Tumours version 1.1; TKI, tyrosine kinase inhibitor. ^{a}Includes patients with untreated CNS lesions. ^{b}Excludes therapies given for early-stage disease. ^{c}Categories are not mutually exclusive.

FIG. 21 shows the treatment-related adverse events (TRAEs) in >1 patient in the phase 1 trial (Example 5) according to the data of Sep. 13, 2022, for patients treated by Sep. 1, 2022. The safety profile of Compound 1 is favorable and consistent with the highly ROS1-selective, TRK-sparing Compound 1. AE, adverse event; ALT, Alanine aminotransferase; AST, Aspartate aminotransferase; DLT, dose-limiting toxicity; SAE, serious adverse event. ª Including oedema and oedema peripheral.

### DETAILED DESCRIPTION

### Definitions

Unless defined otherwise, all technical and scientific terms used herein have the meaning commonly understood by a person skilled in the art of the present disclosure. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

In some embodiments, chemical structures are disclosed with a corresponding chemical name. In case of conflict, the chemical structure controls the meaning, rather than the name.

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments of the invention.

The term “consisting of” means that a subject-matter has at least 90%, 95%, 97%, 98% or 99% of the stated features or components of which it consists. In another embodiment the term “consisting of” excludes from the scope of any succeeding recitation any other features or components, excepting those that are not essential to the technical effect to be achieved.

Unless specifically stated or obvious from context, as used herein, the term “or” is understood to be inclusive. Unless specifically stated or obvious from context otherwise, as used herein, the terms “a”, “an”, and “the” are understood to be singular or plural. For example, when a compound provided herein is administered to “a patient”, it includes administering the compound to an individual patient or a patient population.

As used herein and unless otherwise specified, “stereoisomers” refer to the various stereoisomeric forms of a compound that comprises one or more asymmetric centers or stereohindrance in the structure. In some embodiments, a stereoisomer is an enantiomer, a mixture of enantiomers, an atropisomer, or a tautomer thereof. For example, the compounds described herein can be in the form of an individual enantiomer, diastereomer or geometric isomer (e.g. an atropisomer), or can be in the form of a mixture of stereoisomers, including racemic mixtures and mixtures enriched in one or more stereoisomer. In some embodiments, compounds provided herein may be atropisomers. In certain embodiments, atropisomers are stereoisomers arising because of hindered rotation about a single bond, where energy differences due to steric strain or other contributors create a barrier to rotation that is high enough to allow for isolation of individual conformers. Stereoisomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H. Tables of Resolving Agents and Optical Resolutions p. 268 (E. L. Eliel, Ed., Univ. of Notre Dame Press, Notre Dame, IN 1972). The invention additionally encompasses compounds as individual isomers substantially free of other isomers, and alternatively, as mixtures of various isomers.

In certain embodiments, compounds provided herein may be racemic. In certain embodiments, compounds provided herein may be enriched in one enantiomer. For example, a compound provided herein may have greater than about 30% ee, about 40% ee, about 50% ee, about 60% ee, about 70% ee, about 80% ee, about 90% ee, or even about 95% or greater ee. In certain embodiments, compounds provided herein may have more than one stereocenter. In certain such embodiments, compounds provided herein may be enriched in one or more diastereomer. For example, a compound provided herein may have greater than about 30% de, about 40% de, about 50% de, about 60% de, about 70% de, about 80% de, about 90% de, or even about 95% or greater de.

In certain embodiments, the therapeutic preparation may be enriched to provide predominantly one enantiomer of a compound. An enantiomerically enriched mixture may comprise, for example, at least about 60 mol percent of one enantiomer, or more particularly at least about 75, about 90, about 95, or even about 99 mol percent. In certain embodiments, the compound enriched in one enantiomer is substantially free of the other enantiomer, wherein substantially free means that the substance in question makes up less than about 10%, or less than about 5%, or less than about 4%, or less than about 3%, or less than about 2%, or less than about 1% as compared to the amount of the other enantiomer, e.g., in the composition or compound mixture. For example, if a composition or compound mixture contains about 98 grams of a first enantiomer and about 2 grams of a second enantiomer, it would be said to contain about 98 mol percent of the first enantiomer and only about 2% of the second enantiomer.

In certain embodiments, the therapeutic preparation may be enriched to provide predominantly one diastereomer of a compound. A diastereomerically enriched mixture may comprise, for example, at least about 60 mol percent of one diastereomer, or more particularly at least about 75, about 90, about 95, or even about 99 mol percent.

In some embodiments, a moiety in a compound exists as a mixture of tautomers. A “tautomer” is a structural isomer of a moiety or a compound that readily interconverts with another structural isomer. For example, a pyrazole ring has two tautomers:

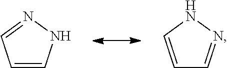 [SMILES: c1cn[nH]c1 ; c1cn[nH]c1]

which differ in the positions of the pi-bonds and a hydrogen atom. Unless explicitly stated otherwise, a drawing of one tautomer of a moiety or a compound encompasses all of the possible tautomers.

The term “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys. In certain embodiments, the subject is a human. In certain embodiments, the subject is a human adult at least of 40 years old. In certain embodiments, the subject is a human adult at least of 50 years old. In certain embodiments, the subject is a human adult at least of 60 years old. In certain embodiments, the subject is a human adult at least of 70 years old. In certain embodiments, the subject is a human adult at least of 18 years old or at least of 12 years old. As used herein and unless otherwise specified, a human subject to which administration of a therapeutic (e.g., a compound as described herein) is contemplated in order to treat, prevent or manage a disease, disorder, or condition, or symptoms thereof, is also called a “patient”.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample. These effects are also called “prophylactic” effects. Thus, as used herein and unless otherwise specified, the terms “prevention” and “preventing” refer to an approach for obtaining beneficial or desired results including, but not limited, to prophylactic benefit. For prophylactic benefit, a therapeutic can be administered to a patient at risk of developing a particular disease, or to a patient reporting one or more of the physiological symptoms of a disease, even though a diagnosis of this disease may not have been made. In one embodiment, a therapeutic is administered prior to clinical manifestation of the unwanted condition (e.g., disease or other unwanted state of the subject) for prophylactic benefit (e.g., it protects the subject against developing the unwanted condition).

As used herein and unless otherwise specified, the terms “treatment” and “treating” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment. In one embodiment, “treatment” comprises administration of a therapeutic after manifestation of the unwanted condition (i.e., it is intended to diminish, ameliorate, or stabilize the existing unwanted condition or side effects thereof).

As used herein and unless otherwise indicated, the term “managing” encompasses preventing the recurrence of the particular disease or disorder in a patient who had suffered from it, lengthening the time a patient who had suffered from the disease or disorder remains in remission, reducing mortality rates of the patients, and/or maintaining a reduction in severity or avoidance of a symptom associated with the disease or condition being managed.

An “effective amount”, as used herein, refers to an amount that is sufficient to achieve a desired biological effect. A “therapeutically effective amount”, as used herein, refers to an amount that is sufficient to achieve a desired therapeutic effect. For example, a therapeutically effective amount can refer to an amount that is sufficient to improve at least one sign or symptom of cancer.

A “response” to a method of treatment can include a decrease in or amelioration of negative symptoms, a decrease in the progression of a disease or symptoms thereof, an increase in beneficial symptoms or clinical outcomes, a lessening of side effects, stabilization of disease, partial or complete remedy of disease, among others.

As used herein and unless otherwise indicated, the term “relapsed” refers to a disorder, disease, or condition that responded to prior treatment (e.g., achieved a complete response) then had progression. The prior treatment can include one or more lines of therapy.

As used herein and unless otherwise indicated, the term “refractory” refers to a disorder, disease, or condition that has not responded to prior treatment that can include one or more lines of therapy.

As used herein, and unless otherwise specified, the terms “about” and “approximately,” when used in connection with doses, amounts, or weight percents of ingredients of a composition or a dosage form, mean a dose, amount, or weight percent that is recognized by one of ordinary skill in the art to provide a pharmacological effect equivalent to that obtained from the specified dose, amount, or weight percent. In certain embodiments, the terms “about” and “approximately,” when used in this context, contemplate a dose, amount, or weight percent within 30%, within 20%, within 15%, within 10%, or within 5%, of the specified dose, amount, or weight percent.

The term “between” includes the endpoint numbers on both limits of the range. For example, the range described by “between 3 and 5” is inclusive of the numbers “3” and “5”.

As used herein and unless otherwise specified, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19. In certain embodiments, pharmaceutically acceptable salts include, but are not limited to, alkyl, dialkyl, trialkyl or tetra-alkyl ammonium salts. In certain embodiments, pharmaceutically acceptable salts include, but are not limited to, L-arginine, benenthamine, benzathine, betaine, calcium hydroxide, choline, deanol, diethanolamine, diethylamine, 2-(diethylamino) ethanol, ethanolamine, ethylenediamine, N-methylglucamine, hydrabamine, 1H-imidazole, lithium, L-lysine, magnesium, 4-(2-hydroxyethyl) morpholine, piperazine, potassium, 1-(2-hydroxyethyl) pyrrolidine, sodium, triethanolamine, tromethamine, and zinc salts. In certain embodiments, pharmaceutically acceptable salts include, but are not limited to, Na, Ca, K, Mg, Zn or other metal salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Pharmaceutically acceptable anionic salts include, but are not limited to, acetate, aspartate, benzenesulfonate, benzoate, besylate, bicarbonate, bitartrate, bromide, camsylate, carbonate, chloride, citrate, decanoate, edetate, esylate, fumarate, gluceptate, gluconate, glutamate, glycolate, hexanoate, hydroxynaphthoate, iodide, isethionate, lactate, lactobionate, malate, maleate, mandelate, mesylate, methylsulfate, mucate, napsylate, nitrate, octanoate, oleate, pamoate, pantothenate, phosphate, polygalacturonate, propionate, salicylate, stearate, acetate, succinate, sulfate, tartrate, teoclate, and tosylate.

### Compounds

In one embodiment, the compound used in the methods provided herein is a compound of the following formula, also referred to as “Compound 1”:

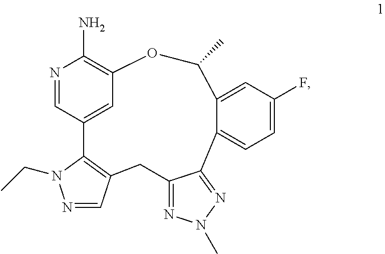 [SMILES: CCn1ncc2c1-c1cnc(N)c(c1)O[C@H](C)c1cc(F)ccc1-c1nn(C)nc1C2]

or a stereoisomer, or a mixture of stereoisomers thereof, or a pharmaceutically acceptable salt thereof. Compound 1 has the chemical name of (19R)-3-ethyl-16-fluoro-10,19-dimethyl-20-oxa-3,4,9,10,11,23-hexaazapentacyclo [19.3.1.0²,⁶.0⁸,¹².0¹³,¹⁸]pentacosa-1 (24),2 (6),4,8,11,13,15,17,21 (25),22-decaen-22-amine, and is described in International Application No. PCT/US2021/030842, the entirety of which is incorporated herein by reference. As used herein, “Compound 1,” “Compound 1 free base,” “Compound 1 (free base),” and “free base Compound 1” are used interchangeably.

It is to be understood that, unless otherwise specified, when the compound as described herein is provided as a pharmaceutically acceptable salt thereof, that the weight amount refers to the portion exclusive of the salt portion (i.e. as the Compound 1 free base).

In one embodiment, Compound 1 (free base) is used in the methods provided herein. In one embodiment, a tautomer of Compound 1 is used in the methods provided herein. In one embodiment, Compound 1 substantially free of the(S)-enantiomer is used in the methods provided herein. As used herein, “substantially free” means the(S)-enantiomer is present in less than about 10 wt % of Compound 1 (e.g. less than about 5 wt % or less than about 0.05 wt % of Compound 1). In one embodiment, Compound 1 substantially pure with the enantiomeric purity of at least about 98% (e.g. 99% or 99.5%). In one embodiment, a pharmaceutically acceptable salt of Compound 1 is used in the methods provided herein. In one embodiment, the pharmaceutically acceptable salt is a besylate salt. In one embodiment, the pharmaceutically acceptable salt is a phosphate salt.

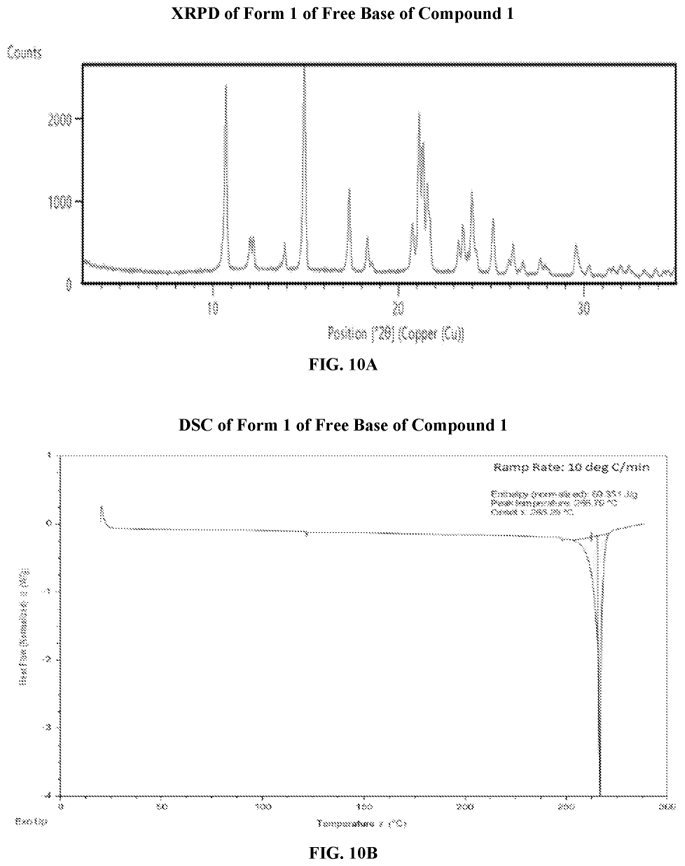

In one embodiment, the compound used in the methods provided herein is a solid form of Compound 1. In one embodiment, the solid form is a Form 1 of Compound 1. A representative XRPD pattern of Form 1 of Compound 1 is provided in FIG. 10A.

In one embodiment, the solid form of Compound 1 is characterized by 1, 2, 3, 4, 5, 6, 7, 8, 9, 10, 11, 12, 13, 14, 15, 16, 17, 18, 19, 20, 21, 22 or all of the XRPD peaks located at approximately the following positions (e.g., degrees 2θ±0.2) when measured using Cu Kα radiation: 10.7, 12.0, 12.2, 13.9, 15.0, 17.4, 18.4, 18.6, 20.8, 21.2, 21.3, 21.6, 21.7, 23.3, 23.5, 24.0, 25.2, 26.0, 26.2, 26.7, 27.7, 28.0, and 29.6° 2θ. In one embodiment, the solid form is characterized by 3 of the peaks. In one embodiment, the solid form is characterized by 5 of the peaks. In one embodiment, the solid form is characterized by 7 of the peaks. In one embodiment, the solid form is characterized by 9 of the peaks. In one embodiment, the solid form is characterized by 11 of the peaks. In one embodiment, the solid form is characterized by all of the peaks.

In one embodiment, the solid form (e.g., a crystalline form) of Compound 1 is characterized by an XRPD pattern, when measured using Cu Kα radiation, comprising at least three peaks selected from the group consisting of approximately (e.g., ±0.2°)10.7, 15.0, 17.4, 20.8, 21.2, 21.3, 21.6, 24.0, and 25.2° 2θ. In one embodiment, the solid form is characterized by an XRPD pattern comprising at least four peaks selected from the group consisting of approximately (e.g., ±0.2°)10.7, 15.0, 17.4, 20.8, 21.2, 21.3, 21.6, 24.0, and 25.2° 2θ. In one embodiment, the solid form is characterized by an XRPD pattern comprising at least five peaks selected from the group consisting of approximately (e.g., ±0.2°)10.7, 15.0, 17.4, 20.8, 21.2, 21.3, 21.6, 24.0, and 25.2° 2θ.

In one embodiment, the solid form (e.g., a crystalline form) of Compound 1 is characterized by an XRPD pattern, when measured using Cu Kα radiation, comprising at least three peaks selected from the group consisting of approximately (e.g., ±0.2°)10.7, 12.0, 12.2, 13.9, 15.0, 17.4, 18.4, 20.8, 21.2, 21.3, 21.6, 24.0, and 25.2° 2θ. In one embodiment, the solid form is characterized by an XRPD pattern comprising at least four peaks selected from the group consisting of approximately (e.g., ±0.2°)10.7, 12.0, 12.2, 13.9, 15.0, 17.4, 18.4, 20.8, 21.2, 21.3, 21.6, 24.0, and 25.2° 2θ. In one embodiment, the solid form is characterized by an XRPD pattern comprising at least five peaks selected from the group consisting of approximately (e.g., ±0.2°)10.7, 12.0, 12.2, 13.9, 15.0, 17.4, 18.4, 20.8, 21.2, 21.3, 21.6, 24.0, and 25.2° 2θ.

In one embodiment, the solid form (e.g., a crystalline form) of Compound 1 is characterized by an XRPD pattern comprising peaks at approximately (e.g., ±0.2°)10.7, 15.0, and 21.2° 2θ. In one embodiment, the XRPD pattern further comprises peaks at approximately (e.g., ±0.2°)17.4 and 21.3° 2θ. In one embodiment, the XRPD pattern further comprises peaks at approximately (e.g., ±0.2°)12.0, 12.2, and 13.9° 2θ. In another embodiment, the XRPD pattern further comprises peaks at approximately (e.g., ±0.2°)21.6 and 24.0° 2θ. In one embodiment, the XRPD pattern comprises peaks at approximately (e.g., ±0.2°)10.7, 15.0, 17.4, 20.8, 21.2, 21.3, 21.6, 24.0 and 25.2° 2θ. In one embodiment, the XRPD pattern comprises peaks at approximately (e.g., ±0.2°)10.7, 12.0, 12.2, 13.9, 15.0, 17.4, 18.4, 20.8, 21.2, 21.3, 21.6, 24.0 and 25.2° 2θ.

In one embodiment, the solid form is characterized by an XRPD pattern that matches the XRPD pattern depicted in FIG. 10A.

In one embodiment, an XRPD pattern described herein is obtained using Cu Kα radiation. In one embodiment, the XRPD pattern is measured by XRPD using Cu Kα radiation comprising Kai radiation having a wavelength of 1.5406 Å and Kα₂ radiation having a wavelength of 1.5444 Å, wherein the Kai: Kα₂ ratio is 0.5.

A representative DSC thermogram of Form 1 is provided in FIG. 10B. In one embodiment, the solid form exhibits, as characterized by DSC, a thermal event (endothermic) with an onset temperature of about 265° C. (e.g. ±2°). In one embodiment, the thermal event also has a peak temperature of about 267° C. (e.g. ±2°). In one embodiment, without being bound by a particular theory, the thermal event corresponds to melting. In one embodiment, the solid form is characterized by a DSC thermogram that matches the DSC thermogram depicted in FIG. 10B. In one embodiment, the DSC thermogram is as measured by DSC using a scanning rate of about 10° C./minute.

In another embodiment, the solid form has a melting point at about 270° C. (e.g. ±2°).

In one embodiment, the solid form (e.g., a crystalline form) of Compound 1 has approximately unit cell dimensions of: a=8.4 Å, b=8.4 Å, c=14.9 Å, a=90°, B=106°, and Y=90°. In one embodiment, the solid form has approximately unit cell dimensions of: a=8.43 Å, b=8.44 Å, c=14.91 Å, a=90°, B=105.6°, and γ=90°. In one embodiment, the solid form has approximately unit cell dimensions of: a=8.431 Å, b=8.441 Å, c=14.914 Å, a=90°, β=105.55°, and γ=90°. In one embodiment, Form 1 has a unit cell of a space group of P2₁. In one embodiment, the solid form has a volume of about 1022.7 Å³/cell. In one embodiment, Form 1 has a Z value of 2. In one embodiment, Form 1 has a density of about 1.362 g/cm³.

In one embodiment, the(S)-enantiomer of Compound 1 is used in the methods provided herein. In one embodiment, a tautomer of the(S)-enantiomer of Compound 1 is used in the methods provided herein. In one embodiment, a pharmaceutically acceptable salt of the(S)-enantiomer of Compound 1 is used in the methods provided herein.

In one embodiment, the racemic mixture of Compound 1 is used in the methods provided herein. In one embodiment, a tautomer of the racemic mixture of Compound 1 is used in the methods provided herein. In one embodiment, a pharmaceutically acceptable salt of the racemic mixture of Compound 1 is used in the methods provided herein.

### Methods of Use

In one embodiment, provided herein are methods of treating cancer comprising administering a heteroaromatic macrocyclic ether compound, such as Compound 1, or a stereoisomer, or a mixture of stereoisomers thereof, or a pharmaceutically acceptable salt thereof.

In one embodiment, provided herein are methods of using a heteroaromatic macrocyclic ether compound (e.g., Compound 1), or a stereoisomer, or a mixture of stereoisomers thereof, or a pharmaceutically acceptable salt thereof, for treating, preventing or managing solid tumor.

In one embodiment, provided herein is a method of treating a patient with solid tumor, comprising administering to said patient a therapeutically effective amount of Compound 1:

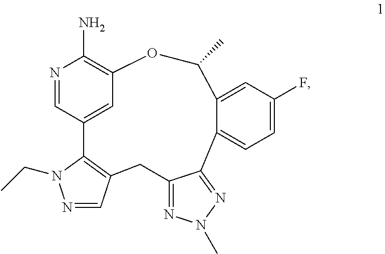 [SMILES: CCn1ncc2c1-c1cnc(N)c(c1)O[C@H](C)c1cc(F)ccc1-c1nn(C)nc1C2]

or a stereoisomer, or a mixture of stereoisomers thereof, or a pharmaceutically acceptable salt thereof.

In one embodiment, the cancer is anaplastic large cell lymphoma (ALCL), atypical meningioma, breast cancer, cholangiocarcinoma, gastric cancer, glioblastoma, inflammatory myofibroblastic tumor (IMT), inflammatory hepatocellular adenoma (HCA), melanoma, pancreatic cancer, papillary thyroid carcinoma, salivary gland carcinoma, serous ovarian carcinoma, or spitzoid neoplasm.

In one embodiment, the solid tumor is advanced solid tumor. In one embodiment, the solid tumor is locally advanced solid tumor. In one embodiment, the advanced solid tumor is relapsed after, refractory to, or resistant to the prior treatment by a tyrosine kinase inhibitor (TKI). In one embodiment, the solid tumor is non-small cell lung cancer (NSCLC). In one embodiment, the solid tumor is ROS1 positive (e.g. ROS1 fusion) NSCLC. In one embodiment, the solid tumor is ALK positive (e.g. ALK fusion) NSCLC. In one embodiment, the solid tumor is LTK positive (e.g. LTK fusion) NSCLC. In one embodiment, the solid tumor is advanced NSCLC. In one embodiment, the solid tumor is locally advanced NSCLC. In one embodiment, the solid tumor is metastatic. In one embodiment, the solid tumor is metastatic to the CNS (also referred to herein as “CNS metastatic” or “metastatic CNS”). In one embodiment, the solid tumor is metastatic NSCLC. In one embodiment, the solid tumor is NSCLC metastatic to the CNS. As used herein and unless otherwise specified, “advanced tumor” refers to a tumor that cannot be cured or grows beyond the initial site of origin, either locally advanced or metastatic.

In one embodiment, the solid tumor (or cancer) is ROS1 positive. In one embodiment, the solid tumor is ROS1 positive NSCLC. In one embodiment, the solid tumor is advanced ROS1 positive solid tumor. In one embodiment, the solid tumor is locally advanced ROS1 positive solid tumor. In one embodiment, the solid tumor is advanced ROS1 positive NSCLC. In one embodiment, the solid tumor is locally advanced ROS1 positive NSCLC. In one embodiment, the solid tumor is metastatic ROS1 positive solid tumor. In one embodiment, the solid tumor is CNS metastatic ROS1 positive solid tumor. In one embodiment, the solid tumor is metastatic ROS1 positive NSCLC. In one embodiment, the solid tumor is CNS metastatic ROS1 positive NSCLC. In one embodiment, the solid tumor (or cancer) has a ROS1 mutation. In one embodiment, the ROS1 mutation is G2032R. In one embodiment, the ROS1 mutation comprise G2032R and one or more of S1986F, S1986Y, F2004C, F2004V, L2026M, D2033N, or G2101A. In one embodiment, the solid tumor (or cancer) has a ROS1 fusion.

In one embodiment, the solid tumor (or cancer) is ALK positive. As used herein and unless otherwise specified, an “ALK positive” (ALK+) cancer, disease, or disorder refers to a cancer, disease, or disorder characterized by inappropriately high expression of an ALK gene and/or the presence of a mutation in an ALK gene and/or the presence of a partially deleted ALK protein. In one embodiment, the solid tumor is ALK positive NSCLC. In one embodiment, the solid tumor is advanced ALK positive solid tumor. In one embodiment, the solid tumor is locally advanced ALK positive solid tumor. In one embodiment, the solid tumor is advanced ALK positive NSCLC. In one embodiment, the solid tumor is locally advanced ALK positive NSCLC. In one embodiment, the solid tumor is metastatic ALK positive solid tumor. In one embodiment, the solid tumor is CNS metastatic ALK positive solid tumor. In one embodiment, the solid tumor is metastatic ALK positive NSCLC. In one embodiment, the solid tumor is CNS metastatic ALK positive NSCLC. In one embodiment, the solid tumor (or cancer) has an ALK mutation.

In one embodiment, the ALK mutation comprises one or more ALK rearrangements (in one embodiment, one rearrangement). In one embodiment, the ALK mutation comprises one or more ALK fusions (in one embodiment, one fusion). In some embodiments, cancers treated by methods of the present disclosure include ALK fusions. In one embodiment, the ALK fusion is with one of the fusion partners described in Ou et al., JTO Clinical and Research Reports, 1 (1): 1-10, the entirety of which is incorporated herein by reference. In one embodiment, the ALK fusion is with one of the fusion partners selected from the group consisting of EML4, TFG, KIF5B, KLC1, STRN, HIP1, TPR, BIRC6, DCTN1, SQSTM1, SOCS5, SEC31A, CLTC, PRKAR1A, PPM1B, EIF2AK3, CRIM1, CEBPZ, PICALM, CLIP1, BCL11A, GCC2, LMO7, PHACTR1, CMTR1, VIT, DYSF, ITGAV, PLEKHA7, CUX1, VKORC1L1, FBXO36, SPTBN1, EML6, FBXO11, CLIP4, CAMKMT, NCOA1, MYT1L, SRBD1, SRD5A2, NYAP2, MPRIP, ADAM17, ALK, LPIN1, WDPCP, CEP55, ERC1, SLC16A7, TNIP2, ATAD2B, SLMAP, FBN1, SWAP70, TCF12, TRIM66, WNK3, AKAP8L, SPECC1L, PRKCB, CDK15, LCLAT1, YAP1, PLEKHM2, DCHS1, PPFIBP1, ATP13A4, C12orf75, EPAS1, FAM179A, FUT8, LIMD1, LINC00327, LOC349160, LYPD1, RBM20, TACR1, TANC1, TTC27, TUBBB, SMPD4, SORCS1, LINC00211, SOS1, C9orf3, CYBRD1, MTA3, THADA, TSPYL6, WDR37, and PLEKHH2. In one embodiment, the ALK fusion is with one of the fusion partners selected from the group consisting of EML4, TMP1, WDCP, GTF2IRD1, TPM3, TPM4, CLTC, LMNA, PRKAR1A, RANBP2, TFG, FN1, KLC1, VCL, STRN, HIP1, NPM1, DCTN1, SQSTM1, TPR, CRIM1, PTPN3, FBXO36, ATIC and KIF5B. In one embodiment, the ALK mutation is EML4-ALK, a fusion between the echinoderm microtubule-associated protein-like 4 (EML4) gene and the ALK tyrosine kinase domain. There are many variants of EML4-ALK that differ by breakpoint junctions, with variant 1 (v1) and variant 3 (v3) being the most prevalent clinically. In one embodiment, the ALK mutation is NPM1-ALK. In one embodiment, the ALK mutation is STRN-ALK.

In one embodiment, the solid tumor (or cancer) is leukocyte receptor tyrosine kinase (LTK) positive. As used herein and unless otherwise specified, an “LTK positive” (LTK+) cancer, disease, or disorder refers to a cancer, disease, or disorder characterized by inappropriately high expression of an LTK gene and/or the presence of a mutation in an LTK gene, including LTK gene rearrangements resulting in LTK fusion proteins. In one embodiment, the solid tumor is LTK positive breast invasive ductal carcinoma, prostate adenocarcinoma, pancreatic adenocarcinoma, adenocarcinoma of unknown primary, or bladder urothelial carcinoma. In one embodiment, the cancer is LTK positive leukemia. In one embodiment, the solid tumor is LTK positive lung cancer. In one embodiment, the solid tumor is LTK positive NSCLC. In one embodiment, the solid tumor (or cancer) has an LTK mutation. In one embodiment, the LTK mutation is G269A, F2181, N257T, A13fs, or A214fs. In one embodiment, the solid tumor (or cancer) has an LTK fusion. In one embodiment, the LTK fusion is CLIP1-LTK. See Cooper A J, Sequist L V, Johnson T W, Lin J J. LTK fusions: A new target emerges in non-small cell lung cancer. Cancer Cell. 2022 Jan. 10; 40 (1): 23-25; and Izumi, H., Matsumoto, S., Liu, J. et al. The CLIP1-LTK fusion is an oncogenic driver in non-small-cell lung cancer. Nature 600, 319-323 (2021), each of which are incorporated herein by reference in their entirety.

In one embodiment, the patient has not been treated with a prior therapy. In one embodiment, the patient is naïve to (i.e. not receiving) any tyrosine kinase inhibitor (TKI) therapy.

In one embodiment, the patient has been treated with one or more prior therapies. In one embodiment, the patient has been treated with at least one prior TKI therapy. In one embodiment, the patient has been treated with at least two prior TKI therapies. In one embodiment, the patient has been treated with one prior TKI therapy. In one embodiment, the patient has been treated with two prior TKI therapies. In one embodiment, the TKI is ROS1 TKI (e.g. crizotinib or entrectinib). In one embodiment, the prior TKI therapy is one or more selected from the group consisting of crizotinib, entrectinib, repotrectinib, taletrectinib, and lorlatinib.

In one embodiment, the patient has not been treated with prior platinum-based chemotherapy. In one embodiment, the patient has been treated with up to one prior platinum-based chemotherapy. In one embodiment, the patient has been treated with at least one prior platinum-based chemotherapy. In one embodiment, the patient has been treated with at least two prior platinum-based chemotherapies. In one embodiment, the patient has been treated with one prior platinum-based chemotherapy. In one embodiment, the patient has been treated with two prior platinum-based chemotherapies.

As used herein, “platinum-based chemotherapy” refers to chemotherapeutic agents that are coordination complexes of platinum. Exemplified platinum-based chemotherapy includes but not limited to cisplatin, oxaliplatin, nedaplatin, or carboplatin.

In one embodiment, the patient has not been treated with immunotherapy. In one embodiment, the patient has been treated with immunotherapy. In one embodiment, the patient has been treated with at least one prior immunotherapy. In one embodiment, the patient has been treated with at least two prior immunotherapies. In one embodiment, the patient has been treated with one prior immunotherapy. In one embodiment, the patient has been treated with two immunotherapies.

As used herein, “immunotherapy” refers to the treatment of a disease by activating or suppressing the immune system. Immunotherapies designed to elicit or amplify an immune response are classified as activation immunotherapies, while immunotherapies that reduce or suppress are classified as suppression immunotherapies. The immunotherapy can regulate the immune effector cells (e.g. lymphocytes, macrophages, dendritic cells, natural killer cells (NK Cell), cytotoxic T lymphocytes (CTL), etc.) to work together against cancer by targeting abnormal antigens expressed on the surface of tumor cells. Exemplified immunotherapy includes but not limited to checkpoint inhibitors (e.g. anti-cytotoxic T-lymphocyte-associated protein 4 (CTLA-4) and anti-programmed cell death protein 1 (PD-1) antibodies). Exemplified PD-1 inhibitors include but are not limited to pembrolizumab (Keytruda), nivolumab (Opdivo), and cemiplimab (Libtayo). Exemplified PD-L1 inhibitors include but are not limited to atezolizumab (Tecentriq), avelumab (Bavencio), durvalumab (Imfinzi). Exemplified CTLA-4 inhibitor includes but is not limited to ipilimumab (Yervoy).

In one embodiment, the patient has not been treated with chemotherapy. In one embodiment, the patient has been treated with at least one prior line of chemotherapy. In one embodiment, the patient has been treated with at least two prior lines of chemotherapy.

In one embodiment, the patient has been treated with at least three prior lines of anticancer therapy. In one embodiment, the patient has been treated with at least two prior lines of anticancer therapy selected from the group consisting of ROS1 TKI (e.g., investigational ROS1 TKI, crizotinib, lorlatinib, entrectinib, taletrectinib, repotrectinib) and chemotherapy.

In one embodiment, the patient has been treated with at least one line of ROS1 TKI and one line of chemotherapy. In one embodiment, the patient has been treated with at least two lines of ROS1 TKI and one line of chemotherapy. In one embodiment, the patient has been treated with at least three lines of ROS1 TKI and one line of chemotherapy. In one embodiment, the patient has been treated with at least two lines of chemotherapy. In one embodiment, the patient has been treated with at least one line of ROS1 TKI and two lines of chemotherapy. In one embodiment, the patient has been treated with at least two lines of ROS1 TKI and two lines of chemotherapy. In one embodiment, the patient has been treated with at least three lines of ROS1 TKI and two lines of chemotherapy.

In one embodiment, the ROS1 TKI is crizotinib. In one embodiment, the ROS1 TKI is entrectinib. In one embodiment, the ROS1 TKI is lorlatinib. In one embodiment, the ROS1 TKI is repotrectinib. In one embodiment, the patient has been treated with lorlatinib and repotrectinib. In one embodiment, the ROS1 TKI is taletrectinib.

In one embodiment, the solid tumor is metastatic ROS1-positive solid tumor, and the patient has been treated with at least one prior ROS1 TKI therapy.

In one embodiment, the solid tumor is metastatic ROS1-positive NSCLC, and the patient is naïve to TKI therapy and has been treated with up to one prior platinum-based chemotherapy with or without immunotherapy.

In one embodiment, the solid tumor is metastatic ROS1-positive NSCLC, and the patient has been treated with one prior ROS1 TKI therapy and has not been treated with prior platinum-based chemotherapy or immunotherapy.

In one embodiment, the solid tumor is metastatic ROS1-positive NSCLC, and the patient has been treated with one prior ROS1 TKI therapy and one prior platinum-based chemotherapy with or without immunotherapy.

In one embodiment, the solid tumor is metastatic ROS1-positive NSCLC, and the patient has been treated with at least two prior ROS1 TKI therapies and up to one prior platinum-based chemotherapy with or without immunotherapy.

In one embodiment, the solid tumor is metastatic ROS1-positive solid tumor, and the patient has progressed on a prior therapy. In one embodiment, the prior therapy is a prior ROS1 TKI therapy. In one embodiment, the prior therapy is a prior chemotherapy (e.g., platinum-based chemotherapy). In one embodiment, the prior therapy is a prior immunotherapy.

In one embodiment, the solid tumor is advanced ROS1-positive NSCLC, and the patient is naïve to TKI therapy and has been treated with up to one prior platinum-based chemotherapy with or without immunotherapy.

In one embodiment, the solid tumor is advanced ROS1-positive NSCLC, and the patient has been treated with one prior ROS1 TKI therapy and has not been treated with prior platinum-based chemotherapy or immunotherapy.

In one embodiment, the solid tumor is advanced ROS1-positive NSCLC, and the patient has been treated with one prior ROS1 TKI therapy and one prior platinum-based chemotherapy with or without immunotherapy.

In one embodiment, the solid tumor is advanced ROS1-positive NSCLC, and the patient has been treated with at least two prior ROS1 TKI therapies and up to one prior platinum-based chemotherapy with or without immunotherapy.

In one embodiment, the solid tumor is advanced ROS1-positive solid tumor, and the patient has progressed on a prior therapy. In one embodiment, the prior therapy is a prior ROS1 TKI therapy.

In one embodiment, the ROS1 TKI is crizotinib. In one embodiment, the ROS1 TKI is entrectinib.

In one embodiment, Compound 1 is administered to the patient for one or more days. In one embodiment, Compound 1 is administered to the patient for at least one treatment cycle. In one embodiment, one treatment cycle is at least 7 days. In one embodiment, one treatment cycle is at least 14 days. In one embodiment, one treatment cycle is at least 21 days. In one embodiment, one treatment cycle is at least 28 days.

In one embodiment, the patient does not experience a Grade 4 adverse event after the administration of Compound 1. In one embodiment, the patient does not experience a Grade 3 adverse event after the administration of Compound 1. In one embodiment, the patient does not experience a Grade 2 adverse event after the administration of Compound 1. In one embodiment, the patient does not experience a Grade 1 adverse event after the administration of Compound 1. As used here and unless otherwise specified, the grade of the adverse event follows the National Cancer Institute Common Terminology Criteria for Adverse Events (CTCAE) grades.

In one embodiment, the patient only experiences nausea after the administration of Compound 1. In one embodiment, the patient does not experience a neurologic adverse event after the administration of Compound 1. In one embodiment, the patient does not experience one or more CNS adverse event selected from the group consisting of dizziness, ataxia, gait disturbance, paraesthesia, weight gain, hyperphagia, paresthesias, abnormal movement, cognitive changes, seizures, hallucinations, speech effects (e.g., aphasia, dysarthria, slow speech, or speech disorder), mood disorder (e.g., irritability, anxiety, depression, affective disorder, affect lability, personality change, mood swings, affective disorder, aggression, stress, agitation, mood altered, depressed mood, euphoric mood, suicidal ideation, or mania), mental status, sleep disorder, and cognitive disorder (e.g., memory impairment, cognitive disorder, amnesia, confusion, disturbance in attention, delirium, mental impairment, attention deficit/hyperactivity disorder, dementia, sleep disturbance, disorientation, or reading disorder). In one embodiment, the neurologic adverse event is one or more selected from the group consisting of cognitive impairment, mood disorders, sleep disturbances, dizziness, and ataxia. In one embodiment, the patient does not experience an adverse event of weight gain and/or glucose metabolism disorders. In one embodiment, the glucose metabolism disorder is hyperglycemia (e.g., diabetes). In one embodiment, the glucose metabolism disorder is hypoglycemia. In one embodiment, the patient experiences a Grade 1 treatment-related adverse event after the administration of Compound 1. In one embodiment, the patient experiences at most a Grade 1 treatment-related adverse event after the administration of Compound 1. In one embodiment, the Grade 1 treatment-related adverse event is fatigue, myalgia, oedema (e.g., oedema and oedema peripheral), increased Alanine aminotransferase (ALT), increased Aspartate aminotransferase (AST), or nausea.

In one embodiment, the patient experiences no treatment-related serious adverse event (SAE). In one embodiment, the patient experiences no treatment-related CNS adverse event. In one embodiment, the patient experiences no treatment-related dizziness.

In one embodiment, less than about 40% of the patient population experiences at most a Grade 1 adverse event (e.g., TRAE) after the administration of Compound 1. In one embodiment, less than about 30% of the patient population experiences at most a Grade 1 adverse event (e.g., TRAE) after the administration of Compound 1. In one embodiment, less than about 20% of the patient population experiences at most a Grade 1 adverse event (e.g., TRAE) after the administration of Compound 1. In one embodiment, less than about 15% of the patient population experiences at most a Grade 1 adverse event (e.g., TRAE) after the administration of Compound 1. In one embodiment, the Grade 1 adverse event (e.g., TRAE) is fatigue, myalgia, oedema (e.g., oedema and oedema peripheral), increased alanine aminotransferase (ALT), increased aspartate aminotransferase (AST), or nausea.

In one embodiment, less than about 30% of the patient population experiences a Grade 2 adverse event (e.g., TRAE) after the administration of Compound 1. In one embodiment, less than about 20% of the patient population experiences a Grade 2 adverse event (e.g., TRAE) after the administration of Compound 1. In one embodiment, less than about 15% of the patient population experiences a Grade 2 adverse event (e.g., TRAE) after the administration of Compound 1. In one embodiment, less than about 10% of the patient population experiences a Grade 2 adverse event (e.g., TRAE) after the administration of Compound 1. In one embodiment, less than about 5% of the patient population experiences a Grade 2 adverse event (e.g., TRAE) after the administration of Compound 1. In one embodiment, the patient population experiences no Grade 2 adverse event (e.g., TRAE) after the administration of Compound 1.

In one embodiment, less than about 20% of the patient population experiences a Grade 3 adverse event (e.g., TRAE) after the administration of Compound 1. In one embodiment, less than about 15% of the patient population experiences a Grade 3 adverse event (e.g., TRAE) after the administration of Compound 1. In one embodiment, less than about 10% of the patient population experiences a Grade 3 adverse event (e.g., TRAE) after the administration of Compound 1. In one embodiment, less than about 5% of the patient population experiences a Grade 3 adverse event (e.g., TRAE) after the administration of Compound 1. In one embodiment, the patient population experiences no Grade 3 adverse event (e.g., TRAE) after the administration of Compound 1.

In one embodiment, less than about 10% of the patient population experiences a Grade 4 adverse event (e.g., TRAE) after the administration of Compound 1. In one embodiment, less than about 5% of the patient population experiences a Grade 4 adverse event (e.g., TRAE) after the administration of Compound 1. In one embodiment, less than about 1% of the patient population experiences a Grade 4 adverse event (e.g., TRAE) after the administration of Compound 1. In one embodiment, the patient population experiences no Grade 4 adverse event (e.g., TRAE) after the administration of Compound 1.

In one embodiment, the patient has a complete response after one or more cycles of treatment. In one embodiment, the patient has a partial response after one or more cycles of treatment. In one embodiment, the patient has reached stable disease after one or more cycles of treatment. In one embodiment, the patient has not experienced progressive disease after one or more cycles of treatment.

As used herein and unless otherwise specified, Complete Response (CR) refers to the disappearance of all target lesion(s). In some embodiments of CR, any pathological lymph nodes (whether target or non-target) must have reduction in short axis to <10 mm. Partial Response (PR) refers to at least a 30% decrease in the sum of diameters of target lesion(s). Progressive Disease (PD) refers to at least a 20% increase in the sum of diameters of target lesion(s). In some embodiments of PD, in addition to the relative increase of 20%, the sum also demonstrates an increase of at least 5 mm. In some embodiments, the appearance of one or more new lesion(s) is also considered progression. Stable Disease (SD) refers to neither sufficient shrinkage to qualify for PR nor sufficient increase to qualify for PD, taking as reference the smallest sum diameters while on study. Additional description of these terms according to RECIST 1.1 can be found in EUROPEAN JOURNAL OF CANCER 45 (2009) 228-247.

In one embodiment, the patient has brain metastases. In one embodiment, the patient has brain metastases and experiences no intracranial progression after at least one treatment cycle.

In one embodiment, the patient has at least about 5% to about 100% reduction of ROS1 allele variant in circulating tumor DNA after at least one treatment cycle. In one embodiment, the patient has at least about 35% reduction of ROS1 allele variant in circulating tumor DNA after at least one treatment cycle. In one embodiment, the patient has at least about 45% reduction of ROS1 allele variant in circulating tumor DNA after at least one treatment cycle. In one embodiment, the patient has at least about 55% reduction of ROS1 allele variant in circulating tumor DNA after at least one treatment cycle. In one embodiment, the patient has at least about 65% reduction of ROS1 allele variant in circulating tumor DNA after at least one treatment cycle. In one embodiment, the patient has at least about 75% reduction of ROS1 allele variant in circulating tumor DNA after at least one treatment cycle. In one embodiment, the patient has at least about 85% reduction of ROS1 allele variant in circulating tumor DNA after at least one treatment cycle. In one embodiment, the patient has at least about 100% reduction of ROS1 allele variant in circulating tumor DNA after at least one treatment cycle. In one embodiment, the patient has undetectable ROS1 allele variant in circulating tumor DNA after at least one treatment cycle. In one embodiment, the ROS1 allele variant is G2032R.

In one embodiment, the prior therapy is a prior ROS1 TKI therapy. In one embodiment, the ROS1 TKI is crizotinib, entrectinib, lorlatinib, ceritinib, cabozantinib, taletrectinib, or repotrectinib.

In one embodiment, administration of Compound 1 provides the area under the curve from 0 to 24 hours (AUC₀₋₂₄) of the compound in a range of (80% to 125% of 500 ng*h/mL) to (80% to 125% of 30000 ng*h/mL). In one embodiment, administration of Compound 1 provides the area under the curve from 0 to 24 hours (AUC₀₋₂₄) of the compound in a range of about 500 to about 30000 ng*h/mL. In one embodiment, the area under the curve from 0 to 24 hours (AUC₀₋₂₄) of the compound is in a range of about 1000 to about 13000 ng*h/mL after about half cycle of treatment (e.g., 15 days). In one embodiment, the area under the curve from 0 to 24 hours (AUC₀₋₂₄) of the compound is in a range of about 1500 to about 10000 ng*h/mL after about half cycle of treatment (e.g., 15 days). In one embodiment, the area under the curve from 0 to 24 hours (AUC₀₋₂₄) of the compound is in a range of about 2000 to about 8000 ng*h/mL after about half cycle of treatment (e.g., 15 days).

In one embodiment, such administration provides the area under the curve from 0 to 24 hours after administration (AUC₀₋₂₄) of the compound in a range of (80% to 125% of 20 ng*h/mL) to (80% to 125% of 500 ng*h/mL) for every mg of Compound 1 administered. In one embodiment, such administration provides the area under the curve from 0 to 24 hours after administration (AUC₀₋₂₄) of the compound in a range of from about 20 to about 500 ng*h/mL for every mg of Compound 1 administered. In one embodiment, such administration provides the area under the curve from 0 to 24 hours after administration (AUC₀₋₂₄) of the compound in a range of from about 50 to about 200 ng*h/mL after about half cycle of treatment (e.g., 15 days).

In one embodiment, administration of Compound 1 provides the area under the curve of unbound compound 1 from 0 hours to the last measurable plasma concentration (AUC_{last,unbound} of the compound in a range of (80% to 125% of 400 ng*h/mL) to (80% to 125% of 7000 ng*h/mL). In one embodiment, administration of Compound 1 provides AUC_{last,unbound} of the compound in a range of about 400 to about 7000 ng*h/mL. In one embodiment, the AUC_{last,unbound} of the compound is in a range of about 1000 to about 6200 ng*h/mL. In one embodiment, the AUC_{last,unbound} of the compound is in a range of about 1100 to about 1500 ng*h/mL following administration of about 25 mg of the compound. In one embodiment, the AUC_{last,unbound} of the compound is in a range of about 2800 to about 3200 ng*h/mL following administration of about 50 mg of the compound. In one embodiment, the AUC_{last,unbound} of the compound is in a range of about 3800 to about 4200 ng*h/mL following administration of about 75 mg of the compound. In one embodiment, the AUC_{last,unbound} of the compound is in a range of about 5500 to about 5900 ng*h/mL following administration of about 100 mg of the compound. In one embodiment, the AUC_{last,unbound} of the compound is in a range of about 4800 to about 5200 ng*h/mL following administration of about 125 mg of the compound. In one embodiment, the AUC_{last,unbound} of the compound is in a range of about 2600 to about 3100 ng*h/mL following administration of about 150 mg of the compound. In one embodiment, the AUC_{last,unbound} of the compound is about 2850 ng*h/mL following administration of about 150 mg of the compound. In certain embodiments, the last measurable plasma concentration is at 24 hours.

In one embodiment, such administration provides the area under the curve of unbound compound from 0 hours to the last measurable plasma concentration (AUC_{last,unbound}) of the compound in a range of (80% to 125% of 10 ng*h/mL) to (80% to 125% of 80 ng*h/mL) for every mg of Compound 1 administered. In one embodiment, such administration provides the AUC_{last,unbound} of the compound in a range of from about 10 to about 80 ng*h/mL for every mg of Compound 1 administered. In one embodiment, such administration provides the AUC_{last,unbound} of the compound in a range of from about 30 to about 70 ng*h/mL for every mg of Compound 1 administered. In one embodiment, such administration provides the AUC_{last,unbound} of the compound in a range of from about 35 to about 65 ng*h/mL for every mg of Compound 1 administered. In one embodiment, the AUC_{last,unbound} of the compound is in a range of from about 15 to about 25 ng*h/mL for every mg of Compound 1 administered. In one embodiment, the AUC_{last,unbound} of the compound is about 19 ng*h/mL for every mg of Compound 1 administered. In certain embodiments, the last measurable plasma concentration is at 24 hours.

In one embodiment, administration of Compound 1 provides the area under the curve from 0 to the end of the dosing interval (AUCₜₐᵤ) of the compound in a range of 2000 hr*ng/mL to 10000 hr*ng/mL. In one embodiment, administration of Compound 1 provides the AUCₜₐᵤ of the compound in a range of about 2000 to about 8000 ng*h/mL. In one embodiment, administration of Compound 1 provides the AUCₜₐᵤ of the compound in a range of about 2000 to about 8000 ng*h/mL after about half cycle of treatment (e.g., 15 days).

In one embodiment, administration of Compound 1 provides the area under the curve from 0 to the end of the dosing interval (AUCₜₐᵤ) of the compound in a range of 30 hr*ng/ml to 200 hr*ng/mL for every mg of Compound 1 administered. In one embodiment, administration of Compound 1 provides the AUCₜₐᵤ of the compound in a range of about 50 to about 150 ng*h/mL for every mg of Compound 1 administered. In one embodiment, administration of Compound 1 provides the AUCₜₐᵤ of the compound in a range of about 50 to about 150 ng*h/mL for every mg of Compound 1 administered after about half cycle of treatment (e.g., 15 days).

In one embodiment, administration of Compound 1 provides the area under the curve from 0 to infinity (AUC_{inf}) of the compound in a range of 3500 hr*ng/ml to 20000 hr*ng/mL. In one embodiment, administration of Compound 1 provides the AUC_{inf} of the compound in a range of about 4000 to about 15000 ng*h/mL. In one embodiment, administration of Compound 1 provides the AUC_{inf} of the compound in a range of about 4000 to about 15000 ng*h/mL after about half cycle of treatment (e.g., 15 days).

In one embodiment, administration of Compound 1 provides the area under the curve from 0 to infinity (AUC_{inf}) of the compound in a range of 50 hr*ng/ml to 300 hr*ng/ml for every mg of Compound 1 administered. In one embodiment, administration of Compound 1 provides the AUC_{inf} of the compound in a range of about 50 to about 250 ng*h/mL for every mg of Compound 1 administered. In one embodiment, administration of Compound 1 provides the AUC_{inf} of the compound in a range of about 50 to about 250 ng*h/mL for every mg of Compound 1 administered after about half cycle of treatment (e.g., 15 days).

In one embodiment, such administration provides the maximum plasma concentration (Cₘₐₓ) of the compound in a range of (80% to 125% of 100 ng/ml) to (80% to 125% of 1500 ng/mL). In one embodiment, such administration provides the maximum plasma concentration (Cₘₐₓ) of the compound in a range of about 100 to about 1500 ng/ml. In one embodiment, such administration provides the maximum plasma concentration (Cₘₐₓ) of the compound in a range of about 200 to about 1000 ng/mL. In one embodiment, administration of Compound 1 provides the Cₘₐₓ of the compound in a range of about 200 to about 1000 ng*h/mL after about half cycle of treatment (e.g., 15 days).

In one embodiment, such administration provides the maximum plasma concentration (Cₘₐₓ) of the compound in a range of about 2 to about 50 ng/mL for every mg of Compound 1 administered. In one embodiment, such administration provides the maximum plasma concentration (Cₘₐₓ) of the compound in a range of about 5 to about 20 ng/ml for every mg of Compound 1 administered. In one embodiment, administration of Compound 1 provides the Cₘₐₓ of the compound in a range of about 5 to about 20 ng/mL for every mg of Compound 1 administered after about half cycle of treatment (e.g., 15 days).

In one embodiment, such administration provides the minimum plasma concentration reached by Compound 1 during the time interval between administration of two doses (Cₘᵢₙ) in a range of about 50 to about 400 ng/ml. In one embodiment, such administration provides the minimum plasma concentration reached by Compound 1 during the time interval between administration of two doses (Cₘᵢₙ) in a range of about 50 to about 350 ng/mL. In one embodiment, such administration provides the minimum plasma concentration reached by Compound 1 during the time interval between administration of two doses (Cₘᵢₙ) in a range of about 50 to about 350 ng/mL after about half cycle of treatment (e.g., 15 days).

In one embodiment, such administration provides the maximum plasma concentration of unbound compound (C_{max,unbound}) in a range of (80% to 125% of 30 ng/ml) to (80% to 125% of 400 ng/mL). In one embodiment, such administration provides the unbound maximum plasma concentration (C_{max,unbound}) of the compound in a range of about 30 ng/ml to about 400 ng/mL. In one embodiment, such administration provides the unbound maximum plasma concentration (C_{max,unbound}) of the compound in a range of about 50 ng/ml to about 350 ng/mL. In one embodiment, the C_{max,unbound} of the compound is in a range of about 90 to about 140 ng/mL following administration of about 50 mg of the compound. In one embodiment, the C_{max,unbound} of the compound is in a range of about 200 to about 250 ng/ml following administration of about 75 mg of the compound. In one embodiment, the C_{max,unbound} of the compound is in a range of about 300 to about 400 ng/mL following administration of about 100 mg of the compound. In one embodiment, the C_{max,unbound} of the compound is in a range of about 200 to about 300 ng/ml following administration of about 150 mg of the compound. In one embodiment, the C_{max,unbound} of the compound is in a range of about 200 to about 300 ng/ml following administration of about 150 mg of the compound. In one embodiment, the C_{max,unbound} of the compound is about 258 ng/mL (about 0.62 μM) following administration of about 150 mg of the compound. In one embodiment, the C_{max,unbound} of the compound is in a range of about 150 to about 200 ng/ml following administration of about 100 mg of the compound. In one embodiment, the C_{max,unbound} of the compound is about 172 ng/ml (about 0.41 μM) following administration of about 100 mg of the compound. In one embodiment, such administration provides the C_{max,unbound} of the compound in a range of about 1 to about 4 ng/ml for every mg of Compound 1 administered. In one embodiment, the C_{max,unbound} of the compound is about 1.7 ng/ml for every mg of Compound 1 administered.

In one embodiment, without being bound by a particular theory, the brain penetration of Compound 1 (brain to plasma ratio) is about 0.26 (about 26%).

In one embodiment, such administration provides the Tₘₐₓ of the compound after the administration in a range of about 0.25h to about 5h. In one embodiment, such administration provides the Tₘₐₓ of the compound after the administration in a range of about 0.25h to about 4h. In one embodiment, such administration provides the Tₘₐₓ of the compound after the administration in a range of about 0.5h to about 5h. In one embodiment, such administration provides the Tₘₐₓ of the compound after the administration in a range of about 0.5h to about 2h. In one embodiment, such administration provides the Tₘₐₓ of the compound after the administration in a range of about 0.5h to about 1.0h. In one embodiment, such administration provides the Tₘₐₓ of the compound after the administration of about 1h. In one embodiment, such administration provides the Tₘₐₓ of the compound after the administration of about 0.5h.

In one embodiment, such administration provides the t_{1/2} of the compound after the administration in a range of about 2h to about 50h. In one embodiment, such administration provides the t_{1/2} of the compound after the administration in a range of about 8h to about 25h. In one embodiment, such administration provides the t_{1/2} of the compound after the administration in a range of about 10h to about 24h. In one embodiment, such administration provides the t_{1/2} of the compound after the administration in a range of about 10h to about 20h. In one embodiment, such administration provides the t_{1/2} of the compound after the administration in a range of about 16h to about 25h. In one embodiment, such administration provides the t_{1/2} of the compound after the administration of about 10h, about 11h, about 12h, about 13h, about 14h, about 15h, about 16h, about 17h, about 18h, about 19h, about 20h, about 21h, about 22h, about 23h, or about 24h. In one embodiment, such administration provides the tin of the compound after the administration of about 20h.

In one embodiment, the administration provides the dose plasma concentration of the compound at least 10%, 20%, 30%, 40%, or 50% higher than the IC₅₀ of the compound against ROS1 G2032R mutant during at least 70%, 80%, 90%, 95%, 97%, 98%, or 99% of about 24 hours immediately following the administration.

A method of reducing a lesion in a subject having a ROS1-positive solid tumor (e.g., NSCLC), comprising
(i) obtaining a first radiological measurement of the size of the lesion;
(ii) administering a pharmaceutically effective amount of Compound 1 once daily for one or more days; and
(iii) obtaining a second radiological measurement of the size of the lesion; wherein the second measurement is at most 100% of the first measurement.

In one embodiment, the second measurement is at most about 90% of the first measurement. In one embodiment, the second measurement is at most about 80% of the first measurement. In one embodiment, the second measurement is at most about 70% of the first measurement. In one embodiment, the second measurement is at most about 60% of the first measurement. In one embodiment, the second measurement is at most about 50% of the first measurement. In one embodiment, the second measurement is about 0.01% to about 90% of the first measurement. In one embodiment, the second measurement shows no detectable lesion.

In one embodiment, the compound used herein (Compound 1 or a stereoisomer, or a mixture of stereoisomers thereof, or a pharmaceutically acceptable salt thereof) is administered once daily (QD). In one embodiment, the compound is administered twice daily (BID). In certain embodiments, the compound used herein is Compound 1.

In one embodiment, the compound is administered at an amount such that the area under the curve from 0 to 24 hours after administration (AUC₀₋₂₄) of the compound is in a range of from about 500 to about 30000 ng*h/mL. In one embodiment, the AUC₀₋₂₄ is in a range of from about 750 to about 20000 ng*h/mL. In one embodiment, the AUC₀₋₂₄ is in a range of from about 750 to about 15000 ng*h/mL. In one embodiment, the AUC₀₋₂₄ is in a range of from about 1000 to about 13000 ng*h/mL. In one embodiment, the AUC₀₋₂₄ is in a range of from about 1500 to about 10000 ng*h/mL. In one embodiment, the AUC₀₋₂₄ is in a range of from about 750 to about 4500 ng*h/mL. In one embodiment, the AUC₀₋₂₄ is in a range of from about 1000 to about 3000 ng*h/mL. In one embodiment, the AUC₀₋₂₄ is in a range of from about 1250 to about 3000 ng*h/mL. In one embodiment, the AUC₀₋₂₄ is in a range of from about 1500 to about 2500 ng*h/mL. In one embodiment, the AUC₀₋₂₄ is in a range of from about 1750 to about 7500 ng*h/mL. In one embodiment, the AUC₀₋₂₄ is in a range of from about 2000 to about 6000 ng*h/mL. In one embodiment, the AUC₀₋₂₄ is in a range of from about 3000 to about 5000 ng*h/mL. In one embodiment, the AUC₀₋₂₄ is in a range of from about 3000 to about 14000 ng*h/mL. In one embodiment, the AUC₀₋₂₄ is in a range of from about 3000 to about 9000 ng*h/mL. In one embodiment, the AUC₀₋₂₄ is in a range of from about 4500 to about 7500 ng*h/mL. In one embodiment, the AUC₀₋₂₄ is in a range of from about 4000 to about 13000 ng*h/mL. In one embodiment, the AUC₀₋₂₄ is in a range of from about 6000 to about 10000 ng*h/mL.

In one embodiment, the compound is administered at an amount such that the maximum plasma concentration (Cₘₐₓ) of the compound is in a range of from about 100 to about 1500 ng/mL. In one embodiment, the Cₘₐₓ is in a range of from about 150 to about 1400 ng/mL. In one embodiment, the Cₘₐₓ is in a range of from about 150 to about 350 ng/ml. In one embodiment, the Cₘₐₓ is in a range of from about 300 to about 900 ng/mL. In one embodiment, the Cₘₐₓ is in a range of from about 300 to about 700 ng/mL. In one embodiment, the Cₘₐₓ is in a range of from about 450 to about 1050 ng/mL. In one embodiment, the Cₘₐₓ is in a range of from about 600 to about 1400 ng/mL.

In one embodiment, the compound is administered at an amount such that the plasma concentration of the compound is higher (e.g., at least 10%, 20%, 30%, 40%, or 50% higher) than a predetermined value (e.g., the IC₅₀ of the compound against ROS1 G2032R mutant) during at least 70%, 80%, 90%, 95%, 97%, 98%, or 99% of a selected time period (e.g., about 24 hours) immediately following the administration.

In one embodiment, the compound has a half-life of about 2 to about 50 hours in the patient. In one embodiment, the half-life of about 10 to about 20 hours. In one embodiment, the half-life of about 15 to about 25 hours. In one embodiment, the half-life is about 20 hours.

In one embodiment, the pharmacokinetic parameters provided herein (e.g., AUC₀-24, AUC_{last,unbound}, Cₘₐₓ, Tₘₐₓ, and half-life) refers to an arithmetic mean of a patient population. In one embodiment, the pharmacokinetic parameters provided herein (e.g., AUC₀₋₂₄, Cₘₐₓ) refers to a geometric mean of a patient population. In certain embodiments, t_{1/2} refers to the arithmetic mean of a patient population. In certain embodiments, Tₘₐₓ refers to the median range of a patient population. In certain embodiments, each of AUC₀₋₂₄, AUC_{last,unbound}, AUCₜₐᵤ, AUC_{tau_DN}, AUC_{inf}, AUC_{inf_DN}, Cₘₐₓ, C_{max_DN}, Cₘₐₓ unbound, and Cₘᵢₙ refers to the geometric mean of a patient population.

In one embodiment, the pharmacokinetic parameters provided herein (e.g., AUC₀₋₂₄, Cₘₐₓ, Tₘₐₓ, and half-life) are measured after the compound is administered to the patient for the first time in a treatment cycle (e.g., day 1 of a treatment cycle, e.g., day 1 of cycle 1 of a 28-day treatment cycle). In one embodiment, the pharmacokinetic parameters provided herein (e.g., AUC₀₋₂₄, Cₘₐₓ, and half-life) are measured after the compound has been administered to the patient multiple times in a treatment cycle (e.g., a steady state in the patient may have been achieved for the compound) (e.g., day 15 of cycle 1 of a 28-day treatment cycle).

In one embodiment, the compound used herein (Compound 1 or a stereoisomer, or a mixture of stereoisomers thereof, or a pharmaceutically acceptable salt thereof) is administered at an amount of from about 5 mg to about 500 mg (by weight of the free base Compound 1) per day. In one embodiment, the compound is administered at an amount of from about 25 mg to about 250 mg per day. In one embodiment, the compound is administered at an amount of from about 25 mg to about 200 mg per day. In one embodiment, the compound is administered at an amount of from about 50 mg to about 200 mg per day. In one embodiment, the compound is administered at an amount of from about 5 mg to about 150 mg per day. In one embodiment, the compound is administered at an amount of from about 25 mg to about 150 mg per day. In one embodiment, the compound is administered at an amount of from about 25 mg to about 125 mg per day. In one embodiment, the compound is administered at an amount of from about 25 mg to about 100 mg per day. In one embodiment, the compound is administered at an amount of from about 50 mg to about 125 mg per day. In one embodiment, the compound is administered at an amount of from about 50 mg to about 100 mg per day. In one embodiment, the compound is administered at an amount of about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, or about 200 mg per day. In one embodiment, the amount is about 5 mg per day. In one embodiment, the amount is about 10 mg per day. In one embodiment, the amount is about 15 mg per day. In one embodiment, the amount is about 20 mg per day. In one embodiment, the amount is about 25 mg per day. In one embodiment, the amount is about 30 mg per day. In one embodiment, the amount is about 35 mg per day. In one embodiment, the amount is about 40 mg per day. In one embodiment, the amount is about 45 mg per day. In one embodiment, the amount is about 50 mg per day. In one embodiment, the amount is about 75 mg per day. In one embodiment, the amount is about 100 mg per day. In one embodiment, the amount is about 125 mg per day. In one embodiment, the amount is about 150 mg per day. As used herein, the weight amount refers to the weight amount of the free base Compound 1. In certain embodiments, the compound used herein is Compound 1.

In one embodiment, the compound used herein (Compound 1 or a stereoisomer, or a mixture of stereoisomers thereof, or a pharmaceutically acceptable salt thereof) is administered at an amount of from about 5 mg to about 500 mg (by weight of the free base, Compound 1) once daily. In one embodiment, the compound is administered at an amount of from about 25 mg to about 250 mg once daily. In one embodiment, the compound is administered at an amount of from about 25 mg to about 200 mg once daily. In one embodiment, the compound is administered at an amount of from about 50 mg to about 200 mg once daily. In one embodiment, the compound is administered at an amount of from about 5 mg to about 150 mg once daily. In one embodiment, the compound is administered at an amount of from about 25 mg to about 150 mg once daily. In one embodiment, the compound is administered at an amount of from about 25 mg to about 125 mg once daily. In one embodiment, the compound is administered at an amount of from about 25 mg to about 100 mg once daily. In one embodiment, the compound is administered at an amount of from about 50 mg to about 125 mg once daily. In one embodiment, the compound is administered at an amount of from about 50 mg to about 100 mg once daily. In one embodiment, the compound is administered at an amount of about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, or about 200 mg once daily. In one embodiment, the amount is about 5 mg once daily. In one embodiment, the amount is about 10 mg once daily. In one embodiment, the amount is about 15 mg once daily. In one embodiment, the amount is about 20 mg once daily. In one embodiment, the amount is about 25 mg once daily. In one embodiment, the amount is about 30 mg once daily. In one embodiment, the amount is about 35 mg once daily. In one embodiment, the amount is about 40 mg once daily. In one embodiment, the amount is about 45 mg once daily. In one embodiment, the amount is about 50 mg once daily. In one embodiment, the amount is about 75 mg once daily. In one embodiment, the amount is about 100 mg once daily. In one embodiment, the amount is about 125 mg once daily. In one embodiment, the amount is about 150 mg once daily. As used herein, the weight amount refers to the weight amount of the free base Compound 1. In certain embodiments, the compound used herein is Compound 1.

In one embodiment, the compound used herein (Compound 1 or a stereoisomer, or a mixture of stereoisomers thereof, or a pharmaceutically acceptable salt thereof) is administered at an amount of from about 5 mg to about 500 mg (by weight of the free base, Compound 1) twice daily. In one embodiment, the compound is administered at an amount of from about 5 mg to about 250 mg twice daily. In one embodiment, the compound is administered at an amount of from about 25 mg to about 250 mg twice daily. In one embodiment, the compound is administered at an amount of from about 25 mg to about 200 mg twice daily. In one embodiment, the compound is administered at an amount of from about 5 mg to about 100 mg twice daily. In one embodiment, the compound is administered at an amount of from about 10 mg to about 50 mg twice daily. In one embodiment, the compound is administered at an amount of from about 10 mg to about 100 mg twice daily. In one embodiment, the compound is administered at an amount of from about 25 mg to about 100 mg twice daily. In one embodiment, the compound is administered at an amount of from about 50 mg to about 100 mg twice daily. In one embodiment, the compound is administered at an amount of from about 5 mg to about 150 mg twice daily. In one embodiment, the compound is administered at an amount of from about 25 mg to about 150 mg twice daily. In one embodiment, the compound is administered at an amount of about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, or about 200 mg twice daily. In one embodiment, the amount is about 5 mg twice daily. In one embodiment, the amount is about 10 mg twice daily. In one embodiment, the amount is about 15 mg twice daily. In one embodiment, the amount is about 20 mg twice daily. In one embodiment, the amount is about 25 mg twice daily. In one embodiment, the amount is about 30 mg twice daily. In one embodiment, the amount is about 35 mg twice daily. In one embodiment, the amount is about 40 mg twice daily. In one embodiment, the amount is about 45 mg twice daily. In one embodiment, the amount is about 50 mg twice daily. In one embodiment, the amount is about 75 mg twice daily. In one embodiment, the amount is about 100 mg twice daily. In one embodiment, the amount is about 125 mg twice daily. In one embodiment, the amount is about 150 mg twice daily. As used herein, the weight amount refers to the weight amount of the free base Compound 1. In certain embodiments, the compound used herein is Compound 1.

In one embodiment, the compound is administered orally.

In one embodiment, the compound is administered in the form of one or more tablets. In one embodiment, the tablet has a unit dose strength of about 5 mg by weight of the free base Compound 1. In one embodiment, the table has a unit dose strength of about 25 mg by weight of the free base Compound 1.

In one embodiment, the compound is administered to a subject with an empty stomach. In one embodiment, the compound is administered to a subject at fasted status. In one embodiment, the compound is administered to a subject without food. In one embodiment, the compound is administered to a subject at least 1 hour before and no sooner than 2 hours after ingestion of food and/or beverages other than water. In one embodiment, the compound is administered to a subject with a full stomach. In one embodiment, the compound is administered to a subject at fed status. In one embodiment, the compound is administered to a subject with food. In one embodiment, the compound is administered with the ingestion of food and/or beverages.

In one embodiment, the patient experiences improvement in one or more symptoms selected from the group consisting of cognitive impairment, mood disorders, sleep disturbances, dizziness, ataxia, and weight gain, after the administration of the compound. In one embodiment, the patient does not experience one or more symptoms selected from the group consisting of cognitive impairment, mood disorders, sleep disturbances, dizziness, ataxia, and weight gain, after the administration of the compound. In one embodiment, the patient experiences reduced levels of one or more of pROS1, ROS1, pAKT, and pERK, after the administration of the compound. In one embodiment, the patient experiences reduced expression level of one or more MAP kinase pathway genes in tumor, after the administration of the compound. In one embodiment, the patient experiences reduced expression level of one or more MAP kinase pathway genes in solid tumor, after the administration of the compound. In one embodiment, the one or more MAP kinase pathway genes are selected from the group consisting of DUSP6, FOS, ILIR1, and SPRY4.

Cancer is a disease of uncontrolled cell proliferation that results from alterations in certain genes. Some of these alterations occur in genes that encode receptor tyrosine kinases (RTKs), a family of membrane-bound proteins that transmit signals from outside the cell to promote cell survival, growth, and proliferation. Aberrant RTK activation can lead to excessive cell growth and hence cancer. Generally, RTKs contain an N-terminal domain that binds extracellular ligands, a transmembrane domain, and a C-terminal kinase domain that catalyzes intracellular signal transduction.

In some embodiments, the compound of Formula (I) is an inhibitor of human ROS1. ROS1 is an RTK encoded by the ROS1 gene. The ligands and biological functions of human ROS1 are unknown, but its homologs in some other species have been shown to bind extracellular ligands and stimulate cell differentiation. For example, mouse ROS1 is essential for male gamete maturation and reproduction. In humans, ROS1 chromosomal rearrangements are a well-documented cause of cancer, representing 1-2% of non-small cell lung cancer (NSCLC) and a subset of many other cancers. These rearrangements result in the fusion of the C-terminus of ROS1 with the N-terminus of various partner proteins, the most common of which is CD74. ROS1 fusions have constitutive kinase activity that drives tumor growth through MAPK, PI3K, and JAK/STAT signaling pathways. Small-molecule tyrosine kinase inhibitors (TKIs) have been used to target ROS1 fusions in cancer, including crizotinib and entrectinib. Crizotinib was the first FDA-approved TKI for the treatment of ROS1-positive NSCLC. Despite an initial response, most patients acquire resistance to crizotinib and relapse. The predominant mechanism of resistance is the G2032R mutation in the solvent front, which dramatically reduces crizotinib affinity. No inhibitors with activity against ROS1-G2032R fusions have been FDA-approved, indicating a need in the art.

In one embodiment, a compound provided herein selectively inhibits ROS1. In one embodiment, the compound selectively inhibits ROS1 over ALK. By way of non-limiting example, the ratio of selectivity can be greater than a factor of about 1.5, greater than a factor of about 2, greater than a factor of about 3, greater than a factor of about 4, greater than a factor of about 5, greater than a factor of about 10, greater than a factor of about 20, greater than a factor of about 30, greater than a factor of about 50, or greater than a factor of about 100, where selectivity can be measured by ratio of IC₅₀ values, among other means. In one embodiment, the selectivity of ROS1 over ALK is measured by the ratio of the IC₅₀ value against ALK to the IC₅₀ value against ROS1.

In one embodiment, the compound selectively inhibits ROS1 over TRK (e.g., TRKA, TRKB, and/or TRBC). By way of non-limiting example, the ratio of selectivity can be greater than a factor of about 5, greater than a factor of about 10, greater than a factor of about 50, greater than a factor of about 100, greater than a factor of about 200, greater than a factor of about 400, greater than a factor of about 600, greater than a factor of about 800, greater than a factor of about 1000, greater than a factor of about 1500, greater than a factor of about 2000, greater than a factor of about 5000, greater than a factor of about 10,000, or greater than a factor of about 20,000, where selectivity can be measured by ratio of IC₅₀ values, among other means. In one embodiment, the selectivity of ROS1 over TRK is measured by the ratio of the IC₅₀ value against TRK to the IC₅₀ value against ROS1.

In one embodiment, provided herein is a method for selectively inhibiting ROS1 over ALK wherein the inhibition takes place in a cell. In one embodiment, provided herein is a method for selectively inhibiting ROS1 over TRK (e.g., TRKA, TRKB, and/or TRBC) wherein the inhibition takes place in a cell. In one embodiment, the method comprises contacting ROS1 with an effective amount of a compound provided herein. In an embodiment, such contact occurs in a cell. In an embodiment, such contact occurs in a cell in a mammal such as a human. In an embodiment, such contact occurs in a cell in human patient having a cancer provided herein.

In one embodiment, provided herein is a method for selectively inhibiting ROS1 over ALK wherein the inhibition takes place in a subject suffering from cancer, said method comprising administering an effective amount of a compound or a pharmaceutical composition provided herein to said subject. In certain embodiments, provided herein is a method of treating a subject suffering from a cancer associated with ROS1, said method comprising selectively inhibiting ROS1 over ALK by administering an amount of a compound or a pharmaceutical composition provided herein to said subject, wherein said amount is sufficient for selective inhibiting ROS1 over ALK.

In one embodiment, provided herein is a method for selectively inhibiting ROS1 over TRK (e.g., TRKA, TRKB, and/or TRBC) wherein the inhibition takes place in a subject suffering from cancer, said method comprising administering an effective amount of a compound or a pharmaceutical composition provided herein to said subject. In certain embodiments, provided herein is a method of treating a subject suffering from a cancer associated with ROS1, said method comprising selectively inhibiting ROS1 over TRK (e.g., TRKA, TRKB, and/or TRBC) by administering an amount of a compound or a pharmaceutical composition provided herein to said subject, wherein said amount is sufficient for selective inhibiting ROS1 over TRK (e.g., TRKA, TRKB, and/or TRBC).

As used herein and unless otherwise specified, inhibition of ROS1 includes inhibition of wild type ROS1, or a mutation thereof; inhibition of ALK includes inhibition of wild type ALK, or a mutation thereof; and inhibition of TRK includes inhibition of wild type TRK, or a mutation thereof.

Cancers treated by methods provided herein include, but are not limited to, lung cancer, e.g., non-small cell lung cancer, inflammatory myofibroblastic tumor, ovarian cancer, e.g., serous ovarian carcinoma, melanoma, e.g., spitzoid melanoma, glioblastoma, bile duct cancer, e.g., cholangiocarcinoma, gastric cancer, colorectal cancer, angiosarcoma, anaplastic large cell lymphoma, diffuse large B-cell lymphoma, large B-cell lymphoma, esophageal cancer, e.g., esophageal squamous cell carcinoma, kidney cancer, e.g., renal medullary carcinoma or renal cell carcinoma, breast cancer, e.g., triple negative breast cancer, thyroid cancer, e.g., papillary thyroid cancer, neuroblastoma, epithelioid hemangioendothelioma, colon cancer, and spitzoid tumor.

Cancers treated by methods provided herein include cancers originating from one or more oncogenic proteins selected from ROS1, ALK, TRKA, TRKB, and TRKC. In certain embodiments, cancers treated by methods provided herein include cancers that are drug resistant to treatments directed at one or more oncogenic proteins selected from ROS1, ALK, TRKA, TRKB, and TRKC.

In one embodiment, the cancer in a method provided herein is ROS1 positive (ROS1+). As used herein and unless otherwise specified, a “ROS1 positive” (ROS1+) cancer, disease, or disorder refers to a cancer, disease, or disorder characterized by inappropriately high expression of a ROS1 gene and/or the presence of a mutation in a ROS1 gene. In one embodiment, the mutation alters the biological activity of a ROS1 nucleic acid molecule or polypeptide. As used herein and unless otherwise specified, a “mutation” or “mutant” of ROS1 comprises one or more deletions, substitutions, insertions, inversions, duplications, translocations, or amplifications in the amino acid or nucleotide sequences of ROS1, or fragments thereof. As used herein and unless otherwise specified, a ROS1 “rearrangement” refers to genetic translocations involving the ROS1 gene that may result in ROS1 fusion genes and/or ROS1 fusion proteins. The ROS1 fusion can also include one or more deletions, substitutions, insertions, inversions, duplications, translocations, or amplifications or a fragment thereof, as long as the mutant retains kinase phosphorylation activity.

In one embodiment, the ROS1 mutation comprises one or more ROS1 point mutations. In some embodiments, cancers treated by methods provided herein include one or more mutations in ROS1 kinase. In one embodiment, the one or more ROS1 point mutations are selected from point mutations at E1935, L1947, L1951, G1971, E1974, L1982, S1986, F2004, E2020, L2026, G2032, D2033, C2060, F2075, L2086, V2089, V2098, G2101, D2113, 1981Tins, M2001T, and L2155. In one embodiment, the one or more ROS1 point mutations are selected from G2032R, G2032K, D2033N, S1986F, S1986Y, L2026M, L1951R, E1935G, L1947R, G1971E, E1974K, L1982F, F2004C, F2004V, E2020K, C2060G, F2075V, V2089M, V2098I, G2101A, D2113N, D2113G, L2155S, and L2086F. In one embodiment, the ROS1 mutation is G2032R. In one embodiment, the ROS1 mutation is S1986F. In one embodiment, the ROS1 mutation is S1986Y. In one embodiment, the ROS1 mutation is L2026M. In one embodiment, the ROS1 mutation is D2033N. In one embodiment, the ROS1 mutation is L2086F. In one embodiment, the ROS1 mutation is F2004C. In one embodiment, the ROS1 mutation is F2004V. In one embodiment, the ROS1 mutation is G2101A. In one embodiment, the ROS1 mutation is L1982F. In one embodiment, the ROS1 mutation is co-mutation of G2032R and one or more of S1986F, S1986Y, F2004C, F2004V, L2026M, or D2033N.

In one embodiment, the ROS1 mutation comprises one or more ROS1 rearrangements (in one embodiment, one rearrangement). In one embodiment, the ROS1 mutation comprises one or more ROS1 fusions (in one embodiment, one fusion). In some embodiments, cancers treated by methods provided herein include ROS1 fusions. In one embodiment, the ROS1 fusion is with one of the fusion partners selected from SL (34A2, CD) 74, TPM3, SDC4, EZR, LRIG3, KDELR2, CEP72, CLTL, CTNND2, GOPC (e.g., GOPC-S, GOPC-L), GPRC6A, LIMA1, LRIG3, MSN, MYO5C, OPRM1, SLC6A17 SLMAP, SRSF6, TFG, TMEM106B, TPD52L1, ZCCHC8,CCDC6,CAPRIN1, CEP85L, CHCHD3, CLIP1, EEF1G, KIF21A, KLC1, SART3, ST13, TRIM24, ERC1, FIP1L1, HLAA, KIAA1598, MYO5A, PPFIBP1, PWWP2A, FN1, YWHAE, CCDC30, NCOR2, NFKB2, APOB, PLG, RBP4, and GOLGB1. In one embodiment, the ROS1 fusion is CD74-ROS1 fusion. In one embodiment, the ROS1 fusion is SDC4-ROS1 fusion. In one embodiment, the ROS1 fusion is EZR-ROS1 fusion. In one embodiment, the ROS1 fusion is SLC34A2-ROS1 fusion. In one embodiment, the ROS1 fusion is GOPC-ROS1 fusion (e.g., GOPC—ROS1-S, GOPC-ROS1-L). In one embodiment, the ROS1 fusion is CEP85L-ROS1 fusion.

In one embodiment, the ROS1 mutation comprises one ROS1 rearrangement and one or more ROS1 point mutations. In one embodiment, the ROS1 mutation comprises one or more ROS1 rearrangements from CD74-ROS1, EZR-ROS1, SLC34A2-ROS1, GOPC-ROS1 (e.g., GOPC-ROS1-S, GOPC-ROS1-L), and CEP85L-ROS1, and one or more ROS1 point mutations selected from F2004C, F2004V, and G2032R. In one embodiment, the ROS1 mutation comprises one or more ROS1 rearrangements from CD74-ROS1, EZR-ROS1, and SLC34A2-ROS1, and ROS1 point mutation of G2101A.

In one embodiment, the ROS1 mutation is CD74-ROS1 F2004C. In one embodiment, the ROS1 mutation is CD74-ROS1 F2004V. In one embodiment, the ROS1 mutation is CD74-ROS1 G2101A. In one embodiment, the ROS1 mutation is CD74-ROS1 G2032R. In one embodiment, the ROS1 mutation is CD74-ROS1 S1986F. In one embodiment, the ROS1 mutation is CD74-ROS1 L2026M. In one embodiment, the ROS1 mutation is CD74-ROS1 D2033N. In one embodiment, the ROS1 mutation is EZR-ROS1 F2004C. In one embodiment, the ROS1 mutation is EZR-ROS1 F2004V. In one embodiment, the ROS1 mutation is EZR-ROS1 G2101A. In one embodiment, the ROS1 mutation is EZR-ROS1 G2032R. In one embodiment, the ROS1 mutation is SLC34A2-ROS1 F2004C. In one embodiment, the ROS1 mutation is SLC34A2-ROS1 F2004V. In one embodiment, the ROS1 mutation is SLC34A2-ROS1 G2101A. In one embodiment, the ROS1 mutation is SLC34A2-ROS1 G2032R. In one embodiment, the ROS1 mutation is GOPC-ROS1 F2004C (e.g., GOPC-ROS1-S F2004C, GOPC-ROS1-L F2004C). In one embodiment, the ROS1 mutation is GOPC-ROS1 F2004V (e.g., GOPC-ROS1-S F2004V, GOPC-ROS1-L F2004V). In one embodiment, the ROS1 mutation is GOPC-ROS1 G2032R (e.g., GOPC—ROS1-S G2032R, GOPC-ROS1-L G2032R). In one embodiment, the ROS1 mutation is CEP85L-ROS1 F2004C. In one embodiment, the ROS1 mutation is CEP85L-ROS1 F2004V. In one embodiment, the ROS1 mutation is CEP85L-ROS1 G2032R. In one embodiment, the ROS1 mutation is GOPC-ROS1 L1982F (e.g., GOPC-ROS1-S L1982F, GOPC-ROS1-L L1982F). In one embodiment, the ROS1 mutation is CD74-ROS1 L1982F.

In some embodiments, provided herein is a method of treating a patient population having ROS1 positive solid tumor comprising administering an effective amount of compound 1 or a pharmaceutical composition provided herein to each of the said patient population, wherein the objective response rate (ORR) is at least about 10% (RECIST 1.1) after a cycle of treatment of Compound 1. In one embodiment, the ORR is at least about 20% (RECIST 1.1) after a cycle of treatment of Compound 1. In one embodiment, the ORR is at least about 30% (RECIST 1.1) after a cycle of treatment of Compound 1. In one embodiment, the ORR is at least about 40% (RECIST 1.1) after a cycle of treatment of Compound 1. In one embodiment, the ORR is at least about 50% (RECIST 1.1) after a cycle of treatment of Compound 1. In one embodiment, the ORR is at least about 60% (RECIST 1.1) after a cycle of treatment of Compound 1. In one embodiment, the ORR is at least about 70% (RECIST 1.1) after a cycle of treatment of Compound 1. In one embodiment, the ORR is at least about 80% (RECIST 1.1) after a cycle of treatment of Compound 1. In one embodiment, the ORR is at least about 90% (RECIST 1.1) after a cycle of treatment of Compound 1. In one embodiment, at least about 20% of the patient population achieves partial response. In one embodiment, at least about 30% of the patient population achieves partial response. In one embodiment, at least about 40% of the patient population achieves partial response. In one embodiment, at least about 50% of the patient population achieves partial response. In one embodiment, at least about 60% of the patient population achieves partial response. In one embodiment, at least about 70% of the patient population achieves partial response. In one embodiment, at least about 80% of the patient population achieves partial response. In one embodiment, at least about 10% of the patient population achieves stable disease. In one embodiment, at least about 20% of the patient population achieves stable disease. In one embodiment, at least about 30% of the patient population achieves stable disease. In one embodiment, at least about 40% of the patient population achieves stable disease. In one embodiment, at least about 50% of the patient population achieves stable disease. In one embodiment, at least about 60% of the patient population achieves stable disease. In one embodiment, at least about 70% of the patient population achieves stable disease.

In some embodiments, provided herein is a method of treating a patient population having a solid tumor having ROS1 mutation (e.g., G2032R), comprising administering an effective amount of compound 1 or a pharmaceutical composition provided herein to each of the said patient population, wherein the ORR is at least about 30% (RECIST 1.1) after a cycle of treatment of Compound 1. In one embodiment, the ORR is at least about 40% (RECIST 1.1) after a cycle of treatment of Compound 1. In one embodiment, the ORR is at least about 50% (RECIST 1.1) after a cycle of treatment of Compound 1. In one embodiment, the ORR is at least about 60% (RECIST 1.1) after a cycle of treatment of Compound 1. In one embodiment, the ORR is at least about 70% (RECIST 1.1) after a cycle of treatment of Compound 1. In one embodiment, the ORR is at least about 80% (RECIST 1.1) after a cycle of treatment of Compound 1. In one embodiment, the ORR is at least about 90% (RECIST 1.1) after a cycle of treatment of Compound 1. In one embodiment, at least about 20% of the patient population achieves partial response. In one embodiment, at least about 30% of the patient population achieves partial response. In one embodiment, at least about 40% of the patient population achieves partial response. In one embodiment, at least about 50% of the patient population achieves partial response. In one embodiment, at least about 60% of the patient population achieves partial response. In one embodiment, at least about 70% of the patient population achieves partial response. In one embodiment, at least about 80% of the patient population achieves partial response. In one embodiment, at least about 90% of the patient population achieves partial response. In one embodiment, at least about 5% of the patient population achieves stable disease. In one embodiment, at least about 10% of the patient population achieves stable disease. In one embodiment, at least about 15% of the patient population achieves stable disease. In one embodiment, at least about 20% of the patient population achieves stable disease. In one embodiment, at least about 30% of the patient population achieves stable disease. In one embodiment, at least about 40% of the patient population achieves stable disease. In one embodiment, at least about 50% of the patient population achieves stable disease. In one embodiment, at least about 60% of the patient population achieves stable disease.

In some embodiments, provided herein is a method of treating a patient population having ROS1 positive solid tumor and a CNS disease at baseline comprising administering an effective amount of compound 1 or a pharmaceutical composition provided herein to each of the said patient population, wherein the ORR is at least about 30% (RECIST 1.1) after a cycle of treatment of Compound 1. In one embodiment, the ORR is at least about 40% (RECIST 1.1) after a cycle of treatment of Compound 1. In one embodiment, the ORR is at least about 50% (RECIST 1.1) after a cycle of treatment of Compound 1. In one embodiment, the ORR is at least about 60% (RECIST 1.1) after a cycle of treatment of Compound 1. In one embodiment, the ORR is at least about 70% (RECIST 1.1) after a cycle of treatment of Compound 1. In one embodiment, the ORR is at least about 80% (RECIST 1.1) after a cycle of treatment of Compound 1. In one embodiment, the ORR is at least about 90% (RECIST 1.1) after a cycle of treatment of Compound 1. In one embodiment, at least about 30% of the patient population achieves partial response. In one embodiment, at least about 40% of the patient population achieves partial response. In one embodiment, at least about 50% of the patient population achieves partial response. In one embodiment, at least about 60% of the patient population achieves partial response. In one embodiment, at least about 70% of the patient population achieves partial response. In one embodiment, at least about 80% of the patient population achieves partial response. In one embodiment, at least about 90% of the patient population achieves partial response. In one embodiment, at least about 5% of the patient population achieves stable disease. In one embodiment, at least about 10% of the patient population achieves stable disease. In one embodiment, at least about 15% of the patient population achieves stable disease. In one embodiment, at least about 20% of the patient population achieves stable disease. In one embodiment, at least about 30% of the patient population achieves stable disease. In one embodiment, at least about 40% of the patient population achieves stable disease. In one embodiment, at least about 50% of the patient population achieves stable disease. In one embodiment, at least about 60% of the patient population achieves stable disease.

In some embodiments, provided herein is a method of treating a patient population having ROS1 positive solid tumor after the patient population receives at least two prior ROS1 TKI therapies and at least one chemotherapy, comprising administering an effective amount of compound 1 or a pharmaceutical composition provided herein to each of the said patient population, wherein the ORR is at least about 10% (RECIST 1.1) after a cycle of treatment of Compound 1. In one embodiment, the ORR is at least about 20% (RECIST 1.1) after a cycle of treatment of Compound 1. In one embodiment, the ORR is at least about 30% (RECIST 1.1) after a cycle of treatment of Compound 1. In one embodiment, the ORR is at least about 40% (RECIST 1.1) after a cycle of treatment of Compound 1. In one embodiment, the ORR is at least about 50% (RECIST 1.1) after a cycle of treatment of Compound 1. In one embodiment, the ORR is at least about 60% (RECIST 1.1) after a cycle of treatment of Compound 1. In one embodiment, the ORR is at least about 70% (RECIST 1.1) after a cycle of treatment of Compound 1. In one embodiment, the ORR is at least about 80% (RECIST 1.1) after a cycle of treatment of Compound 1. In one embodiment, the ORR is at least about 90% (RECIST 1.1) after a cycle of treatment of Compound 1. In one embodiment, at least about 20% of the patient population achieves partial response. In one embodiment, at least about 30% of the patient population achieves partial response. In one embodiment, at least about 40% of the patient population achieves partial response. In one embodiment, at least about 50% of the patient population achieves partial response. In one embodiment, at least about 60% of the patient population achieves partial response. In one embodiment, at least about 70% of the patient population achieves partial response. In one embodiment, at least about 80% of the patient population achieves partial response. In one embodiment, at least about 90% of the patient population achieves partial response. In one embodiment, at least about 10% of the patient population achieves stable disease. achieves stable disease. In one embodiment, at least about 20% of the patient population achieves stable disease. In one embodiment, at least about 30% of the patient population achieves stable disease. In one embodiment, at least about 40% of the patient population achieves stable disease. In one embodiment, at least about 50% of the patient population achieves stable disease. In one embodiment, at least about 60% of the patient population achieves stable disease. In one embodiment, at least about 70% of the patient population achieves stable disease. In one embodiment, at least about 80% of the patient population achieves stable disease.

In some embodiments, provided herein is a method of treating a patient population having ROS1 positive solid tumor after the patient population receives at least one prior ROS1 TKI therapies and at least one chemotherapy, comprising administering an effective amount of compound 1 or a pharmaceutical composition provided herein to each of the said patient population, wherein the ORR is at least about 10% (RECIST 1.1) after a cycle of treatment of Compound 1. In one embodiment, the ORR is at least about 20% (RECIST 1.1) after a cycle of treatment of Compound 1. In one embodiment, the ORR is at least about 30% (RECIST 1.1) after a cycle of treatment of Compound 1. In one embodiment, the ORR is at least about 40% (RECIST 1.1) after a cycle of treatment of Compound 1. In one embodiment, the ORR is at least about 50% (RECIST 1.1) after a cycle of treatment of Compound 1. In one embodiment, the ORR is at least about 60% (RECIST 1.1) after a cycle of treatment of Compound 1. In one embodiment, the ORR is at least about 70% (RECIST 1.1) after a cycle of treatment of Compound 1. In one embodiment, the ORR is at least about 80% (RECIST 1.1) after a cycle of treatment of Compound 1. In one embodiment, the ORR is at least about 90% (RECIST 1.1) after a cycle of treatment of Compound 1. In one embodiment, at least about 20% of the patient population achieves partial response. In one embodiment, at least about 30% of the patient population achieves partial response. In one embodiment, at least about 40% of the patient population achieves partial response. In one embodiment, at least about 50% of the patient population achieves partial response. In one embodiment, at least about 60% of the patient population achieves partial response. In one embodiment, at least about 70% of the patient population achieves partial response. In one embodiment, at least about 80% of the patient population achieves partial response. In one embodiment, at least about 90% of the patient population achieves partial response. In one embodiment, at least about 10% of the patient population achieves stable disease. achieves stable disease. In one embodiment, at least about 20% of the patient population achieves stable disease. In one embodiment, at least about 30% of the patient population achieves stable disease. In one embodiment, at least about 40% of the patient population achieves stable disease. In one embodiment, at least about 50% of the patient population achieves stable disease. In one embodiment, at least about 60% of the patient population achieves stable disease. In one embodiment, at least about 70% of the patient population achieves stable disease. In one embodiment, at least about 80% of the patient population achieves stable disease.

In some embodiments, provided herein is a method of treating a patient population having ROS1 positive solid tumor after the patient population receives prior treatment of lorlatinib and/or repotrectinib, comprising administering an effective amount of compound 1 or a pharmaceutical composition provided herein to each of the said patient population, wherein the ORR is at least about 10% (RECIST 1.1) after a cycle of treatment of Compound 1. In one embodiment, the ORR is at least about 20% (RECIST 1.1) after a cycle of treatment of Compound 1. In one embodiment, the ORR is at least about 30% (RECIST 1.1) after a cycle of treatment of Compound 1. In one embodiment, the ORR is at least about 40% (RECIST 1.1) after a cycle of treatment of Compound 1. In one embodiment, the ORR is at least about 50% (RECIST 1.1) after a cycle of treatment of Compound 1. In one embodiment, the ORR is at least about 60% (RECIST 1.1) after a cycle of treatment of Compound 1. In one embodiment, the ORR is at least about 70% (RECIST 1.1) after a cycle of treatment of Compound 1. In one embodiment, the ORR is at least about 80% (RECIST 1.1) after a cycle of treatment of Compound 1. In one embodiment, the ORR is at least about 90% (RECIST 1.1) after a cycle of treatment of Compound 1. In one embodiment, at least about 20% of the patient population achieves partial response. In one embodiment, at least about 30% of the patient population achieves partial response. In one embodiment, at least about 40% of the patient population achieves partial response. In one embodiment, at least about 50% of the patient population achieves partial response. In one embodiment, at least about 60% of the patient population achieves partial response. In one embodiment, at least about 70% of the patient population achieves partial response. In one embodiment, at least about 80% of the patient population achieves partial response. In one embodiment, at least about 90% of the patient population achieves partial response. In one embodiment, at least about 10% of the patient population achieves stable disease. achieves stable disease. In one embodiment, at least about 10% of the patient population achieves stable disease. In one embodiment, at least about 20% of the patient population achieves stable disease. In one embodiment, at least about 30% of the patient population achieves stable disease. In one embodiment, at least about 40% of the patient population achieves stable disease. In one embodiment, at least about 50% of the patient population achieves stable disease. In one embodiment, at least about 60% of the patient population achieves stable disease. In one embodiment, at least about 70% of the patient population achieves stable disease. In one embodiment, at least about 80% of the patient population achieves stable disease.

In one embodiment, the ROS1+ cancer is determined by an FDA-approved test or other tests known in the art. The tests that can be used include, e.g., Oncomine™ Dx Target Test by Thermo Fisher Scientific. (a qualitative in vitro diagnostic test that uses targeted high-throughput, parallel-sequencing technology to detect sequence variations in 23 genes in DNA and RNA isolated from formalin-fixed, paraffin-embedded tumor (FFPE) tissue samples from patients with non-small cell lung cancer (NSCLC) using the Ion PGM Dx System); Vysis ROS1 Break Apart FISH Probe Kit (a qualitative test to detect rearrangements involving ROS1 gene rearrangements at 6q22 via fluorescence in situ hybridization (FISH) in formalin-fixed, paraffin-embedded (FFPE) non-small cell lung cancer (NSCLC) tissue specimens) or Reverse transcription-Polymerase Chain Reaction (RT-PCR) or Next Generation Sequencing (NGS) via a local diagnostic test.

Also provided are methods of treating a subject having a cancer (e.g., a ROS1 positive cancer) that include: determining whether a cancer cell in a sample obtained from a subject having a cancer and previously administered a first ROS1 inhibitor, has one or more ROS1 inhibitor resistance mutations; and administering a compound of Formula (I) or a pharmaceutically acceptable salt or solvate thereof as a monotherapy or in conjunction with another anticancer agent to the subject if the subject has a cancer cell that has one or more ROS1 inhibitor resistance mutations. In some embodiments, the one or more ROS1 inhibitor resistance mutations confer increased resistance to a cancer cell or tumor to treatment with the first ROS1 inhibitor. In some embodiments, the one or more ROS1 inhibitor resistance mutations include one or more ROS1 inhibitor resistance mutations. For example, the one or more ROS1 inhibitor resistance mutations can include a substitution at one or more of amino acid positions 2032, 2033, 1986, 2026, 1951, 1935, 1947, 1971, 1974, 1982, 2004, 2020, 2060, 2075, 2089, 2098, 2101, 2113, 2155, 2032, and 2086, e.g., G2032R, D2033N, S1986F, S1986Y, L2026M, L1951R, E1935G, L1947R, G1971E, E1974K, L1982F, F2004C, F2004V, E2020K, C2060G, F2075V, V2089M, V2098I, G2101A, D2113N, D2113G, L2155S, L2032K, and L2086F. In some embodiments, another anticancer agent is any anticancer agent known in the art. For example, another anticancer agent can be another ROS1 inhibitor (e.g., a second ROS1 inhibitor).

In one embodiment, a compound provided herein is a CNS-penetrating compound. In one embodiment, after the administration of an effective amount of a compound provided herein (e.g., orally or intravenously), the compound is able to penetrate CNS (e.g., blood-brain barrier) and achieve a concentration in CNS (e.g., brain) that is still sufficient to inhibit (e.g., selectively inhibit) ROS1.

In one embodiment, provided herein is a method for treating CNS metastases of a cancer, comprising administering to a subject in need thereof an effective amount of a compound provided herein, e.g., a compound of Formula (I), or a stereoisomer, or a mixture of stereoisomers thereof, or a pharmaceutically acceptable salt thereof. In one embodiment, the CNS metastases is brain metastases. In one embodiment, the cancer is a ROS1+ cancer.

In some embodiments, the compound is an inhibitor of human tropomyosin receptor kinase A, B, or C. In certain embodiments, the IC₅₀ of the compound for inhibition of mutant or non-mutant ROS1 or ALK is no more than one-fifth of the IC₅₀ of the compound for inhibition of wild-type tropomyosin receptor kinase A, B, or C. TRK inhibition, particularly in the central nervous system (CNS), has been associated with adverse reactions, including dizziness/ataxia/gait disturbance, paraesthesia, weight gain and cognitive changes.

In some embodiments, provided is a method of minimizing treatment-related adverse events in a subject in need of treatment for cancer (e.g., a ROS1 positive cancer), the method comprising administering to the subject a therapeutically effective amount of a compound provided herein, e.g., a compound of Formula (I), a stereoisomer, or a mixture of stereoisomers thereof, or a pharmaceutically acceptable salt thereof, and wherein the method minimizes treatment-related adverse events associated with TRK inhibitors. In some embodiments, the cancer is a ROS1-associated cancer. In some embodiments, the adverse events are one or more of TRK-related CNS adverse events.

As used herein “minimizing” adverse events refers to a reduction in the incidence of adverse events in a subject or patient population compared to the paradigmatic incidence of adverse events in a subject or patient population treated with TRK inhibitors (e.g., entrectinib, repotrectinib, or lorlatinib). In some embodiments, the incidence of an adverse event refers to the frequency or percentage of a specific adverse event over a subject or patient population. In some embodiments, the incidence of an adverse event refers to the total number of adverse events experienced by an individual subject. In some embodiments, minimizing adverse events refers to minimizing TRK-related CNS adverse events. In some embodiments, minimizing TRK-related CNS adverse events means less than 40% of the patient population has a TRK-related CNS adverse event. In some embodiments, minimizing TRK-related CNS adverse events means less than 35%, less than 30%, less than 25%, less than 20%, less than 15%, less than 10% or less than 5% of the patient population has a TRK-related CNS adverse event. In some embodiments, minimizing TRK-related CNS adverse events means less than 12% of the patient population have more than one TRK-related CNS adverse event. In some embodiments, minimizing TRK-related CNS adverse events means less than 11%, less than 10%, less than 9%, less than 8%, less than 7%, less than 6%, less than 5%, less than 4%, or less than 3% of the patient population have more than one TRK-related CNS adverse event.

In some embodiments, TRK-related CNS treatment-related adverse events refers to one or more of the following: dizziness, ataxia, gait disturbance, paraesthesia, weight gain, hyperphagia, paresthesias, abnormal movement, cognitive changes, speech effects (e.g., dysarthria, slow speech, or speech disorder), mood disorder (e.g., irritability, anxiety, depression, affect lability, personality change, mood swings, affective disorder, aggression, agitation, mood altered, depressed mood, euphoric mood, or mania), and cognitive disorder (e.g., memory impairment, cognitive disorder, amnesia, confusion, disturbance in attention, delirium, mental impairment, attention deficit/hyperactivity disorder, dementia, sleep disturbance, or reading disorder).

In one embodiment, provided herein is a method for preventing or limiting TRK-related treatment-related CNS side effect or adverse event in a cancer treatment, comprising administering to a subject in need thereof an effective amount of a compound provided herein, e.g., a compound of Formula (I), or a stereoisomer, or a mixture of stereoisomers thereof, or a pharmaceutically acceptable salt thereof. In one embodiment, the method prevents the occurrence of the TRK-related CNS adverse event. In one embodiment, the method limits the frequency of occurrence of the TRK-related CNS adverse event. In one embodiment, the method limits the severity of the TRK-related side effect. In one embodiment, provided herein is a method for treating CNS metastases of a cancer with reduced TRK-related side effect, comprising administering to a subject in need thereof an effective amount of a compound provided herein, e.g., a compound of Formula (I), or a stereoisomer, or a mixture of stereoisomers thereof, or a pharmaceutically acceptable salt thereof. In one embodiment, the reduction/limiting/prevention in CNS side effect or adverse event is determined in a statistical sample, as compared to a standard of care treatment, e.g., an approved ROS1 and/or ALK inhibitor (e.g., crizotinib, entrectinib, lorlatinib, or repotrectinib) for ROS1+ and/or ALK+ cancer. In one embodiment, the TRK-related side effect is a TRKB-related CNS side effect. In one embodiment, the TRK-related CNS side effect or adverse event is dizziness, ataxia, gait disturbance, paraesthesia, weight gain, cognitive impairment, a mood disorder, or sleep disturbance.

In one embodiment, provided herein is a method for treating cancer, comprising administering to a subject in need thereof a therapeutically effective amount of a compound provided herein, e.g., a compound of Formula (I), or a stereoisomer, or a mixture of stereoisomers thereof, or a pharmaceutically acceptable salt thereof. In one embodiment, the cancer is a ROS1-associated cancer. In one embodiment, the cancer is a ROS1+ cancer. In one embodiment, the cancer is identified to be ROS1

In one embodiment, provided herein is a method for treating a ROS1+ cancer, comprising administering to a subject in need thereof a therapeutically effective amount of a compound provided herein, e.g., a compound of Formula (I), or a stereoisomer, or a mixture of stereoisomers thereof, or a pharmaceutically acceptable salt thereof.

In one embodiment, provided herein is a method for treating cancer in a subject, comprising: (i) identifying the cancer in the subject to be ROS1+, and (ii) administering to the subject a therapeutically effective amount of a compound provided herein, e.g., a compound of Formula (I), or a stereoisomer, or a mixture of stereoisomers thereof, or a pharmaceutically acceptable salt thereof.

In one embodiment, the cancer (or ROS1+ cancer) is a solid tumor. In one embodiment, the cancer (or ROS1+ cancer) is an advanced solid tumor. In one embodiment, the cancer (or ROS1+ cancer) is a locally advanced solid tumor. In one embodiment, the cancer (or ROS1+ cancer) is lung cancer, e.g., non-small cell lung cancer (NSCLC) or bronchus cancer, glioblastoma, inflammatory myofibroblastic tumor (IMT), bile duct cancer, e.g., cholangiocarcinoma, ovarian cancer, e.g., serous ovarian carcinoma, gastric cancer, colorectal cancer, angiosarcoma, melanoma, e.g., spitzoid melanoma, epithelioid hemangioendothelioma, esophageal cancer, e.g., esophageal squamous cell carcinoma (ESCC), kidney cancer, e.g., renal medullary carcinoma or renal cell carcinoma, breast cancer, e.g., triple negative breast cancer, colon cancer, thyroid cancer, e.g., papillary thyroid cancer, spitzoid tumor, pancreatic cancer, inflammatory hepatocellular adenoma, or neuroblastoma.

In one embodiment, the cancer is lung cancer. In one embodiment, the cancer is non-small cell lung cancer. In one embodiment, the cancer is ROS1+ non-small cell lung cancer. In one embodiment, the cancer is relapsed or refractory non-small cell lung cancer. In one embodiment, the cancer is relapsed or refractory ROS1+ non-small cell lung cancer. In one embodiment, the cancer is newly diagnosed non-small cell lung cancer. In one embodiment, the cancer is newly diagnosed ROS1+ non-small cell lung cancer. In one embodiment, the cancer is bronchus cancer. In one embodiment, the cancer is ROS1+ bronchus cancer. In one embodiment, the cancer is relapsed or refractory bronchus cancer. In one embodiment, the cancer is relapsed or refractory ROS1+ bronchus cancer. In one embodiment, the cancer is newly diagnosed bronchus cancer. In one embodiment, the cancer is newly diagnosed ROS1+ bronchus cancer.

In one embodiment, the cancer is glioblastoma. In one embodiment, the cancer is relapsed or refractory glioblastoma. In one embodiment, the cancer is relapsed or refractory ROS1+ glioblastoma. In one embodiment, the cancer is newly diagnosed glioblastoma. In one embodiment, the cancer is newly diagnosed ROS1+ glioblastoma.

In one embodiment, the cancer is IMT. In one embodiment, the cancer is ROS1+ IMT. In one embodiment, the cancer is relapsed or refractory IMT. In one embodiment, the cancer is relapsed or refractory ROS1+ IMT. In one embodiment, the cancer is newly diagnosed IMT. In one embodiment, the cancer is newly diagnosed ROS1+ IMT.

In one embodiment, the cancer is bile duct cancer. In one embodiment, the cancer is cholangiocarcinoma. In one embodiment, the cancer is ROS1+ cholangiocarcinoma. In one embodiment, the cancer is relapsed or refractory cholangiocarcinoma. In one embodiment, the cancer is relapsed or refractory ROS1+ cholangiocarcinoma. In one embodiment, the cancer is newly diagnosed cholangiocarcinoma. In one embodiment, the cancer is newly diagnosed ROS1+ cholangiocarcinoma.

In one embodiment, the cancer is ovarian cancer. In one embodiment, the cancer is ROS1+ ovarian cancer. In one embodiment, the cancer is relapsed or refractory ovarian cancer. In one embodiment, the cancer is relapsed or refractory ROS1+ ovarian cancer. In one embodiment, the cancer is newly diagnosed ovarian cancer. In one embodiment, the cancer is newly diagnosed ROS1+ ovarian cancer. In one embodiment, the ovarian cancer is serous ovarian carcinoma. In one embodiment, the ovarian cancer is high grade serous ovarian carcinoma.

In one embodiment, the cancer is gastric cancer. In one embodiment, the cancer is ROS1+ gastric cancer. In one embodiment, the cancer is relapsed or refractory gastric cancer. In one embodiment, the cancer is relapsed or refractory ROS1+ gastric cancer. In one embodiment, the cancer is newly diagnosed gastric cancer. In one embodiment, the cancer is newly diagnosed ROS1+ gastric cancer.

In one embodiment, the cancer is colorectal cancer. In one embodiment, the cancer is ROS1+ colorectal cancer. In one embodiment, the cancer is relapsed or refractory colorectal cancer. In one embodiment, the cancer is relapsed or refractory ROS1+ colorectal cancer. In one embodiment, the cancer is newly diagnosed colorectal cancer. In one embodiment, the cancer is newly diagnosed ROS1+ colorectal cancer.

In one embodiment, the cancer is angiosarcoma. In one embodiment, the cancer is ROS1+ angiosarcoma. In one embodiment, the cancer is relapsed or refractory angiosarcoma. In one embodiment, the cancer is relapsed or refractory ROS1+ angiosarcoma. In one embodiment, the cancer is newly diagnosed angiosarcoma. In one embodiment, the cancer is newly diagnosed ROS1+ angiosarcoma.

In one embodiment, the cancer is melanoma. In one embodiment, the cancer is spitzoid tumor. In one embodiment, the cancer is spitzoid melanoma. In one embodiment, the cancer is ROS1+ spitzoid melanoma. In one embodiment, the cancer is relapsed or refractory spitzoid melanoma. In one embodiment, the cancer is relapsed or refractory ROS1+ spitzoid melanoma. In one embodiment, the cancer is newly diagnosed spitzoid melanoma. In one embodiment, the cancer is newly diagnosed ROS1+ spitzoid melanoma.

In one embodiment, the cancer is epithelioid hemangioendothelioma. In one embodiment, the cancer is ROS1+ epithelioid hemangioendothelioma. In one embodiment, the cancer is relapsed or refractory epithelioid hemangioendothelioma. In one embodiment, the cancer is relapsed or refractory ROS1+ epithelioid hemangioendothelioma. In one embodiment, the cancer is newly diagnosed epithelioid hemangioendothelioma. In one embodiment, the cancer is newly diagnosed ROS1+ epithelioid hemangioendothelioma.

In one embodiment, the cancer is esophageal cancer. In one embodiment, the cancer is ESCC. In one embodiment, the cancer is ROS1+ ESCC. In one embodiment, the cancer is relapsed or refractory ESCC. In one embodiment, the cancer is relapsed or refractory ROS1+ ESCC. In one embodiment, the cancer is newly diagnosed ESCC. In one embodiment, the cancer is newly diagnosed ROS1+ ESCC.

In one embodiment, the cancer is kidney cancer. In one embodiment, the cancer is renal medullary carcinoma. In one embodiment, the cancer is ROS1+ renal medullary carcinoma. In one embodiment, the cancer is relapsed or refractory renal medullary carcinoma. In one embodiment, the cancer is relapsed or refractory ROS1+ renal medullary carcinoma. In one embodiment, the cancer is newly diagnosed renal medullary carcinoma. In one embodiment, the cancer is newly diagnosed ROS1+ renal medullary carcinoma. In one embodiment, the cancer is renal cell carcinoma. In one embodiment, the cancer is ROS1+ renal cell carcinoma. In one embodiment, the cancer is relapsed or refractory renal cell carcinoma. In one embodiment, the cancer is relapsed or refractory ROS1+ renal cell carcinoma. In one embodiment, the cancer is newly diagnosed renal cell carcinoma. In one embodiment, the cancer is newly diagnosed ROS1+ renal cell carcinoma.

In one embodiment, the cancer is breast cancer. In one embodiment, the cancer is ROS1+ breast cancer. In one embodiment, the cancer is relapsed or refractory breast cancer. In one embodiment, the cancer is relapsed or refractory ROS1+ breast cancer. In one embodiment, the cancer is newly diagnosed breast cancer. In one embodiment, the cancer is newly diagnosed ROS1+ breast cancer. In one embodiment, the breast cancer is triple negative breast cancer.

In one embodiment, the cancer is colon cancer. In one embodiment, the cancer is ROS1+ colon cancer. In one embodiment, the cancer is relapsed or refractory colon cancer. In one embodiment, the cancer is relapsed or refractory ROS1+ colon cancer. In one embodiment, the cancer is newly diagnosed colon cancer. In one embodiment, the cancer is newly diagnosed ROS1+ colon cancer.

In one embodiment, the cancer is thyroid cancer. In one embodiment, the cancer is papillary thyroid cancer. In one embodiment, the cancer is ROS1+ papillary thyroid cancer. In one embodiment, the cancer is relapsed or refractory papillary thyroid cancer. In one embodiment, the cancer is relapsed or refractory ROS1+ papillary thyroid cancer. In one embodiment, the cancer is newly diagnosed papillary thyroid cancer. In one embodiment, the cancer is newly diagnosed ROS1+ papillary thyroid cancer.

In one embodiment, the cancer is ROS1+ glioma (e.g. Grade 1, Grade 2, Grade 3, or Grade 4). In one embodiment, the cancer is relapsed or refractory glioma. In one embodiment, the cancer is relapsed or refractory ROS1+ glioma. In one embodiment, the cancer is newly diagnosed ROS1+ glioma. In one embodiment, the cancer is ROS1+ glioblastoma. In one embodiment, the cancer is newly diagnosed ROS1+ glioblastoma. In one embodiment, the cancer is relapsed or refractory glioblastoma. In one embodiment, the cancer is relapsed or refractory ROS1+ glioblastoma. In one embodiment, the cancer is neuroblastoma. In one embodiment, the cancer is ROS1+ neuroblastoma. In one embodiment, the cancer is relapsed or refractory neuroblastoma. In one embodiment, the cancer is relapsed or refractory ROS1+ neuroblastoma. one embodiment, the cancer is newly diagnosed neuroblastoma. In one embodiment, the cancer is newly diagnosed ROS1+ neuroblastoma.

In one embodiment, the cancer is ROS1+ pancreatic cancer. In one embodiment, the cancer is relapsed or refractory pancreatic cancer. In one embodiment, the cancer is relapsed or refractory ROS1+ pancreatic cancer. one embodiment, the cancer is newly diagnosed neuroblastoma. In one embodiment, the cancer is newly diagnosed ROS1+ pancreatic cancer.

In one embodiment, the cancer is ROS1+ inflammatory hepatocellular adenoma. In one embodiment, the cancer is relapsed or refractory inflammatory hepatocellular adenoma. In one embodiment, the cancer is relapsed or refractory ROS1+ inflammatory hepatocellular adenoma. one embodiment, the cancer is newly diagnosed neuroblastoma. In one embodiment, the cancer is newly diagnosed ROS1+ inflammatory hepatocellular adenoma.

In one embodiment, the cancer (or ROS1+ cancer, or ALK+ cancer) is a hematological cancer. In one embodiment, the cancer (or ROS1+ cancer, or ALK+ cancer) is lymphoma. In one embodiment, the lymphoma is non-Hodgkin lymphoma. In one embodiment, the lymphoma is anaplastic large cell lymphoma (ALCL), diffuse large B-cell lymphoma (DLBCL), or large B-cell lymphoma. In addition to hematological cancer, methods for treating other blood disorder or hematologic malignancy that is ROS1+ or ALK+ are also provided herein.

In one embodiment, the cancer is ALCL. In one embodiment, the cancer is ROS1+ ALCL. In one embodiment, the cancer is ALK+ ALCL. In one embodiment, the cancer is relapsed or refractory ALCL. In one embodiment, the cancer is relapsed or refractory ROS1+ ALCL. In one embodiment, the cancer is relapsed or refractory ALK+ ALCL. In one embodiment, the cancer is newly diagnosed ALCL. In one embodiment, the cancer is newly diagnosed ROS1+ ALCL. In one embodiment, the cancer is newly diagnosed ALK+ ALCL.

In one embodiment, the cancer is DLBCL. In one embodiment, the cancer is ROS1+ DLBCL. In one embodiment, the cancer is ALK+ DLBCL. In one embodiment, the cancer is relapsed or refractory DLBCL. In one embodiment, the cancer is relapsed or refractory ROS1+ DLBCL. In one embodiment, the cancer is relapsed or refractory ALK+ DLBCL. In one embodiment, the cancer is newly diagnosed DLBCL. In one embodiment, the cancer is newly diagnosed ROS1+ DLBCL. In one embodiment, the cancer is newly diagnosed ALK+ DLBCL.

In one embodiment, the cancer is large B-cell lymphoma. In one embodiment, the cancer is ROS1+ large B-cell lymphoma. In one embodiment, the cancer is ALK+ large B-cell lymphoma. In one embodiment, the cancer is relapsed or refractory large B-cell lymphoma. In one embodiment, the cancer is relapsed or refractory ROS1+ large B-cell lymphoma. In one embodiment, the cancer is relapsed or refractory ALK+ large B-cell lymphoma. In one embodiment, the cancer is newly diagnosed large B-cell lymphoma. In one embodiment, the cancer is newly diagnosed ROS1+ large B-cell lymphoma. In one embodiment, the cancer is newly diagnosed ALK+ large B-cell lymphoma. In one embodiment, the cancer (or ROS1+ cancer) is new diagnosed. In one embodiment, the cancer (or ROS1+ cancer) is previously untreated.

In one embodiment, the cancer (or ROS1+ cancer) is relapsed or refractory. In one embodiment, the cancer is relapsed. In one embodiment, the cancer (or ROS1+ cancer) is refractory.

In one embodiment, the subject is previously untreated. In one embodiment, the subject is treatment naïve to tyrosine kinase inhibitor (TKI) therapy. In one embodiment, the subject has received one or more prior lines of therapy. In one embodiment, the subject has received two or more prior lines of therapy. In one embodiment, the subject has developed resistance to one or more of the prior lines of therapy. In one embodiment, the prior therapy comprises a tyrosine kinase inhibitor (TKI). In one embodiment, the prior TKI therapy comprises a treatment with one or more of crizotinib, ceritinib, alectinib, brigatinib, lorlatinib, entrectinib, repotrectinib, cabozantinib, foretinib, taletrectinib, merestinib, masitinib, and ensartinib. In one embodiment, the prior therapy comprises one or more chemotherapies. In one embodiment, the one or more chemotherapies are in addition to the TKI therapy.

In one embodiment, the cancer is advanced cancer, e.g. relapsed after, refractory to, or resistant to the prior treatment by a TKI.

In one embodiment, the cancer (or ROS1+ cancer) is resistant to a tyrosine kinase inhibitor (TKI).

In one embodiment, the cancer is resistant lung cancer. In one embodiment, the cancer is resistant bronchus cancer. In one embodiment, the cancer is resistant non-small cell lung cancer. In one embodiment, the cancer is non-small cell lung cancer resistant to a TKI. In one embodiment, the cancer is ROS1+ non-small cell lung cancer resistant to a TKI.

In one embodiment, the cancer is lung cancer (e.g., NSCLC, bronchus cancer). In one embodiment, the cancer is advanced lung cancer, e.g. relapsed after, or refractory to, prior treatment by a TKI.

In one embodiment, a compound provided herein is administered as first-line treatment. In one embodiment, a compound provided herein is administered as second-line treatment. In one embodiment, a compound provided herein is administered as third or fourth-line treatment.

In one embodiment, the cancer (or ROS1+ cancer) is metastatic. In one embodiment, the cancer has CNS metastases. In one embodiment, the cancer has brain metastases. In one embodiment, the cancer is metastatic non-small cell lung cancer (NSCLC). In one embodiment, the cancer is metastatic ROS1+NSCLC.

In one embodiment, provided herein is a method for treating a patient with metastatic ROS1+ non-small cell lung cancer (NSCLC), comprising administering to the patient a therapeutically effective amount of a compound provided herein, e.g., a compound of Formula (I), or a stereoisomer, or a mixture of stereoisomers thereof, or a pharmaceutically acceptable salt thereof.

In one embodiment, the patient is an adult patient. In one embodiment, the patient is a pediatric patient.

In one embodiment, provided herein is a method for treating an adult patient with metastatic ROS1+NSCLC, comprising administering to the patient a therapeutically effective amount of a compound provided herein, e.g., a compound of Formula (I), or a stereoisomer, or a mixture of stereoisomers thereof, or a pharmaceutically acceptable salt thereof.

In one embodiment, provided herein is a method for treating an adult patient with metastatic ROS1+NSCLC, comprising administering to the patient a therapeutically effective amount of a compound provided herein, e.g., a compound of Formula (I), or a stereoisomer, or a mixture of stereoisomers thereof, or a pharmaceutically acceptable salt thereof, wherein the patient has progressed on or is intolerant of at least 1 prior TKI therapy.

In one embodiment, provided herein is a method for treating an adult patient with metastatic NSCLC that is ROS1+ with solvent front mutation G2032R, comprising administering to the patient a therapeutically effective amount of a compound provided herein, e.g., a compound of Formula (I), or a stereoisomer, or a mixture of stereoisomers thereof, or a pharmaceutically acceptable salt thereof, wherein the patient has progressed on or is intolerant of at least 1 prior TKI therapy.

In one embodiment, provided herein is a method for treating a ROS1-associated (or ROS1+) cancer in a subject in need thereof, wherein the cancer has developed resistance to a tyrosine kinase inhibitor (TKI), the method comprising administering to the subject a therapeutically effective amount of a compound provided herein, e.g., a compound of Formula (I), or a stereoisomer, or a mixture of stereoisomers thereof, or a pharmaceutically acceptable salt thereof.

In one embodiment, provided herein is a method for treating a ROS1-associated (or ROS1+) cancer in a subject in need thereof, wherein the cancer has developed resistance to a tyrosine kinase inhibitor (TKI), and wherein the cancer has been identified as having one or more ROS1 inhibitor resistance mutations, the method comprising administering to the subject a therapeutically effective amount of a compound provided herein, e.g., a compound of Formula (I), or a stereoisomer, or a mixture of stereoisomers thereof, or a pharmaceutically acceptable salt thereof. In one embodiment, the one or more ROS1 inhibitor resistance mutations comprise one or more amino acid substitutions at an amino acid position selected from 1986, 2004, 2026, 2032, and 2033. In one embodiment, the one or more ROS1 inhibitor resistance mutations comprise one or more amino acid substitutions selected from S1986F, S1986Y, F2004C, F2004V, L2026M, G2032R, D2033N, L2086F, and G2101A. In one embodiment, the one or more ROS1 inhibitor resistance mutations is G2032R. In one embodiment, the one or more ROS1 inhibitor resistance mutations comprise G2032R and one or more of S1986F, S1986Y, F2004C, F2004V, L2026M, D2033N, or G2101A. In one embodiment, the ROS1 inhibitor resistance mutation is L2086F.

In one embodiment, the TKI is a ROS1 inhibitor. In one embodiment, the TKI is an ALK inhibitor. In one embodiment, the TKI is crizotinib, ceritinib, alectinib, brigatinib, lorlatinib, entrectinib, repotrectinib, cabozantinib, foretinib, merestinib, taletrectinib, masitinib, or ensartinib. In one embodiment, the TKI is crizotinib. In one embodiment, the TKI is entrectinib. In one embodiment, the TKI is lorlatinib. In one embodiment, the TKI is repotrectinib.

In certain embodiments, the subject has relapsed after first-line treatment of the cancer. In other embodiments, the subject has relapsed after second-line treatment of the cancer.

In certain embodiments, the methods for treating or preventing cancer can be demonstrated by one or more responses such as increased apoptosis, inhibition of tumor growth, reduction of tumor metastasis, inhibition of tumor metastasis, reduction of microvessel density, decreased neovascularization, inhibition of tumor migration, tumor regression, and increased survival of the subject.

### Combination Treatments

In some embodiments, the method of treating or preventing cancer may comprise administering a compound of Formula (I) conjointly with one or more other chemotherapeutic agent(s).

As used herein and unless otherwise specified, by “conjointly” or “in combination with”, it is not intended to imply that the other agent and the compound of Formula (I) must be administered at the same time and/or formulated for delivery together, although these methods of delivery are within the scope of this disclosure. The compound provided herein can be administered concurrently with, prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before), or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after), one or more other agents (e.g., one or more other additional agents). In general, each therapeutic agent is administered at a dose and/or on a time schedule determined for that particular agent. The other therapeutic agent can be administered with the compound provided herein in a single composition or separately in a different composition. Triple therapy is also contemplated herein. Chemotherapeutic agents that may be conjointly administered with compounds provided herein include: 1-amino-4-phenylamino-9,10-dioxo-9,10-dihydroanthracene-2-sulfonate (acid blue 25), 1-amino-4-[4-hydroxyphenyl-amino]-9,10-dioxo-9,10-dihydroanthracene-2-sulfonate, 1-amino-4-[4-aminophenylamino]-9,10-dioxo-9,10-dihydroanthracene-2-sulfonate, 1-amino-4-[1-naphthylamino]-9,10-dioxo-9,10-dihydroanthracene-2-sulfonate, 1-amino-4-[4-fluoro-2-carboxyphenylamino]-9,10-dioxo-9,10-dihydroanthracene-2-sulfonate, 1-amino-4-[2-anthracenylamino]-9,10-dioxo-9,10-dihydroanthracene-2-sulfonate, ABT-263, afatinib dimaleate, axitinib, aminoglutethimide, amsacrine, anastrozole, APCP, asparaginase, AZD5363, Bacillus Calmette-Guérin vaccine (bcg), bicalutamide, bleomycin, bortezomib, β-methylene-ADP (AOPCP), buserelin, busulfan, cabazitaxel, cabozantinib, campothecin, capecitabine, carboplatin, carfilzomib, carmustine, ceritinib, chlorambucil, chloroquine, cisplatin, cladribine, clodronate, cobimetinib, colchicine, crizotinib, cyclophosphamide, cyproterone, cytarabine, dacarbazine, dactinomycin, daunorubicin, demethoxyviridin, dexamethasone, dichloroacetate, dienestrol, diethylstilbestrol, docetaxel, doxorubicin, epirubicin, eribulin, erlotinib, estradiol, estramustine, etoposide, everolimus, exemestane, filgrastim, fludarabine, fludrocortisone, fluorouracil, fluoxymesterone, flutamide, gefitinib, gemcitabine, genistein, goserelin, GSK1120212, hydroxyurea, idarubicin, ifosfamide, imatinib, interferon, irinotecan, ixabepilone, lenalidomide, letrozole, leucovorin, leuprolide, levamisole, lomustine, lonidamine, mechlorethamine, medroxyprogesterone, megestrol, melphalan, mercaptopurine, mesna, metformin, methotrexate, miltefosine, mitomycin, mitotane, mitoxantrone, MK-2206, mutamycin, N-(4-sulfamoylphenylcarbamothioyl) pivalamide, NF279, NF449, nilutamide, nocodazole, octreotide, olaparib, oxaliplatin, paclitaxel, pamidronate, pazopanib, pemexetred, pentostatin, perifosine, PF-04691502, plicamycin, pomalidomide, porfimer, PPADS, procarbazine, quercetin, raltitrexed, ramucirumab, reactive blue 2, rituximab, rolofylline, romidepsin, rucaparib, selumetinib, sirolimus, sodium 2,4-dinitrobenzenesulfonate, sorafenib, streptozocin, sunitinib, suramin, talazoparib, tamoxifen, temozolomide, temsirolimus, teniposide, testosterone, thalidomide, thioguanine, thiotepa, titanocene dichloride, tonapofylline, topotecan, trametinib, trastuzumab, tretinoin, veliparib, vinblastine, vincristine, vindesine, vinorelbine, and vorinostat (SAHA). In other embodiments, chemotherapeutic agents that may be conjointly administered with compounds provided herein include: ABT-263, dexamethasone, 5-fluorouracil, PF-04691502, romidepsin, and vorinostat (SAHA). In other embodiments, chemotherapeutic agents that may be conjointly administered with compounds provided herein include: 1-amino-4-phenylamino-9,10-dioxo-9,10-dihydroanthracene-2-sulfonate (acid blue 25), 1-amino-4-[4-hydroxyphenyl-amino]-9,10-dioxo-9,10-dihydroanthracene-2-sulfonate, 1-amino-4-[4-aminophenylamino]-9,10-dioxo-9,10-dihydroanthracene-2-sulfonate, 1-amino-4-[1-naphthylamino]-9,10-dioxo-9,10-dihydroanthracene-2-sulfonate, 1-amino-4-[4-fluoro-2-carboxyphenylamino]-9,10-dioxo-9,10-dihydroanthracene-2-sulfonate, 1-amino-4-[2-anthracenylamino]-9,10-dioxo-9,10-dihydroanthracene-2-sulfonate, APCP, β-methylene-ADP (AOPCP), capecitabine, cladribine, cytarabine, fludarabine, doxorubicin, gemcitabine, N-(4-sulfamoylphenylcarbamothioyl) pivalamide, NF279, NF449, PPADS, quercetin, reactive blue 2, rolofylline sodium 2,4-dinitrobenzenesulfonate, sumarin, and tonapofylline. In some embodiments, the chemotherapy agents are biologics such as ADCs or MET antibodies. In some embodiments, the chemotherapy agent is a MET inhibitor, a MEK inhibitor, a RET inhibitor, another ALK inhibitor, or ROS1 inhibitor. In some embodiments, the compound as described herein is used with one or more platininum based chemotherapy and/or immunotherapy (e.g. checkpoint inhibitors).

Many combination therapies have been developed for the treatment of cancer. In certain embodiments, compounds provided herein (e.g., compounds of Formula (I)) may be conjointly administered with one or more combination therapies. Examples of combination therapies with which compounds provided herein may be conjointly administered are included in Table 1.

TABLE 1
Exemplary combinatorial therapies for the treatment of cancer

| Name | Therapeutic agents |
|---|---|
| ABV | Doxorubicin, Bleomycin, Vinblastine |
| ABVD | Doxorubicin, Bleomycin, Vinblastine, Dacarbazine |
| AC (Breast) | Doxorubicin, Cyclophosphamide |
| AC (Sarcoma) | Doxorubicin, Cisplatin |
| AC (Neuroblastoma) | Cyclophosphamide, Doxorubicin |
| ACE | Cyclophosphamide, Doxorubicin, Etoposide |
| ACe | Cyclophosphamide, Doxorubicin |
| AD | Doxorubicin, Dacarbazine |
| AP | Doxorubicin, Cisplatin |
| ARAC-DNR | Cytarabine, Daunorubicin |
| B-CAVe | Bleomycin, Lomustine, Doxorubicin, Vinblastine |
| BCVPP | Carmustine, Cyclophosphamide, Vinblastine, Procarbazine, |
| | Prednisone |
| BEACOPP | Bleomycin, Etoposide, Doxorubicin, Cyclophosphamide, Vincristine, |
| | Procarbazine, Prednisone, Filgrastim |
| BEP | Bleomycin, Etoposide, Cisplatin |
| BIP | Bleomycin, Cisplatin, Ifosfamide, Mesna |
| BOMP | Bleomycin, Vincristine, Cisplatin, Mitomycin |
| CA | Cytarabine, Asparaginase |
| CABO | Cisplatin, Methotrexate, Bleomycin, Vincristine |
| CAF | Cyclophosphamide, Doxorubicin, Fluorouracil |
| CAL-G | Cyclophosphamide, Daunorubicin, Vincristine, Prednisone, |
| | Asparaginase |
| CAMP | Cyclophosphamide, Doxorubicin, Methotrexate, Procarbazine |
| CAP | Cyclophosphamide, Doxorubicin, Cisplatin |
| CAV | Cyclophosphamide, Doxorubicin, Vincristine |
| CAVE ADD | CAV and Etoposide |
| CA-VP16 | Cyclophosphamide, Doxorubicin, Etoposide |
| CC | Cyclophosphamide, Carboplatin |
| CDDP/VP-16 | Cisplatin, Etoposide |
| CEF | Cyclophosphamide, Epirubicin, Fluorouracil |
| CEPP(B) | Cyclophosphamide, Etoposide, Prednisone, with or without/ |
| | Bleomycin |
| CEV | Cyclophosphamide, Etoposide, Vincristine |
| CF | Cisplatin, Fluorouracil or Carboplatin Fluorouracil |
| CHAP | Cyclophosphamide or Cyclophosphamide, Altretamine, Doxorubicin, |
| | Cisplatin |
| ChlVPP | Chlorambucil, Vinblastine, Procarbazine, Prednisone |
| CHOP | Cyclophosphamide, Doxorubicin, Vincristine, Prednisone |
| CHOP-BLEO | Add Bleomycin to CHOP |
| CISCA | Cyclophosphamide, Doxorubicin, Cisplatin |
| CLD-BOMP | Bleomycin, Cisplatin, Vincristine, Mitomycin |
| CMF | Methotrexate, Fluorouracil, Cyclophosphamide |
| CMFP | Cyclophosphamide, Methotrexate, Fluorouracil, Prednisone |
| CMFVP | Cyclophosphamide, Methotrexate, Fluorouracil, Vincristine, |
| | Prednisone |
| CMV | Cisplatin, Methotrexate, Vinblastine |
| CNF | Cyclophosphamide, Mitoxantrone, Fluorouracil |
| CNOP | Cyclophosphamide, Mitoxantrone, Vincristine, Prednisone |
| COB | Cisplatin, Vincristine, Bleomycin |
| CODE | Cisplatin, Vincristine, Doxorubicin, Etoposide |
| COMLA | Cyclophosphamide, Vincristine, Methotrexate, Leucovorin, |
| | Cytarabine |
| COMP | Cyclophosphamide, Vincristine, Methotrexate, Prednisone |
| Cooper Regimen | Cyclophosphamide, Methotrexate, Fluorouracil, Vincristine, |
| | Prednisone |
| COP | Cyclophosphamide, Vincristine, Prednisone |
| COPE | Cyclophosphamide, Vincristine, Cisplatin, Etoposide |
| COPP | Cyclophosphamide, Vincristine, Procarbazine, Prednisone |
| CP (Chronic lymphocytic | Chlorambucil, Prednisone |
| leukemia) | |
| CP (Ovarian Cancer) | Cyclophosphamide, Cisplatin |
| CVD | Cisplatin, Vinblastine, Dacarbazine |
| CVI | Carboplatin, Etoposide, Ifosfamide, Mesna |
| CVP | Cyclophosphamide, Vincristine, Prednisome |
| CVPP | Lomustine, Procarbazine, Prednisone |
| CYVADIC | Cyclophosphamide, Vincristine, Doxorubicin, Dacarbazine |
| DA | Daunorubicin, Cytarabine |
| DAT | Daunorubicin, Cytarabine, Thioguanine |
| DAV | Daunorubicin, Cytarabine, Etoposide |
| DCT | Daunorubicin, Cytarabine, Thioguanine |
| DHAP | Cisplatin, Cytarabine, Dexamethasone |
| DI | Doxorubicin, Ifosfamide |
| DTIC/Tamoxifen | Dacarbazine, Tamoxifen |
| DVP | Daunorubicin, Vincristine, Prednisone |
| EAP | Etoposide, Doxorubicin, Cisplatin |
| EC | Etoposide, Carboplatin |
| EFP | Etoposie, Fluorouracil, Cisplatin |
| ELF | Etoposide, Leucovorin, Fluorouracil |
| EMA 86 | Mitoxantrone, Etoposide, Cytarabine |
| EP | Etoposide, Cisplatin |
| EVA | Etoposide, Vinblastine |
| FAC | Fluorouracil, Doxorubicin, Cyclophosphamide |
| FAM | Fluorouracil, Doxorubicin, Mitomycin |
| FAMTX | Methotrexate, Leucovorin, Doxorubicin |
| FAP | Fluorouracil, Doxorubicin, Cisplatin |
| F-CL | Fluorouracil, Leucovorin |
| FEC | Fluorouracil, Cyclophosphamide, Epirubicin |
| FED | Fluorouracil, Etoposide, Cisplatin |
| FL | Flutamide, Leuprolide |
| FZ | Flutamide, Goserelin acetate implant |
| HDMTX | Methotrexate, Leucovorin |
| Hexa-CAF | Altretamine, Cyclophosphamide, Methotrexate, Fluorouracil |
| IDMTX/6-MP | Methotrexate, Mercaptopurine, Leucovorin |
| IE | Ifosfamide, Etoposie, Mesna |
| IfoVP | Ifosfamide, Etoposide, Mesna |
| IPA | Ifosfamide, Cisplatin, Doxorubicin |
| M-2 | Vincristine, Carmustine, Cyclophosphamide, Prednisone, Melphalan |
| MAC-III | Methotrexate, Leucovorin, Dactinomycin, Cyclophosphamide |
| MACC | Methotrexate, Doxorubicin, Cyclophosphamide, Lomustine |
| MACOP-B | Methotrexate, Leucovorin, Doxorubicin, Cyclophosphamide, |
| | Vincristine, Bleomycin, Prednisone |
| MAID | Mesna, Doxorubicin, Ifosfamide, Dacarbazine |
| m-BACOD | Bleomycin, Doxorubicin, Cyclophosphamide, Vincristine, |
| | Dexamethasone, Methotrexate, Leucovorin |
| MBC | Methotrexate, Bleomycin, Cisplatin |
| MC | Mitoxantrone, Cytarabine |
| MF | Methotrexate, Fluorouracil, Leucovorin |
| MICE | Ifosfamide, Carboplatin, Etoposide, Mesna |
| MINE | Mesna, Ifosfamide, Mitoxantrone, Etoposide |
| mini-BEAM | Carmustine, Etoposide, Cytarabine, Melphalan |
| MOBP | Bleomycin, Vincristine, Cisplatin, Mitomycin |
| MOP | Mechlorethamine, Vincristine, Procarbazine |
| MOPP | Mechlorethamine, Vincristine, Procarbazine, Prednisone |
| MOPP/ABV | Mechlorethamine, Vincristine, Procarbazine, Prednisone, |
| | Doxorubicin, Bleomycin, Vinblastine |
| MP (multiple myeloma) | Melphalan, Prednisone |
| MP (prostate cancer) | Mitoxantrone, Prednisone |
| MTX/6-MO | Methotrexate, Mercaptopurine |
| MTX/6-MP/VP | Methotrexate, Mercaptopurine, Vincristine, Prednisone |
| MTX-CDDPAdr | Methotrexate, Leucovorin, Cisplatin, Doxorubicin |
| MV (breast cancer) | Mitomycin, Vinblastine |
| MV (acute myelocytic | Mitoxantrone, Etoposide |
| leukemia) | |
| M-VAC Methotrexate | Vinblastine, Doxorubicin, Cisplatin |
| MVP Mitomycin | Vinblastine, Cisplatin |
| MVPP | Mechlorethamine, Vinblastine, Procarbazine, Prednisone |
| NFL | Mitoxantrone, Fluorouracil, Leucovorin |
| NOVP | Mitoxantrone, Vinblastine, Vincristine |
| OPA | Vincristine, Prednisone, Doxorubicin |
| OPPA | Add Procarbazine to OPA. |
| PAC | Cisplatin, Doxorubicin |
| PAC-I | Cisplatin, Doxorubicin, Cyclophosphamide |
| PA-CI | Cisplatin, Doxorubicin |
| PCV | Lomustine, Procarbazine, Vincristine |
| PFL | Cisplatin, Fluorouracil, Leucovorin |
| POC | Prednisone, Vincristine, Lomustine |
| ProMACE | Prednisone, Methotrexate, Leucovorin, Doxorubicin, |
| | Cyclophosphamide, Etoposide |
| ProMACE/cytaBOM | Prednisone, Doxorubicin, Cyclophosphamide, Etoposide, Cytarabine, |
| | Bleomycin, Vincristine, Methotrexate, Leucovorin, Cotrimoxazole |
| PRoMACE/MOPP | Prednisone, Doxorubicin, Cyclophosphamide, Etoposide, |
| | Mechlorethamine, Vincristine, Procarbazine, Methotrexate, |
| | Leucovorin |
| Pt/VM | Cisplatin, Teniposide |
| PVA | Prednisone, Vincristine, Asparaginase |
| PVB | Cisplatin, Vinblastine, Bleomycin |
| PVDA | Prednisone, Vincristine, Daunorubicin, Asparaginase |
| SMF | Streptozocin, Mitomycin, Fluorouracil |
| TAD | Mechlorethamine, Doxorubicin, Vinblastine, Vincristine, Bleomycin, |
| | Etoposide, Prednisone |
| TTT | Methotrexate, Cytarabine, Hydrocortisone |
| Topo/CTX | Cyclophosphamide, Topotecan, Mesna |
| VAB-6 | Cyclophosphamide, Dactinomycin, Vinblastine, Cisplatin, |
| | Bleomycin |
| VAC | Vincristine, Dactinomycin, Cyclophosphamide |
| VACAdr | Vincristine, Cyclophosphamide, Doxorubicin, Dactinomycin, |
| | Vincristine |
| VAD | Vincristine, Doxorubicin, Dexamethasone |
| VATH | Vinblastine, Doxorubicin, Thiotepa, Flouxymesterone |
| VBAP | Vincristine, Carmustine, Doxorubicin, Prednisone |
| VBCMP | Vincristine, Carmustine, Melphalan, Cyclophosphamide, Prednisone |
| VC | Vinorelbine, Cisplatin |
| VCAP | Vincristine, Cyclophosphamide, Doxorubicin, Prednisone |
| VD | Vinorelbine, Doxorubicin |
| VelP | Vinblastine, Cisplatin, Ifosfamide, Mesna |
| VIP | Etoposide, Cisplatin, Ifosfamide, Mesna |
| VM | Mitomycin, Vinblastine |
| VMCP | Vincristine, Melphalan, Cyclophosphamide, Prednisone |
| VP | Etoposide, Cisplatin |
| V-TAD | Etoposide, Thioguanine, Daunorubicin, Cytarabine |
| 5 + 2 | Cytarabine, Daunorubicin, Mitoxantrone |
| 7 + 3 | Cytarabine with/, Daunorubicin or Idarubicin or Mitoxantrone |
| “8 in 1” | Methylprednisolone, Vincristine, Lomustine, Procarbazine, |
| | Hydroxyurea, Cisplatin, Cytarabine, Dacarbazine |

In certain embodiments, the conjoint therapies provided herein comprise conjoint administration with other types of chemotherapeutic agents, such as immuno-oncology agents. Cancer cells often have specific cell surface antigens that can be recognized by the immune system. Thus, immuno-oncology agents, such as monoclonal antibodies, can selectively bind to cancer cell antigens and effect cell death. Other immuno-oncology agents can suppress tumor-mediated inhibition of the native immune response or otherwise activate the immune response and thus facilitate recognition of the tumor by the immune system. Exemplary antibody immuno-oncology agents, include, but are not limited to, abagovomab, adecatumumab, afutuzumab, alemtuzumab, anatumomab mafenatox, apolizumab, blinatumomab, BMS-936559, catumaxomab, durvalumab, epacadostat, epratuzumab, indoximod, inotuzumab ozogamicin, intelumumab, ipilimumab, isatuximab, lambrolizumab, MED14736, MPDL3280A, nivolumab, obinutuzumab, ocaratuzumab, ofatumumab, olatatumab, pembrolizumab, pidilizumab, rituximab, ticilimumab, samalizumab, and tremelimumab. In some embodiments, the antibody immuno-oncology agents are selected from anti-CD73 monoclonal antibody (mAb), anti-CD39 mAb, anti-PD-1 mAb, and anti-CTLA4 mAb. Thus, in some embodiments, the methods provided herein comprise conjoint administration of one or more immuno-oncology agents, such as the agents mentioned above.

In some embodiments, the combination therapy comprises conjoint administration of a compound provided herein, such as a compound of Formula (I), with SH2 inhibitors, such as CGP78850, CPG85793, C90, C126, G7-18NATE, G7-B1, and NSC642056.

In some embodiments, the combination therapy comprises conjoint administration of a solid form or pharmaceutical composition provided herein, such as a compound of Formula (I), with ERK1/2 inhibitors such as ASN007, GDC-0994, KO-947, LTT462, LY3214996, MK-8353, ulixertinib.

In some embodiments, the combination therapy comprises conjoint administration of a compound provided herein, such as a compound of Formula (I), with MEK inhibitors, such as trametinib, cobimetinib, binimetinib, selumetinib, PD-325901, CI-1040, and TAK-733.

In some embodiments, the combination therapy comprises conjoint administration of a compound provided herein, such as a compound of Formula (I), with a MET inhibitor selected from JNJ-38877605, PF-04217903, foretinib, AMG 458, tivantinib, cabozantinib, crizotinib, capmatinib hydrochloride, tepotinib hydrochloride, and savolitinib.

In some embodiments, the combination therapy comprises conjoint administration of a compound of the disclosure, such as Formula (I), with a SHP2 inhibitor selected from TNO-155, RMC-4630, JAB-3068, or RLY-1971.

In some embodiments, the combination therapy comprises conjoint administration of a compound provided herein, such as a compound of Formula (I), with a RAS inhibitor selected from aliskiren, captopril, losartan, irbesartan, olmesartan, candesartan, valsartan, fimasartan, azilsartan, telmisartan, eprosartan, benazepril, enalapril, lisinopril, perindopril, quinapril, ramipril, and trandolapril.

In some embodiment, the combination therapy comprises administration of a compound provided herein, e.g., a compound of Formula (I), in combination with a TKI. In one embodiment, the TKI is a ROS1 inhibitor. In one embodiment, the TKI is an ALK inhibitor. In one embodiment, the TKI is crizotinib, ceritinib, alectinib, brigatinib, lorlatinib, entrectinib, repotrectinib, cabozantinib, foretinib, merestinib, taletrectinib, masitinib, or ensartinib. In one embodiment, the TKI is crizotinib. In one embodiment, the TKI is entrectinib. In one embodiment, the TKI is alectinib. In one embodiment, the TKI is brigatinib. In one embodiment, the TKI is lorlatinib.

In some embodiments, the combination therapy comprises conjoint administration of a compound provided herein, such as a compound of Formula (I), with anti-PD-1 therapy. In certain embodiments, the combination therapy comprises conjoint administration of a compound provided herein, such as a compound of Formula (I), with oxaliplatin. In other embodiments, the combination therapy comprises conjoint administration of a compound provided herein, such as a compound of Formula (I), with doxorubicin.

In certain embodiments, a compound provided herein may be conjointly administered with non-chemical methods of cancer treatment. In certain embodiments, a compound provided herein may be conjointly administered with radiation therapy. In certain embodiments, a compound provided herein may be conjointly administered with surgery, with thermoablation, with focused ultrasound therapy, with cryotherapy, or with any combination of these.

In certain embodiments, compounds provided herein may be conjointly administered with one or more other compounds provided herein. Moreover, such combinations may be conjointly administered with other therapeutic agents, such as other agents suitable for the treatment of cancer, immunological or neurological diseases, such as the agents identified above. In certain embodiments, conjointly administering one or more additional chemotherapeutic agents with a compound provided herein provides a synergistic effect. In certain embodiments, conjointly administering one or more additional chemotherapeutic agents provides an additive effect.

### Pharmaceutical Compositions

In certain embodiments, provided herein is a pharmaceutical preparation suitable for use in a human patient, comprising a compound provided herein (e.g., a compound of Formula (I), and one or more pharmaceutically acceptable excipients. In certain embodiments, the pharmaceutical preparations may be for use in treating or preventing a condition or disease as described herein. A compound provided herein may be used in the manufacture of medicaments for the treatment of any diseases or conditions disclosed herein.

In one embodiment, the pharmaceutical composition comprises a compound of Formula (I):

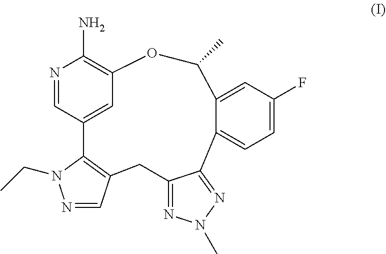 [SMILES: CCn1ncc2c1-c1cnc(N)c(c1)O[C@H](C)c1cc(F)ccc1-c1nn(C)nc1C2]

or a stereoisomer, or a mixture of stereoisomers thereof, or a pharmaceutically acceptable salt thereof, and a diluent, a binder, a disintegrant, and a lubricant. In one embodiment, Compound 1, or a stereoisomer, or a mixture of stereoisomers thereof, or a pharmaceutically acceptable salt thereof, in the pharmaceutical composition is free base of Compound 1. In one embodiment, the free base of Compound 1 is amorphous. In one embodiment, the free base of Compound 1 is one of the solid forms of free base of Compound 1 provided herein. In one embodiment, the free base of Compound 1 is Form 1 of the free base of Compound 1.

In one embodiment, the diluent is mannitol. In one embodiment, the diluent is Pearlitol 100SD, Pearlitol 110 C, Pearlitol 160 C, Pearlitol 25 C, Pearlitol 300 DC, Pearlitol 400 DC, Pearlitol 500 DC, Pearlitol SW—F 200, Parteck M 100, Parteck M 200, or lactose monohydrate. In one embodiment, the diluent is mannitol Pearlitol 50C. In one embodiment, the diluent is mannitol Pearlitol 200SD.

In one embodiment, the binder is hydroxypropyl cellulose (HPC), silicified microcrystalline cellulose (SMCC), or a mixture thereof. In one embodiment, the binder is HPC. In one embodiment, the binder is SMCC. In one embodiment, the binder is a mixture of HPC and SMCC. In certain embodiments, the binder is carboxymethylcellulose, alginates, gelatin, polyvinyl pyrrolidone, sucrose and/or acacia, or a mixture thereof.

In one embodiment, the disintegrant is sodium starch glycolate (SSG). In one embodiment, the disintegrant is cross-linked sodium carboxymethyl cellulose.

In one embodiment, the lubricant is magnesium stearate. In another embodiment, the lubricant is sodium lauryl sulfate.

In one embodiment, the diluent is mannitol, the binder is a mixture of HPC and SMCC, the disintegrant is sodium starch glycolate (SSG), and the lubricant is magnesium stearate.

In one embodiment, the tablets are coated with a non-functional film-coating (e.g., Opadry II Pink).

In one embodiment, the pharmaceutical composition provided herein is an oral dosage form. In one embodiment, the oral dosage form is a tablet. In one embodiment, the oral dosage form is an immediate release tablet.

The compositions and methods provided herein may be utilized to treat a subject in need thereof. In certain embodiments, the subject is a mammal such as a human, or a non-human mammal. When administered to subject, such as a human, the composition or the compound is preferably administered as a pharmaceutical composition comprising, for example, a compound provided herein and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In a preferred embodiment, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

A pharmaceutically acceptable carrier can contain physiologically acceptable agents that act, for example, to stabilize, increase solubility or to increase the absorption of a compound such as a compound provided herein. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound provided herein. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of a subject without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the subject. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. In certain embodiments, the unit dosage form is a tablet. In certain embodiments, the unit dosage form is a tablet of 5 mg (by weight of free base Compound 1) dose strength. In certain embodiments, the unit dosage form is a tablet of 25 mg (by weight of free base Compound 1) dose strength. The amount of active ingredient which can be combined with a carrier material to produce a single dosage form will vary depending upon the subject being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound provided herein, with the carrier and, optionally, one or more accessory ingredients. In general, the formulations are prepared by uniformly and intimately bringing into association a compound provided herein with liquid carriers, or finely divided solid carriers, or both, and then, if necessary, shaping the product.

Formulations provided herein suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound provided herein as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the pharmaceutical compositions for administration to the mouth may be presented as a mouthwash, or an oral spray, or an oral ointment.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administration also include pessaries, tampons, creams, gels, pastes, foams or spray formulations containing such carriers as are known in the art to be appropriate.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound provided herein to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this disclosure. Exemplary ophthalmic formulations are described in U.S. Publication Nos. 2005/0080056, 2005/0059744, 2005/0031697 and 2005/004074 and U.S. Pat. No. 6,583,124, the contents of which are incorporated herein by reference. If desired, liquid ophthalmic formulations have properties similar to that of lacrimal fluids, aqueous humor or vitreous humor or are compatible with such fluids. A preferred route of administration is local administration (e.g., topical administration, such as eye drops, or administration via an implant).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions provided herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods provided herein, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinacious biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the subject being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the subject's condition, the disorder being treated, the stability of the compound, and, if desired, another type of therapeutic agent being administered with the compound provided herein. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

If desired, the effective daily dose of the active compound may be administered as one, two, three, four, five, six or more sub-doses administered separately at appropriate intervals throughout the day, optionally, in unit dosage forms. In certain embodiments provided herein, the active compound may be administered two or three times daily. In preferred embodiments, the active compound will be administered once daily.

In certain embodiments, compounds provided herein may be used alone or conjointly administered with another type of therapeutic agent. As used herein, the phrase “conjoint administration” refers to any form of administration of two or more different therapeutic compounds such that the second compound is administered while the previously administered therapeutic compound is still effective in the body (e.g., the two compounds are simultaneously effective in the subject, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, a subject who receives such treatment can benefit from a combined effect of different therapeutic compounds.

In certain embodiments, conjoint administration of compounds provided herein with one or more additional therapeutic agent(s) (e.g., one or more additional chemotherapeutic agent(s)) provides improved efficacy relative to each individual administration of the compound provided herein or the one or more additional therapeutic agent(s). In certain such embodiments, the conjoint administration provides an additive effect, wherein an additive effect refers to the sum of each of the effects of individual administration of the compound provided herein and the one or more additional therapeutic agent(s).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

The disclosure now being generally described, it will be more readily understood by reference to the following examples which are included merely for purposes of illustration of certain aspects and embodiments of the present disclosure, and are not intended to limit the disclosure.

### Examples

### Example 1

Generation of Ba/F3 Stable Cell Lines. Genes encoding CD74-ROS1 wild-type (wt) and CD74-ROS1 G2032R were synthesized (GeneRay), cloned into the retroviral construct pMSCV-puro (Biovector), and packaged into retroviral particles. The virus was used to infect Ba/F3 cells (RIKEN) at multiplicity of infection=1 for 1 day. Infected cells were rescued in media (RPMI-1640 with 10% fetal bovine serum and 1% streptomycin and penicillin) supplemented with mouse IL-3 (10 ng/mL) for 2 days, and stable cell lines were selected by IL-3 withdrawal and puromycin (0.8 μg/mL) for 7 days. Monoclones were selected by single-cell dilution in IL-3-free medium containing puromycin (0.8 μg/mL). Transformation of desired genes was confirmed by Sanger sequencing and western blot using ROS1 antibody (CST #3287)

Cell Proliferation Assay. Stable cells were plated at 1,000 cells/well (40 μL) in a 384-well plate for 1 day. Test compounds (40 nL) were then added in a 3-fold dilution series using the TECAN EVO200 liquid handler and incubated for 72 hours. Plates were equilibrated at room temperature for 15 minutes followed by addition of 40 μL CellTiter-Glo reagent (Promega). Luminescence was measured on a plate reader. Half-maximal inhibitory concentration (IC₅₀) was calculated from percent inhibition and inhibitor concentration using four-parameter logistic regression.

The results of Compound 1 and several ROS1 inhibitors are shown below.

| Model | | | | | | | | | | | |
|---|---|---|---|---|---|---|---|---|---|---|---|
| name | ROS1 Status | Compound 1 | | Crizotinib | | Entrectinib | | Lorlatinib | | Repotrectinib | |
| Ba/F3 | CD74-ROS1 | 1.2 | nM | 40 | nM | 23 | nM | 1.3 | nM | 4.4 | nM |
| Ba/F3 | CD74-ROS1 G2032R | 3.5 | nM | 960 | nM | 1,500 | nM | 300 | nM | 25 | nM |
| Ba/F3 | CD74-ROS1 S1986F | <0.58 | nM | 39 | nM | 26 | nM | <0.27 | nM | 0.84 | nM |
| Ba/F3 | CD74-ROS1 L2026M | 1.5 | nM | 110 | nM | 41 | nM | 0.77 | nM | 3.3 | nM |
| Ba/F3 | CD74-ROS1 D2033N | 1.0 | nM | 77 | nM | 79 | nM | 0.44 | nM | 2.5 | nM |

### Example 2. In vivo evaluation of Compound 1

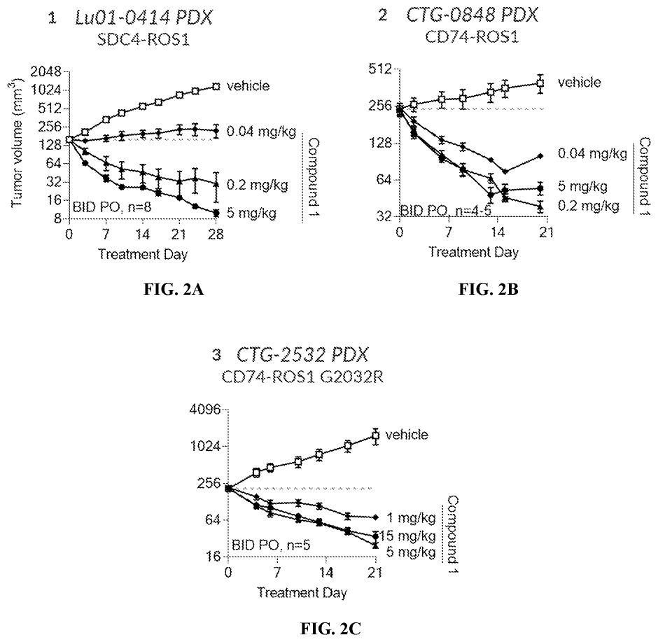

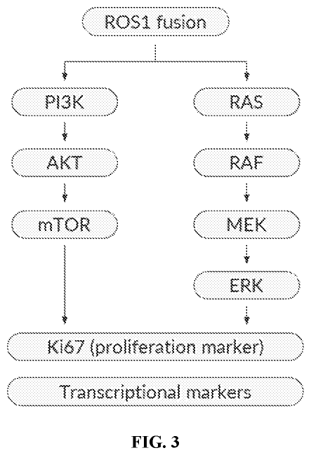

Model 2A: Female Balb/c nude mice were implanted subcutaneously into the right flank with tumor slices (˜30 mm³) from model LU-01-0414, which was derived from a 51-year-old female with NSCLC and unknown smoking history. The tumor was a G3, and the tumor stage was T2N2M0. The tumor harbored SDC4-ROS1. In the efficacy study, after tumors grew to an average tumor volume of 160 mm³, mice (n=5-8/group) were randomized and administered by oral gavage twice daily (BID; 12-hour intervals) vehicle or Compound 1 for 28 days. The results are shown in FIG. 2A.

Model 2B: Female athymic Nude-Foxn1^{nu} mice were implanted subcutaneously into the left flank with tumor fragments from model CTG-0848, which was derived from a 41-year-old Asian male with Stage II NSCLC. The tumor harbored CD74-ROS1 fusion. After tumors grew to approximately 240 mm³, mice (n=5-5/group) were randomized and administered vehicle or Compound 1 by oral gavage twice-daily (BID, 12 hour intervals) for 21 days of dosing (BID×21 days). The results are shown in FIG. 2B.

Model 2C: Female athymic Nude-Foxn1^{nu} mice were implanted subcutaneously into the left flank with tumor fragments from model CTG-2532, which was derived from a female Asian non-smoker with stage IV NSCLC whose disease responded to the TKI crizotinib and then progressed. The tumor harbored CD74-ROS1 G2032R. After tumors grew to approximately 215, mice (n=5/group) were randomized and administered vehicle or 1, 5, or 15 mg/kg of Compound 1 by oral gavage twice-daily (BID, 12 hour intervals) for 21 days of dosing (BID×21 days). The results are shown in FIG. 2C.

### Example 3. In vivo evaluation of Compound 1

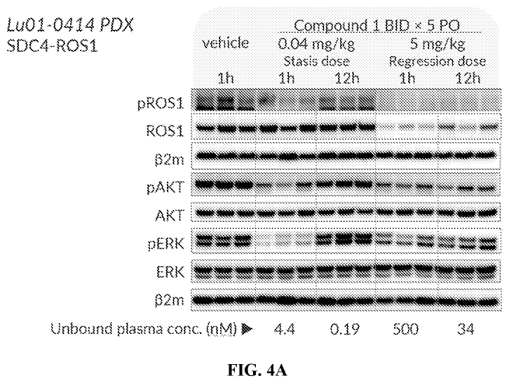

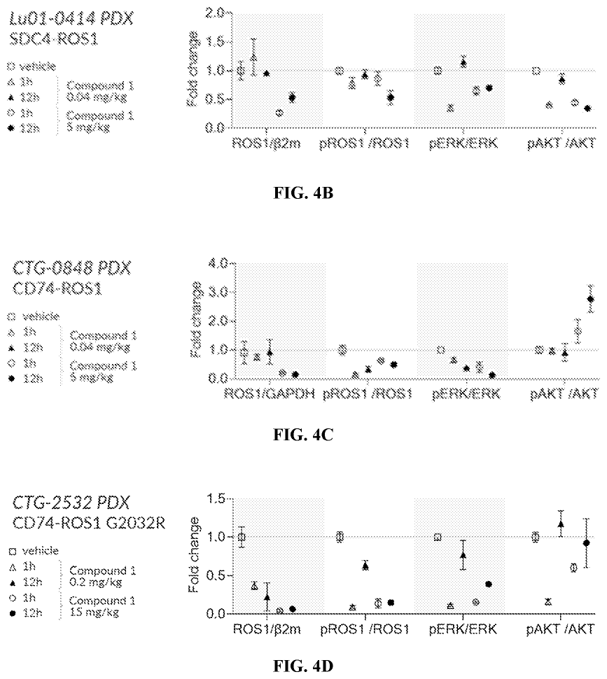

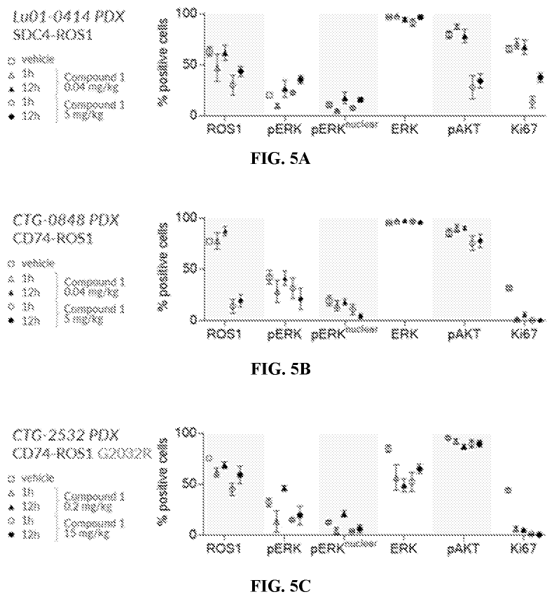

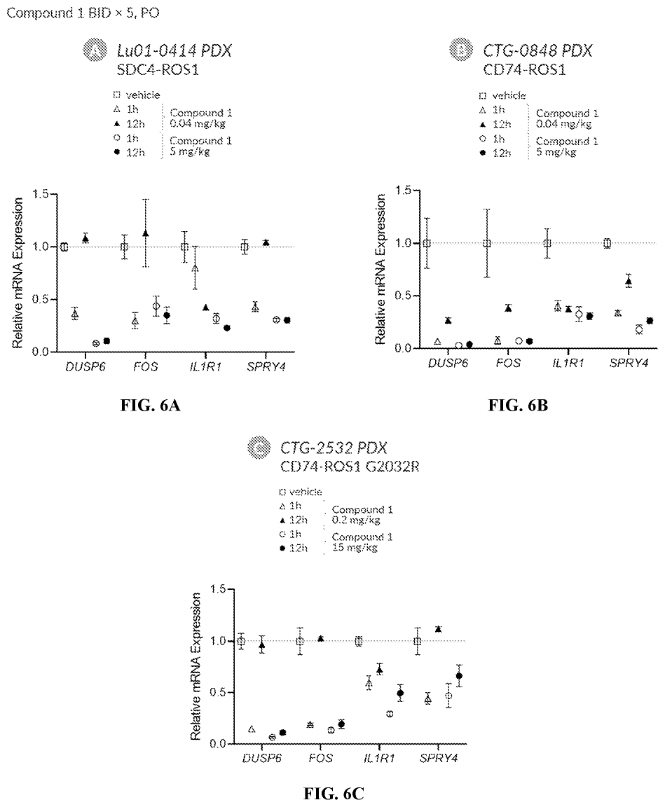

Model 1 (LU-01-0414 PDX SDC4-ROS1): Female Balb/c nude mice were implanted subcutaneously into the right flank with tumor slices (˜30 mm³) from model LU-01-0414, which was derived from a 51-year-old female with NSCLC and unknown smoking history. The tumor was a G3, and the tumor stage was T2N2M0. The tumor harbored SDC4-ROS1. After tumors grew to an average tumor volume of 492 mm³, mice received a single dose or BID×5 days of vehicle or Compound 1, and tumor and blood were collected at 1 hour and 12 hours (treatment only) post last dose. Tumors were divided and snap frozen and processed for western blot (FIG. 4A and FIG. 4B, described below) or fixed in formalin for 24-48 hours followed by FFPE block preparation and processed for immunohistochemical analysis (FIG. 5A, described below) or gene expression analysis by NanoString assay (FIG. 6A, described below). Blood samples were collected into tubes containing EDTA-K2, centrifuged, and the resulting plasma was analyzed by LC/MS/MS to determine the total concentration of Compound 1 in the plasma. The free concentration of Compound 1 (FIG. 4A) was calculated by multiplying the unbound fraction for Compound 1 in female Balb/c mouse plasma determined by equilibrium dialysis.

Model 2 (CTG-0848 PDX CD74-ROS1): Female athymic Nude-Foxn1^{nu} mice were implanted subcutaneously into the left flank with tumor fragments from model CTG-0848, which was derived from a 41-year-old Asian male with Stage II NSCLC. The tumor harbored CD74-ROS1 fusion. After tumors grew to 350 to 500 mm³, mice received a single dose or BID×5 days of vehicle or Compound 1, and tumors were collected at 1 hour and 12 hours (treatment only) post last dose and processed as described for Model 1 for western blot analysis (FIG. 4C), immunohistochemical analysis (FIG. 5B), or gene expression analysis (FIG. 6B).

Model 3 (CTG-2532 PDX CD74-ROS1 G2032R): Female athymic Nude-Foxn1^{nu} mice were implanted subcutaneously into the left flank with tumor fragments from model CTG-2532, which was derived from a female Asian non-smoker with stage IV NSCLC whose disease responded to the TKI crizotinib and then progressed. The tumor harbored CD74-ROS1 G2032R. After tumors grew to 350 to 500 mm³, mice received a single or BID×5 days (11 total doses) of vehicle or Compound 1, and tumors were collected at 1 hour and 12 hours (treatment only) post last dose and processed as described for Model 1 for western blot analysis (FIG. 4D), immunohistochemical analysis (FIG. 5C), or gene expression analysis (FIG. 6C).

The following preparation has been used in these three models to evaluate the inhibitory activity of Compound 1 in ROS1 signaling in tumors.

For western blot analysis, snap-frozen tumors were cut, lysed with RIPA buffer (containing protease and phosphatase inihibitor cocktails), grinded, and centrifuged. Supernatants were collected, proteins quantified, and similar concentrations of supernatant samples were separated by electrophoresis and transferred to nitrocellulose membranes. Membranes were cut, blotted, treated with HRP substrate and chemiluminescence was detected by Amersham Imager 680. Intensity of individual bands were quantified using 1D quant (GE) densitometry software. The following antibodies were used: phospho-ROS1 (Tyr2274), Cell Signaling Technology (CST) 3078; ROS1 (D4D6) CST 3287; phospho-AKT (Ser473) (D9E), CST 4060; AKT, CST 9272; phospho-p44/42 MAPK (Erk1/2) (Thr202/Tyr204), CST 9101; p44/42 MAPK (Erk1/2), CST 9102; B2-microglobulin (β2m), CST 12851; GAPDH (D4C6R), CST 97166; goat anti-rabbit IgG H&L (HRP), Abcam ab6721; and goat anti-mouse IgG H&L (HRP), Abcam ab97023.

For immunohistochemical analysis, FFPE blocks were sectioned, stained automatically with the Leica Bond RX system, and imaged with the Leica Aperio Versa8. Tumor regions were manually selected in HALO (Indica Labs) and percentage of positive cells were automatically recorded. The following antibodies were used: ROS1, CST 63452; Rabbit IgG, Abcam 172730; ERK, CST 4695; phospho-ERK, CST 4376; and Ki-67, CST 9027.

For gene expression analysis by NanoString assay, RNA was extracted from FFPE sections by RNAstorm FFPE RNA Extraction Kit, quantified by NanoDrop, Qubit and Agilent BioAnalyzer 2100, and analyzed by NanoString nCounter PanCancer Human Pathways Panel according to manufacturer instructions. NanoString data were analyzed by Excel and packages from R/Biocondutor and included quality control of the experiment, background correlation and linearity by negative and positive spike-ins, normalization based on housekeeping genes by Median of Ratio method, and differential gene expression analysis by Walt Test method with p-values corrected for multiple testing with Benjamini-Hochberg method.

### Example 4: Phase 1/2 Clinical Study

A FIH, Phase 1/2, multicenter, open-label, dose escalation and expansion study is conducted to evaluate the safety and tolerability of Compound 1, to determine the RP2D, and to evaluate the antitumor activity in patients with advanced ROS1-positive NSCLC and other advanced ROS1-positive solid tumors.

### Phase 1 Objectives:

Primary Objective: to determine the RP2D and/or maximum tolerated dose (MTD) of Compound 1 in patients with advanced ROS1-positive solid tumors.

Secondary Objectives: to evaluate the overall safety and tolerability of Compound 1; to characterize the PK profile of Compound 1; to evaluate preliminary antitumor activity of Compound 1 in patients with advanced ROS1 positive solid tumors.

### Phase 2 Objectives:

Primary Objective: to evaluate the ORR of Compound 1 at the RP2D in patients with advanced ROS1-positive NSCLC and other solid tumors.

Secondary Objectives: to assess additional measures of clinical efficacy in patients with ROS1-positive NSCLC and other solid tumors; to evaluate the intracranial antitumor activity of Compound 1 at the RP2D in patients with advanced ROS1-positive NSCLC and other solid tumors; to characterize the safety and tolerability of Compound 1 at the RP2D; to confirm the PK profile of Compound 1 at the RP2D.

### Phase 1 Endpoints:

Primary Endpoint: Incidence of dose-limiting toxicities (DLTs) during Cycle 1

Secondary Endpoints:
Incidence and severity of treatment-emergent adverse events (TEAEs) and changes in clinically relevant laboratory parameters
Pharmacokinetic parameters of Compound 1: Maximum plasma concentration (Cₘₐₓ); Cₘₐₓ-dose normalized, plasma concentration at the end of the dosing interval (Cₜₐᵤ); time of maximum concentration (Tₘₐₓ); area under the curve at the end of the dosing interval (AUCₜₐᵤ); AUCₜₐᵤ-dose normalized, area under the curve from time 0 to 24 (AUC₀₋₂₄); AUC₀₋₂₄-dose normalized, area under the curve from time 0 to infinity (AUC_{inf}); AUC_{inf}-dose normalized; oral clearance (CL/F); volume of distribution (V_{z}/F); and half-life (t_{1/2})
Objective response rate (ORR) per Response Evaluation Criteria in Solid Tumors Version 1.1 (RECIST 1.1)-Defined as the percent of patients with a complete response (CR) or partial response (PR) according to RECIST 1.1
Duration of response (DOR) per RECIST 1.1—In responders, defined as the time from first response per RECIST 1.1 to radiographic disease progression or death
Intracranial ORR (IC-ORR) per RECIST 1.1—In patients with measurable metastatic CNS disease up to 5 intracranial target lesions are accessed for response, according to RECIST 1.1
Intracranial DOR (IC-DOR) per RECIST 1.1—In responders with metastatic CNS disease, defined as the time from first IC-response per RECIST 1.1 to radiographic IC-disease progression or death
Clinical benefit rate (CBR) per RECIST 1.1-Defined as the percent of patients with a confirmed CR or PR, or stable disease (SD) of at least 24 weeks duration according to RECIST 1.1
Time to response per RECIST 1.1-Defined as the time from first dose to first confirmed radiographic response according to RECIST 1.1
Progression-free survival (PFS) per RECIST 1.1-Defined as the time from first dose to radiographic disease progression per RECIST 1.1 or death

Exploratory Endpoints:
Pharmacodynamic markers associated with response to Compound 1
Extracranial ORR (EC-ORR) per RECIST 1.1
In patients with extracranial metastatic disease, defined as the percent of patients with an extracranial response according to RECIST 1.1 per Investigator assessment
Evaluate metabolite profile of Compound 1

### Phase 2 Endpoints:

Primary Endpoint: ORR per RECIST 1.1-Defined as the proportion of patients with a confirmed CR or PR according to RECIST 1.1 per Blinded Independent Central Review (BICR) of the patients confirmed to have measurable disease at baseline per BICR.

Secondary Endpoints
DOR per RECIST 1.1—In responders, defined as the time from first BICR-assessed confirmed response per RECIST 1.1 to radiographic disease progression or death
IC-ORR per RECIST 1.1—In patients with measurable metastatic CNS disease up to 5 intracranial target lesions are assessed for response according to RECIST 1.1 per BICR of the patients confirmed to have measurable intracranial disease at baseline per BICR
IC-DOR per RECIST 1.1—In responders with metastatic CNS disease, defined as the time from first BICR-assessed confirmed response per RECIST 1.1 to radiographic disease progression or death.
CBR per RECIST 1.1-Defined as the percent of patients with a confirmed CR or PR, or SD of at least 24 weeks duration according to RECIST 1.1 per BICR
Time to response per RECIST 1.1-Defined as the time from first dose to first confirmed radiographic response according to RECIST 1.1 per BICR
Time to IC-response per RECIST 1.1—In patients with metastatic CNS disease, defined as the time from first dose to first confirmed radiographic IC-response according to RECIST 1.1 per BICR
Incidence and severity of TEAEs and changes in clinically relevant laboratory parameters
Pharmacokinetic parameters of Compound 1-Cₘₐₓ, Cₘₐₓ-dose normalized, Cₜₐᵤ, Tₘₐₓ, AUCₜₐᵤ, AUCₜₐᵤ-dose normalized, AUC₀₋₂₄, AUC₀₋₂₄-dose normalized, AUC_{inf}, AUC_{inf}
dose normalized, CL/F, V_{z}/F, t_{1/2}
PFS per RECIST 1.1-Defined as the time from first dose to radiographic disease progression per RECIST 1.1 based on BICR assessment or death
Intracranial PFS (IC-PFS) per RECIST 1.1—In patients with metastatic CNS disease, defined as the time from first dose to radiographic disease progression per RECIST 1.1 based on BICR assessment or death
Overall survival (OS)-Defined as the time from first dose to death due to any cause

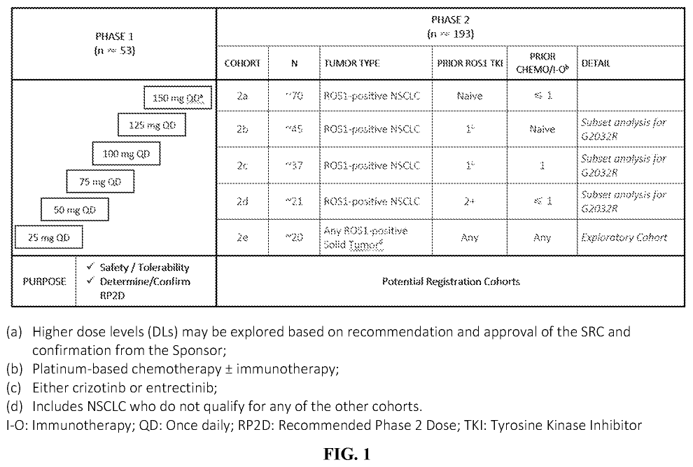

Study Design: The study is conducted in 2 phases (FIG. 1).
Phase 1 employs a Bayesian Optimal Interval Design (BOIN) with 3+3 Run-in Dose Escalation: Patients with advanced/metastatic ROS1-positive solid tumors treated with at least 1 prior ROS1 TKI therapy.
Phase 2 will include 5 cohorts:
   Cohort 2a: Patients with advanced/metastatic ROS1-positive NSCLC naïve to TKI therapy. Up to one prior platinum-based chemotherapy with or without immunotherapy is allowed.
   Cohort 2b: Patients with advanced/metastatic ROS1-positive NSCLC treated with 1 prior ROS1 TKI (either crizotinib or entrectinib) and no prior platinum-based chemotherapy or immunotherapy allowed.
   Cohort 2c: Patients with advanced/metastatic ROS1-positive NSCLC treated with 1 prior ROS1 TKI (either crizotinib or entrectinib) and 1 prior platinum-based chemotherapy with or without immunotherapy.
   Cohort 2d: Patients with advanced/metastatic ROS1-positive NSCLC treated with ≥2 prior ROS1 TKIs and up to 1 prior platinum-based chemotherapy with or without immunotherapy.
   Cohort 2e (exploratory): Patients with any advanced/metastatic ROS1-positive solid tumor (including patients with ROS1-positive NSCLC not otherwise eligible for any other cohorts) and progressed on any prior therapy (includes, but is not limited to, patients who have progressed on prior ROS1 TKIs).

The Phase 1 portion of the study employ a BOIN design with 3+3 run-in to evaluate dose escalation. The primary objective of the Phase 1 portion of the study is to determine the RP2D and to assess the safety of Compound 1 in patients with solid tumors. The safety profile is assessed based on physical examinations; ECOG PS; changes from baseline in laboratory parameters, ECGs, vital signs, and ophthalmic examination (baseline only and as clinically indicated); and the reporting of AEs. The DLT Observation Period is defined from first dose on day 1 of cycle 1 (C1D1) through to the end of the first cycle (28-day cycle period).

After patients are treated at a given dose level and monitored for 28 days, all available safety data are reviewed. Initiation of the next dosing group depends on the outcome of the BOIN analysis.

The selection of the RP2D occurs after a sufficient number of patients have enrolled and completed the first imaging/efficacy assessment and the data have been reviewed.

Phase 1 Dose Levels: Phase 1 evaluates ascending doses of Compound 1 monotherapy administered orally once daily (QD) continuously until disease progression, unacceptable toxicity, or voluntary withdrawal of consent. Each cycle of treatment is 28 days. Under the BOIN design, up to approximately 53 patients are enrolled and treated at the dose levels presented in the following table.

| Dose level | Compound 1 Dose | | Frequency* |
|---|---|---|---|
| DL1 | 25 | mg | QD |
| DL2 | 50 | mg | QD |
| DL3 | 75 | mg | QD |
| DL4 | 100 | mg | QD |
| DL5 | 125 | mg | QD |
| DL6** | 150 | mg | QD |

*BID dosing may be explored at any DL.
**Higher DLs may be explored.

Intermediate dose levels may be explored during dose escalation; lower dose levels (e.g., 10 mg QD and 5 mg QD) may be explored as additional dose levels based on results of the BOIN.

Recommended Phase 2 Dose: The recommended Phase 2 dose (RP2D) is expected to be an MTD or a lower optimal biological dose, or a maximum feasible dose, and is the dose level deemed as suitable for testing in the Phase 2 portion of the study. The RP2D is anticipated to achieve sufficient target coverage of the G2032R ROS1 mutation in the periphery and CNS, based on PK modeling simulations; the RP2D also demonstrates a tolerable safety profile along with preliminary antitumor activity. The selection of the RP2D is made using the totality of the data obtained from Phase 1, and includes the evaluation of clinical PK, pharmacodynamics, safety, and antitumor activity.

Duration of Treatment: Across both phases, patients receive study drug continuously from first dose until independent radiological confirmation of disease progression, unacceptable toxicity, withdrawal by patient, termination the study, or commercial availability. Patients may continue to receive Compound 1 following progression in the body or brain suitable for local ablation.

End of Study Definition: The end of the study is defined as the date of the last visit of the last patient in the study or termination of the study.

Inclusion Criteria: Patients must meet all of the following criteria to be eligible to enroll in the study:

1. Age≥18 years. (Phase 2 Cohort 2e only: Age≥12 years and weighing ≥40 kg.)
2. Disease criteria
a. Phase 1: Histologically or cytologically confirmed metastatic solid tumor with documented ROS1 rearrangement determined by testing in a Clinical Laboratory Improvement Amendments (CLIA) laboratory in the US or equivalently accredited diagnostic lab outside the United States (US) and using a local diagnostic test or a commercial test or by a regulatory agency approved fluorescence in situ hybridization (FISH) or next generation sequencing (NGS) or reverse transcription polymerase chain reaction (RT-PCR).
b. Cohorts 2a, 2b, 2c and 2d: Histologically or cytologically confirmed metastatic NSCLC with ROS1 rearrangement as determined by testing in a CLIA or equivalently accredited diagnostic lab using a local diagnostic test or a commercial test or by a regulatory agency approved FISH or NGS or RT-PCR.
c. Cohort 2e: Histologically or cytologically confirmed metastatic solid tumor (other than NSCLC) with ROS1 rearrangement as determined by testing in a CLIA or equivalently accredited diagnostic lab using a local diagnostic test or a commercial test or by a regulatory agency approved FISH or NGS or RT-PCR.
   3. Prior anticancer treatment
a. Phase 1: Must have received at least 1 prior ROS1 TKI therapy; any number of prior platinum-based chemotherapies with or without immunotherapy is allowed.
b. Cohort 2a: Must be naïve to TKI therapy and up to one prior platinum-based chemotherapy (with or without immunotherapy).
c. Cohort 2b: Must have received 1 prior ROS1 TKI therapy (either crizotinib or entrectinib) and no prior platinum-based chemotherapy or immunotherapy.
d. Cohort 2c: Must have received 1 prior ROS1 TKI therapy (either crizotinib or entrectinib) and 1 prior platinum-based chemotherapy (with or without immunotherapy)
e. Cohort 2d: Must have received at least 2 prior ROS1 TKI therapies and up to 1 prior platinum-based chemotherapy (with or without immunotherapy).
f. Cohort 2e: Must have progressed on any prior therapy (includes, but is not limited to, patients who have progressed on prior ROS1 TKIs).
   4. Phase 1: Must have evaluable disease (target or nontarget) according to RECIST 1.1. Phase 2: Must have measurable disease, defined as ≥1 radiologically measurable target lesion according to RECIST 1.1 (Oken et al., Am. J. Clin. Oncol. 1982, 5 (6): 649-655). In all study parts, patients with asymptomatic CNS-only measurable disease ≥10 mm as defined by RECIST 1.1 are eligible.
   5. Patient agrees to provide tumor tissue (archived, if available, or a fresh biopsy) for ROS1 status confirmation and is willing to consider an on-treatment tumor biopsy, if considered safe and medically feasible.
   6. Eastern Cooperative Oncology Group (ECOG) performance status (PS) of 0, 1 or 2 (Eisenhauer et al., Eur. J. Cancer 2009, 45 (2): 228-247).
   7. Adequate organ function and bone marrow reserve as indicated by the following laboratory assessments performed prior to the first dose of study drug:
a. Bone marrow function: absolute neutrophil count (ANC) ≥1500/μL; platelet count >75,000/μL; hemoglobin >8 g/dL.
b. Renal function: estimated creatinine clearance ≥60 mL/min per Cockroft-Gault formula.
c. Hepatic function: bilirubin <1.5×ULN, unless evidence of Gilbert Syndrome, in which the patient must have total bilirubin <3.0 mg/dL; aspartate aminotransferase and alanine aminotransferase ≤3.0×ULN (≤5.0×ULN if liver metastases involvement).
   8. All clinically relevant toxicities related to prior anticancer therapy must have recovered to Grade ≤1 or baseline (except alopecia or ototoxicity).
   9. Women of childbearing potential (WOCBP) and male patients must be willing to abstain from sexual activity or use an effective contraceptive method or be surgically sterile from the time of signing the informed consent form (ICF) through the duration of the study and for 30 days for women and 90 days for men after the last administration of study drug. Effective contraception for WOCBP includes 1 “highly effective method” or 2 “effective” methods based on World Health Organization (WHO) criteria.
   10. Provide written informed consent and willing and able to comply with requirements of the study protocol. Assent must be obtained for patients <18 years old (Phase 2 Cohort 2e only) and a parent/guardian must provide written consent.

Exclusion Criteria: Patients meeting any of the following criteria are excluded from the study:

1. Patient's cancer has a known primary driver alteration other than ROS1. For example, NSCLC with a targetable mutation in EGFR, ALK, MET, RET, or BRAF; colorectal with an oncogenic KRAS, NRAS, or BRAF mutation.
2. Known allergy/hypersensitivity to excipients of Compound 1.
3. Major surgery within 4 weeks of study entry. Minor surgical procedures (e.g., port insertion) are permitted, but with sufficient time for wound healing as deemed clinically appropriate.
4. Ongoing or recent anticancer therapy within the following timeframe prior to first dose of study drug (Compound 1 may be started within limits for prior TKI or chemotherapy if considered to be safe and within the best interest of the patient, with prior approval):
a. TKI or other non-chemotherapy/non-immunotherapy anticancer agents <5 half-lives or <7 days, whichever is longer
b. Chemotherapy <21 days
c. Immunotherapy or cellular therapy <28 days
   5. Ongoing or recent radiation therapy within the following timeframe prior to first dose of study drug:
a. Radiation therapy (except palliative radiation to relieve bone pain)<14 days
b. Palliative radiation to relieve bone pain <48 hours
c. Stereotactic or small field brain irradiation <7 days
d. Whole brain radiation <14 days
   6. Prior high-dose chemotherapy requiring stem cell rescue.
   7. Uncontrolled clinically relevant bacterial or fungal infection requiring systemic therapy.
   8. Has known active tuberculosis or active Hepatitis B or C. Active Hepatitis B is defined as a known positive HBsAg result. Active Hepatitis C is defined by a known positive Hep C Ab result and known quantitative HCV RNA results greater than the lower limits of detection of the assay.
   9. Patient has a QTcF consistently >450 msec. Patient has a history of prolonged QT syndrome or Torsades de pointes.
   10. Patients with clinically significant cardiovascular disease as follows:
a. Within 3 months of enrollment: cerebral vascular accident/stroke; myocardial infarction; unstable angina; uncontrolled atrial fibrillation of any grade
b. History of congestive heart failure (New York Heart Association Classification Class ≥II); second-degree or third-degree atrioventricular block (unless paced) or any atrioventricular block with PR consistently ≥220 msec; or ongoing cardiac dysrhythmias of NCI-CTCAE Grade ≥2.
   11. Patient has central nervous system (CNS) metastases or a primary CNS tumor that is associated with progressive neurological symptoms or requires increasing doses of corticosteroids to control the CNS disease. If a patient requires corticosteroids for management of CNS disease, the dose must have been stable for the 2 weeks preceding C1D1.
   12. Symptomatic spinal cord compression.
   13. Patients with moderate to severe cognitive impairment or psychiatric disturbances that would compromise the patient's ability to comply with study requirements.
   14. Evidence of active malignancy (other than current ROS1-positive solid malignancy) requiring systemic therapy within the prior 2 years.
a. Exceptions: nonmelanoma skin cancer, in situ melanoma, in situ cervical cancer, papillary thyroid cancer, ductal carcinoma in situ of the breast, or localized and presumed cured prostate cancer. Patients on long-term anti-hormonal therapy for a prior malignancy are allowed as long as the malignancy has not been active within the prior 2 years.
   15. Concomitant use (within 12 days of enrollment) of strong CYP3A4 inducers or strong CYP3A4 inhibitors.
   16. Manifestation of malabsorption due to prior gastrointestinal surgery, disease, or other illness that could affect oral absorption, distribution, metabolism, or excretion of the study drug.
   17. Patient is pregnant or breast-feeding. WOCBP must have a negative serum pregnancy test at Screening and urine tests prior to first dose of study drug.
   18. Actively receiving systemic treatment or direct medical intervention on another therapeutic clinical study.
   19. Any medical condition or laboratory abnormality that would pose a risk to study patient or confound the ability to interpret study results.

Study Drug, Dosage, and Route of Administration: Compound 1 is supplied as tablets for oral administration in 2 strengths: 5 mg and 25 mg provided in 32-count high-density polyethylene bottles with an induction seal/child resistant cap oral administration. Compound 1 should be taken on an empty stomach, at least 1 hour before and no sooner than 2 hours after ingestion of food and/or beverages other than water.

### Prior and Concomitant Medications and Therapies:

Compound 1 was not considered a substrate for CYP2B6, CYP2C9, CYP2C19 or CYP2D6. Compound 1 is a substrate of CYP3A4 with the potential for contributions from CYP1A2 and CYP2C8.

Compound 1 was not considered an inhibitor of CYP1A2, CYP2B6 or CYP2D6 (IC₅₀>100 μM) and was a weak inhibitor of CYP2C8 (IC₅₀=70 μM), CYP2C9 (IC₅₀=70.3 μM), CYP3A4 (testosterone IC₅₀=62.7 μM), CYP2C19 (IC₅₀=38.4 μM) and CYP3A4 (midazolam IC₅₀=30 μM). Compound 1 was a potential inhibitor of CYP3A4 (testosterone) and CYP3A4 (midazolam). Compound 1 was a potential inducer of CYP3A4.

In certain embodiments, concomitant use (within 12 days of enrollment) of drugs that are known strong CYP3A4 inducers (such as phenobarbital, rifampin, phenytoin, carbamazepine, rifabutin, rifapentine, clevidipine, St. John's Wort) are prohibited. In certain embodiments, concomitant use (within 12 days of enrollment) of drugs that are known strong CYP3A4 inducers (such as phenobarbital, rifampin, phenytoin, carbamazepine, rifabutin, rifapentine, clevidipine, St. John's Wort) are permitted. In certain embodiments, concomitant use (within 12 days of enrollment) of drugs that are known strong CYP3 A4 inducers (such as phenobarbital, rifampin, phenytoin, carbamazepine, rifabutin, rifapentine, clevidipine, St. John's Wort) are used with caution.

In certain embodiments, concomitant use (within 12 days of enrollment) of drugs that are substrates or inducers of CYP3A4, inhibitors of CYP3A4 or CYP2C8, substrates of P-glycoprotein (P-gp), breast cancer resistance protein (BCRP) or the MATE1 transporter, substrates of CYP2C19 with narrow therapeutic index are used with caution. In certain embodiments, concomitant use (within 12 days of enrollment) or co-administration of Compound 1 and any one or more drugs that are substrates or inducers of CYP3A4, inhibitors of CYP3A4 or CYP2C8, substrates of P-glycoprotein (P-gp), breast cancer resistance protein (BCRP) or the MATE1 transporter, substrates of CYP2C19 with narrow therapeutic index are permitted. In certain embodiments, concomitant use (within 12 days of enrollment) or co-administration of drugs that are substrates or inducers of CYP3A4, inhibitors of CYP3A4 or CYP2C8, substrates of P-glycoprotein (P-gp), breast cancer resistance protein (BCRP) or the MATE1 transporter, substrates of CYP2C19 with narrow therapeutic index are prohibited.

In certain embodiments, concomitant use (within 12 days of enrollment) or co-administration of stomach acid reducing agents with Compound 1 should be avoided. In certain embodiments, concomitant use (within 12 days of enrollment) or co-administration of stomach acid reducing agents with Compound 1 should be used with caution. In certain embodiments, concomitant use (within 12 days of enrollment) or co-administration of stomach acid reducing agents with Compound 1 is permitted.

Exemplified agents provided herein include, but are not limited to:
CYP3A4 Inducers: phenobarbital, rifampin, phenytoin, carbamazepine, rifabutin, rifapentine, clevidipine, St. John's Wort
CYP3A4 Inhibitors: clarithromycin, erythromycin, diltiazem, itraconazole, ketoconazole, ritonavir, verapamil, Goldenseal, Grapefruit (including juices)
CYP3A4 Substrates (sensitive): buspirone, everolimus, lovastatin, midazolam, simvastatin, triazolam, maraviroc, conivaptan, darifenacin
P-gp Substrates: digoxin, fexofenadine, loperamide, quinidine, talinolol, vinblastine
BCRP Substrates: daidzein, dantrolene, estrone-3-sulfate, prazosin, sulfasalazine
MATE1 Substrates: metformin, tetra-ethylammonium (TEA), cimetidine, procainamide

### Example 5: Phase 1/2 Clinical Study

A FIH, Phase 1/2, multicenter, open-label, dose escalation and expansion study is conducted to evaluate the safety and tolerability of Compound 1, to determine the RP2D and, if applicable, the MTD, and to evaluate the antitumor activity in patients with advanced ROS1-positive NSCLC and other advanced ROS1-positive solid tumors.

### Phase 1 Objectives:

Primary Objective: to determine the RP2D and/or maximum tolerated dose (MTD) of Compound 1 in patients with advanced ROS1-positive solid tumors.

Secondary Objectives: to evaluate the overall safety and tolerability of Compound 1; to characterize the PK profile of Compound 1; to evaluate preliminary antitumor activity of Compound 1 in patients with advanced ROS1 positive solid tumors.

### Phase 2 Objectives:

Primary Objective: to evaluate the ORR of Compound 1 at the RP2D in patients with advanced ROS1-positive NSCLC and other solid tumors.

Secondary Objectives: to assess additional measures of clinical efficacy in patients with ROS1-positive NSCLC and other solid tumors; to evaluate the intracranial antitumor activity of Compound 1 at the RP2D in patients with advanced ROS1-positive NSCLC and other solid tumors; to characterize the safety and tolerability of Compound 1 at the RP2D; to confirm the PK profile of Compound 1 at the RP2D.

### Phase 1 Endpoints:

Primary Endpoint: RP2D and, if applicable, the MTD as determined by incidence of DLTs during Cycle 1, overall safety profile, PK, PD, and preliminary efficacy.

Secondary Endpoints:
Incidence and severity of treatment-emergent adverse events (TEAEs) and changes in clinically relevant laboratory parameters
Pharmacokinetic parameters of Compound 1: Maximum plasma concentration (Cₘₐₓ); Cₘₐₓ DN (Cₘₐₓ-dose normalized), plasma concentration at the end of the dosing interval (Cₜₐᵤ); time of maximum concentration (Tₘₐₓ); area under the curve at the end of the dosing interval (AUCₜₐᵤ); AUC_{tau_DN} (AUCₜₐᵤ-dose normalized), area under the curve from time 0 to 24 (AUC₀₋₂₄); AUC_{0-24_DN} (AUC₀₋₂₄-dose normalized), area under the curve from time 0 to infinity (AUC_{inf}); AUC_{inf_DN} (AUC_{inf}-dose normalized); oral clearance (CL/F); volume of distribution (V_{z}/F); and half-life (t_{1/2})
Objective response rate (ORR) per Response Evaluation Criteria in Solid Tumors Version 1.1 (RECIST 1.1)-Defined as the percent of patients with a complete response (CR) or partial response (PR) according to RECIST 1.1.
Duration of response (DOR) per RECIST 1.1—In responders, defined as the time from first response per RECIST 1.1 to radiographic disease progression or death
Intracranial ORR (IC-ORR) per RECIST 1.1—In patients with measurable metastatic CNS disease at baseline, defined as the proportion of patients with a confirmed intracranial response (IC-CR or IC-PR), based on assessment of up to 5 intracranial target lesions, according to RECIST 1.1 principles per Investigator assessment
Intracranial DOR (IC-DOR) per RECIST 1.1—In patients with intracranial response, defined as the time from first Investigator-assessed IC-response per RECIST 1.1 principles to radiographic IC-disease progression or death
Clinical benefit rate (CBR) per RECIST 1.1-Defined as the percent of patients with a confirmed CR or PR, or stable disease (SD) of at least 24 weeks duration according to RECIST 1.1
Time to response per RECIST 1.1-Defined as the time from first dose to first confirmed radiographic response according to RECIST 1.1
Progression-free survival (PFS) per RECIST 1.1-Defined as the time from first dose to radiographic disease progression per RECIST 1.1 or death

Exploratory Endpoints:
Pharmacodynamic markers associated with response to Compound 1
Extracranial ORR (EC-ORR) per RECIST 1.1
In patients with extracranial metastatic disease, defined as the percent of patients with an extracranial response according to RECIST 1.1 per Investigator assessment
Evaluate metabolite profile of Compound 1

### Phase 2 Endpoints:

Primary Endpoint: ORR per RECIST 1.1-Defined as the proportion of patients with a confirmed CR or PR according to RECIST 1.1 per Blinded Independent Central Review (BICR) of the patients confirmed to have measurable disease at baseline per BICR.

Secondary Endpoints
DOR per RECIST 1.1—In responders, defined as the time from first BICR-assessed confirmed response per RECIST 1.1 to radiographic disease progression or death
IC-ORR per RECIST 1.1—In patients with measurable metastatic CNS disease at baseline, defined as the proportion of patients with a confirmed intracranial response (IC-CR or IC-PR), based on assessment of up to 5 intracranial target lesions according to RECIST 1.1 principles per BICR
IC-DOR per RECIST 1.1—In patients with intracranial response, defined as the time from first BICR-assessed confirmed IC-response per RECIST 1.1 principles to radiographic IC-disease progression or death
CBR per RECIST 1.1-Defined as the percent of patients with a confirmed CR or PR, or SD of at least 24 weeks duration according to RECIST 1.1 per BICR
Time to response per RECIST 1.1—In patients with measurable metastatic CNS disease, defined as the time from first dose to first confirmed radiographic IC-response according to RECIST 1.1 principles per BICR
Time to IC-response per RECIST 1.1—In patients with metastatic CNS disease, defined as the time from first dose to first confirmed radiographic IC-response according to RECIST 1.1 per BICR
Incidence and severity of TEAEs and changes in clinically relevant laboratory parameters
Pharmacokinetic parameters of Compound 1-Cₘₐₓ, C_{max_DN} (Cₘₐₓ dose normalized), Cₜₐᵤ, Tₘₐₓ, AUCₜₐᵤ, AUC_{tau_DN} (AUCₜₐᵤ-dose normalized), AUC₀₋₂₄, AUC₀-24_CN (AUC₀-24 dose normalized), AUC_{inf}, AUC_{inf_DN} (AUC_{inf}-dose normalized), CL/F, V_{z}/F, t_{1/2}
PFS per RECIST 1.1-Defined as the time from first dose to radiographic disease progression per RECIST 1.1 based on BICR assessment or death
Intracranial PFS (IC-PFS) per RECIST 1.1—In patients with measurable metastatic CNS disease, defined as the time from first dose to radiographic disease progression per RECIST 1.1 principles based on BICR assessment or death
Overall survival (OS)-Defined as the time from first dose to death due to any cause

Study Design: The study is conducted in 2 phases (FIG. 1, except n=54 for Phase 1).
Phase 1 employs a Bayesian Optimal Interval Design (BOIN) with 3+3 Run-in Dose Escalation: Patients with advanced/metastatic ROS1-positive NSCLC treated with at least 1 prior ROS1 TKI therapy, or patients with other ROS1-positive solid tumors who have progressed on any prior therapy (includes, but is not limited to, patients who have progressed on prior ROS1 TKIs).
Phase 2 will include 5 cohorts:
   Cohort 2a: Patients with advanced/metastatic ROS1-positive NSCLC naïve to TKI therapy. Up to one prior platinum-based chemotherapy with or without immunotherapy is allowed.
   Cohort 2b: Patients with advanced/metastatic ROS1-positive NSCLC treated with 1 prior ROS1 TKI (either crizotinib or entrectinib) and no prior platinum-based chemotherapy or immunotherapy allowed.
   Cohort 2c: Patients with advanced/metastatic ROS1-positive NSCLC treated with 1 prior ROS1 TKI (either crizotinib or entrectinib) and 1 prior platinum-based chemotherapy with or without immunotherapy.
   Cohort 2d: Patients with advanced/metastatic ROS1-positive NSCLC treated with ≥2 prior ROS1 TKIs and up to 1 prior platinum-based chemotherapy with or without immunotherapy.
   Cohort 2e (exploratory): Patients with any advanced/metastatic ROS1-positive solid tumor (including patients with ROS1-positive NSCLC not otherwise eligible for any other cohorts) and progressed on any prior therapy (includes, but is not limited to, patients who have progressed on prior ROS1 TKIs).

The Phase 1 portion of the study employ a BOIN design with 3+3 run-in to evaluate dose escalation. The primary objective of the Phase 1 portion of the study is to determine the RP2D and, if applicable, the MTD and to assess the safety of Compound 1 in patients with solid tumors. The safety profile is assessed based on physical examinations; ECOG PS; changes from baseline in laboratory parameters, ECGs, vital signs, and ophthalmic examination (baseline only and as clinically indicated); and the reporting of AEs. The DLT Observation Period is defined from first dose on day 1 of cycle 1 (C1D1) through to the end of the first cycle (28-day cycle period).

After patients are treated at a given dose level and monitored for 28 days, available safety data (to include, but not limited to, the incidence of DLT/other AEs and preliminary PK) are reviewed. Initiation of the next dosing group depends on the occurrence of DLT and outcome of the BOIN analysis with consideration to the overall safety profile. During dose escalation, when available data support the safety, PK, and clinical activity of a given dose level, additional patients (up to a total of 12 patients, including patients already included in BOIN dose escalation) may be enrolled at that dose level.

The BOIN dose-escalation is declared complete when the number of evaluable patients treated at the current dose reaches 9 and the decision is to stay at the current dose, or when the maximum sample size is reached. However, if an optimal biologic dose or maximal feasible dose is reached prior to the completion of the BOIN dose-escalation, the RP2D may be declared. The selection of the RP2D occurs after a sufficient number of patients have enrolled and completed the first imaging/efficacy assessment and the data have been reviewed.

In order to further evaluate safety and efficacy and to confirm the RP2D, up to 20 additional patients may be treated at the RP2D, or at one or more candidate RP2Ds, prior to initiation of the Phase 2 portion of the study.

Phase 1 Dose Levels: Phase 1 evaluates ascending doses of Compound 1 monotherapy administered orally once daily (QD) continuously until disease progression, unacceptable toxicity, or voluntary withdrawal of consent. Each cycle of treatment is 28 days. Under the BOIN design, up to approximately 53 patients are enrolled and treated at the dose levels presented in the following table.

| Dose level | Compound 1 Dose | | Frequency* |
|---|---|---|---|
| DL1 | 25 | mg | QD |
| DL2 | 50 | mg | QD |
| DL3 | 75 | mg | QD |
| DL4 | 100 | mg | QD |
| DL5 | 125 | mg | QD |
| DL6 | 150 | mg | QD |

*BID dosing may be explored at any DL.
**Higher DLs may be explored.

Intermediate dose levels may be explored during dose escalation; lower dose levels (e.g., 10 mg QD and 5 mg QD) may be explored as additional dose levels based on results of the BOIN. For example, the following dose reduction levels are provided herein.

| | | |
|---|---|---|
| Initial dose level | Dose level-1 | Dose level-2** |

| | | | | | |
|---|---|---|---|---|---|
| 25 | mg QD | 10 | mg QD | 5 | mg QD |
| 50 | mg QD | 25 | mg QD | 10 | mg QD |
| 75 | mg QD | 50 | mg QD | 25 | mg QD |
| 100 | mg QD | 75 | mg QD | 50 | mg QD |
| 125 | mg QD | 100 | mg QD | 75 | mg QD |
| 150 | mg QD* | 125 | mg QD | 100 | mg QD |

*Higher DLs may be explored. If dose reduction are needed, the dose should be reduced to the lower dose level.
**If treatment is not tolerated after 2 dose reductions, a third dose may be implemented.

Recommended Phase 2 Dose: The recommended Phase 2 dose (RP2D) is expected to be an MTD or a lower optimal biological dose, or a maximum feasible dose, and is the dose level deemed as suitable for testing in the Phase 2 portion of the study. The RP2D is anticipated to achieve sufficient target coverage of the G2032R ROS1 mutation in the periphery and CNS, based on PK modeling simulations; the RP2D also demonstrates a tolerable safety profile along with preliminary antitumor activity. The selection of the RP2D is made using the totality of the data obtained from Phase 1, and includes the evaluation of clinical PK, pharmacodynamics, safety, and antitumor activity.

Duration of Treatment: Across both phases, patients receive study drug continuously from first dose until disease progression, unacceptable toxicity, withdrawal by patient, termination the study, or commercial availability. Patients may continue to receive Compound 1 following progression in the body or brain suitable for local ablation.

End of Study Definition: The end of the study is defined as the date of the last visit of the last patient in the study or termination of the study.

Inclusion Criteria: Patients must meet all of the following criteria to be eligible to enroll in the study:

1. Age ≥18 years.
a. Phase 2 Cohort 2e only: Age >12 years and weighing >40 kg. (Patients ages 12 to 17 are only enrolled in countries and at sites where regulations allow.)
   2. Disease criteria
a. Phase 1: Histologically or cytologically confirmed locally advanced or metastatic solid tumor with documented ROS1 rearrangement determined by testing in a Clinical Laboratory Improvement Amendments (CLIA) laboratory in the US or equivalently accredited diagnostic lab outside the United States (US) and using a local diagnostic test or a commercial test or by a regulatory agency approved test, such as fluorescence in situ hybridization (FISH) or next generation sequencing (NGS) or reverse transcription polymerase chain reaction (RT-PCR).
b. Cohorts 2a, 2b, 2c and 2d: Histologically or cytologically confirmed locally advanced or metastatic NSCLC with ROS1 rearrangement as determined by testing in a CLIA or equivalently accredited diagnostic lab using a local diagnostic test or a commercial test or by a regulatory agency approved test, such as FISH or NGS or RT-PCR.
c. Cohort 2e: Histologically or cytologically confirmed locally advanced or metastatic solid tumor (including NSCLC not eligible for Cohorts 2a-2d) with ROS1 rearrangement as determined by testing in a CLIA or equivalently accredited diagnostic lab using a local diagnostic test or a commercial test or by a regulatory agency approved test, such as FISH or NGS or RT-PCR.
   3. Prior anticancer treatment
a. Phase 1: Patients with ROS1 fusion-positive NSCLC must have previously received at least 1 prior ROS1 TKI, while those with other ROS1-positive solid tumors must have progressed on any prior therapy (includes, but is not limited to, patients who have progressed on prior ROS1 TKIs); any number of prior platinum-based chemotherapies with or without immunotherapy is allowed.
b. Cohort 2a: Must be naïve to TKI therapy and up to one prior platinum-based chemotherapy (with or without immunotherapy).
c. Cohort 2b: Must have received 1 prior ROS1 TKI therapy (either crizotinib or entrectinib) and no prior platinum-based chemotherapy or immunotherapy.
d. Cohort 2c: Must have received 1 prior ROS1 TKI therapy (either crizotinib or entrectinib) and 1 prior platinum-based chemotherapy (with or without immunotherapy)
e. Cohort 2d: Must have received at least 2 prior ROS1 TKI therapies and up to 1 prior platinum-based chemotherapy (with or without immunotherapy).
f. Cohort 2e: Must have progressed on any prior therapy (includes, but is not limited to, patients who have progressed on prior ROS1 TKIs).
   4. Phase 1: Must have evaluable disease (target or nontarget) according to RECIST 1.1. Phase 2: Must have measurable disease, defined as ≥1 radiologically measurable target lesion according to RECIST 1.1 (Eisenhauer et al., Eur. J. Cancer 2009, 45 (2): 228-247). Patients with CNS-only disease are eligible, provided that the disease is evaluable (Phase 1) or measurable (Phase 2) and does not meet Exclusion Criterion #11.
   5. Patient agrees to provide tumor tissue (archived, if available, or a fresh biopsy) for central analysis. It is preferable that submitted tumor tissue be obtained during or after the most recent disease progression. If appropriate tissue is not available, and if biopsy is not considered safe and medically feasible, patient may be approved for enrollment after consultation with medical monitor.
   6. Eastern Cooperative Oncology Group (ECOG) performance status (PS) of 0, 1 or 2 (Oken et al., Am. J. Clin. Oncol. 1982, 5 (6): 649-655).
   7. Adequate organ function and bone marrow reserve as indicated by the following laboratory values on last assessment prior to the first dose of study drug:
a. Bone marrow function: absolute neutrophil count (ANC) ≥1500/μL; platelet count >75,000/μL; hemoglobin ≥8 g/dL (without transfusion).
b. Renal function: estimated creatinine clearance ≥60 mL/min per Cockroft-Gault formula, modified Cockroft-Gault formula, or 24 hour creatinine clearance.
c. Hepatic function: bilirubin <1.5×ULN, unless evidence of Gilbert Syndrome, in which the patient must have total bilirubin <3.0 mg/dL; aspartate aminotransferase and alanine aminotransferase≤3.0×ULN (≤5.0×ULN if liver metastases involvement).
   8. All clinically relevant toxicities related to prior anticancer therapy must have recovered to Grade ≤1 or baseline (except alopecia or ototoxicity).
   9. Women of childbearing potential (WOCBP) and male patients must be surgically sterile or be willing to abstain from sexual activity or use an effective contraceptive method from the time of signing the informed consent form (ICF) through at least 30 days after the last administration of study drug for women and at least 90 days after the last administration of study drug for men (or longer, as required by law or regulation). Effective contraception for WOCBP includes 1 “highly effective method” or 2 “effective” methods based on World Health Organization (WHO) criteria.
   10. Provide written informed consent and willing and able to comply with requirements of the study protocol. Assent must be obtained for patients <18 years old (or for minors who have not reached the age of consent, as defined by local regulations) and a parent/guardian must provide written consent.

Exclusion Criteria: Patients meeting any of the following criteria are excluded from the study:

1. Patient's cancer has a known oncogenic driver alteration other than ROS1. For example, NSCLC with a targetable mutation in EGFR, ALK, MET, RET, or BRAF; colorectal with an oncogenic KRAS, NRAS, or BRAF mutation.
2. Known allergy/hypersensitivity to excipients of Compound 1.
3. Major surgery within 4 weeks of first does of study drug. Minor surgical procedures (e.g., port insertion) are permitted, but with sufficient time for wound healing as deemed clinically appropriate.
4. Ongoing or recent anticancer therapy within the following timeframe prior to first dose of study drug (Compound 1 may be started within limits for prior TKI or chemotherapy if considered to be safe and within the best interest of the patient, with prior approval):
a. TKI or other anti-cancer (excluding chemotherapy, immunotherapy, or cellular therapy for which guidance is given below)<5 half-lives or <7 days, whichever is longer
b. Chemotherapy <21 days
c. Immunotherapy or cellular therapy <28 days
   5. Ongoing or recent radiation therapy within the following timeframe prior to first dose of study drug:
a. Radiation therapy (except palliative radiation to relieve bone pain)<14 days
b. Palliative radiation to relieve bone pain <48 hours
c. Stereotactic or small field brain irradiation <7 days
d. Whole brain radiation <14 days
   6. Prior high-dose chemotherapy requiring stem cell rescue.
   7. Uncontrolled clinically relevant bacterial or fungal infection requiring systemic therapy.
   8. Has known active tuberculosis or active Hepatitis B or C. Active Hepatitis B is defined as a known positive HBsAg result and known quantitative HBV DNA results greater than the lower limits of detection of the assay. Active Hepatitis C is defined by a known positive Hep C Ab result and known quantitative HCV RNA results greater than the lower limits of detection of the assay.
   9. Patient has a QTcF >450 msec (repeated demonstration on more than one assessment). Patient has a history of prolonged QT syndrome or Torsades de pointes.
   10. Patients with clinically significant cardiovascular disease as follows:
a. Within 3 months of enrollment: cerebral vascular accident/stroke; myocardial infarction; unstable angina; uncontrolled atrial fibrillation of any grade
b. History of congestive heart failure (New York Heart Association Classification Class ≥ II); second-degree or third-degree atrioventricular block (unless paced) or any atrioventricular block with PR consistently >220 msec; or ongoing cardiac dysrhythmias of NCI-CTCAE Grade ≥2.
   11. Patient has central nervous system (CNS) metastases or a primary CNS tumor that is associated with progressive neurological symptoms or requires increasing doses of corticosteroids to control the CNS disease. If a patient requires corticosteroids for management of CNS disease, the dose must have been stable for the 2 weeks preceding C1D1. Asymptomatic leptomeningeal carcinomatosis is allowed.
   12. Symptomatic spinal cord compression.
   13. Patients with moderate to severe cognitive impairment or psychiatric disturbances that would compromise the patient's ability to comply with study requirements.
   14. Evidence of active malignancy (other than current ROS1-positive solid malignancy) requiring systemic therapy within the prior 2 years.
a. Exceptions: nonmelanoma skin cancer, in situ melanoma, in situ cervical cancer, papillary thyroid cancer, ductal carcinoma in situ of the breast, or localized and presumed cured prostate cancer. Patients on long-term anti-hormonal therapy for a prior malignancy are allowed as long as the malignancy has not been active within the prior 2 years.
   15. Concomitant use (within 12 days of first dose of study drug) of strong CYP3A4 inducers or strong CYP3 A4 inhibitors.
   16. Manifestation of malabsorption due to prior gastrointestinal surgery, disease, or other illness that could affect oral absorption, distribution, metabolism, or excretion of the study drug.
   17. Patient is pregnant or breast-feeding. WOCBP must have a negative serum pregnancy test at Screening and negative serum or urine tests prior to first dose of study drug.
   18. Actively receiving systemic treatment or direct medical intervention on another therapeutic clinical study.
   19. Any medical condition or laboratory abnormality that would pose a risk to study patient or confound the ability to interpret study results.

Study Drug, Dosage, and Route of Administration: Compound 1 is supplied as film-coated tablets for oral administration in 2 strengths: 5 mg and 25 mg provided in 32-count high-density polyethylene bottles with an induction seal/child resistant cap. Compound 1 should be taken on an empty stomach, at least 1 hour before and no sooner than 2 hours after ingestion of food and/or beverages other than water.

### Prior and Concomitant Medications and Therapies:

Compound 1 was not considered a substrate for CYP2B6, CYP2C9, CYP2C19 or CYP2D6. Compound 1 is a substrate of CYP3A4 with the potential for contributions from CYP1A2 and CYP2C8.

Compound 1 was not considered an inhibitor of CYP1A2, CYP2B6 or CYP2D6 (IC₅₀>100 μM) and was a weak inhibitor of CYP2C8 (IC₅₀=70 μM), CYP2C9 (IC₅₀=70.3 μM), CYP3A4 (testosterone IC₅₀=62.7 μM), CYP2C19 (IC₅₀=38.4 μM) and CYP3A4 (midazolam IC₅₀=30 μM). Compound 1 was a potential inhibitor of CYP3A4 (testosterone) and CYP3A4 (midazolam). Compound 1 was a potential inducer of CYP3A4.

In certain embodiments, concomitant use (within 12 days of enrollment) of drugs that are known strong CYP3A4 inducers (such as phenobarbital, rifampin, phenytoin, carbamazepine, rifabutin, rifapentine, clevidipine, St. John's Wort) are prohibited. In certain embodiments, concomitant use (within 12 days of enrollment) of drugs that are known strong CYP3A4 inducers (such as phenobarbital, rifampin, phenytoin, carbamazepine, rifabutin, rifapentine, clevidipine, St. John's Wort) are permitted. In certain embodiments, concomitant use (within 12 days of enrollment) of drugs that are known strong CYP3A4 inducers (such as phenobarbital, rifampin, phenytoin, carbamazepine, rifabutin, rifapentine, clevidipine, St. John's Wort) are used with caution.

In certain embodiments, concomitant use (within 12 days of enrollment) of drugs that are substrates or inducers of CYP3A4, inhibitors of CYP3A4 or CYP2C8, substrates of P-glycoprotein (P-gp), breast cancer resistance protein (BCRP) or the MATE1 transporter, substrates of CYP2C19 with narrow therapeutic index are used with caution. In certain embodiments, concomitant use (within 12 days of enrollment) or co-administration of Compound 1 and any one or more drugs that are substrates or inducers of CYP3A4, inhibitors of CYP3A4 or CYP2C8, substrates of P-glycoprotein (P-gp), breast cancer resistance protein (BCRP) or the MATE1 transporter, substrates of CYP2C19 with narrow therapeutic index are permitted. In certain embodiments, concomitant use (within 12 days of enrollment) or co-administration of drugs that are substrates or inducers of CYP3A4, inhibitors of CYP3A4 or CYP2C8, substrates of P-glycoprotein (P-gp), breast cancer resistance protein (BCRP) or the MATE1 transporter, substrates of CYP2C19 with narrow therapeutic index are prohibited.

In certain embodiments, concomitant use (within 12 days of enrollment) or co-administration of stomach acid reducing agents with Compound 1 should be avoided. In certain embodiments, concomitant use (within 12 days of enrollment) or co-administration of stomach acid reducing agents with Compound 1 should be used with caution. In certain embodiments, concomitant use (within 12 days of enrollment) or co-administration of stomach acid reducing agents with Compound 1 is permitted.

Exemplified agents provided herein include, but are not limited to:
CYP3A4 Inducers: phenobarbital, rifampin, phenytoin, carbamazepine, rifabutin, rifapentine, clevidipine, St. John's Wort
CYP3A4 Inhibitors: clarithromycin, erythromycin, diltiazem, itraconazole, ketoconazole, ritonavir, verapamil, Goldenseal, Grapefruit (including juices)
CYP3A4 Substrates (sensitive): buspirone, everolimus, lovastatin, midazolam, simvastatin, triazolam, maraviroc, conivaptan, darifenacin
P-gp Substrates: digoxin, fexofenadine, loperamide, quinidine, talinolol, vinblastine
BCRP Substrates: daidzein, dantrolene, estrone-3-sulfate, prazosin, sulfasalazine
MATE1 Substrates: metformin, tetra-ethylammonium (TEA), cimetidine, procainamide

Results. Twenty pts (19 NSCLC, 1 pancreatic cancer) have received Compound 1 orally at dose levels of 25-100 mg once daily. Patients received a median of 3 (range: 1-9) prior anticancer therapies, including any ROS1 TKI (100%); investigational ROS1 TKI (85%, including lorlatinib in 55%, repotrectinib in 40%); ≥2 ROS1 TKIs (75%); any chemotherapy (80%); ≥2 lines of chemotherapy (50%). At baseline, 55% had CNS metastases and 45% had ROS1 G2032R. No DLTs, dose reductions, or drug-related treatment discontinuations have been reported. All treatment-related adverse events (TRAEs) were grade 1. The only TRAE in >1 pt was nausea (n=2). Compound 1 PK analyses demonstrated dose-dependent exposure. Among 12 efficacy-evaluable patients with ROS1+NSCLC treated at 25-75 mg QD, 6 confirmed partial responses (PR) were achieved. Shrinkage or resolution of intracranial metastases were observed; no pts had intracranial progression. A PR was achieved in most (n=5/7) ROS1 G2032R-mutant cancers, including lorlatinib or repotrectinib pretreated tumors. Circulating tumor DNA analyses show reductions of ROS1 variant allele frequency. The RP2D has not been identified and dose escalation continues.

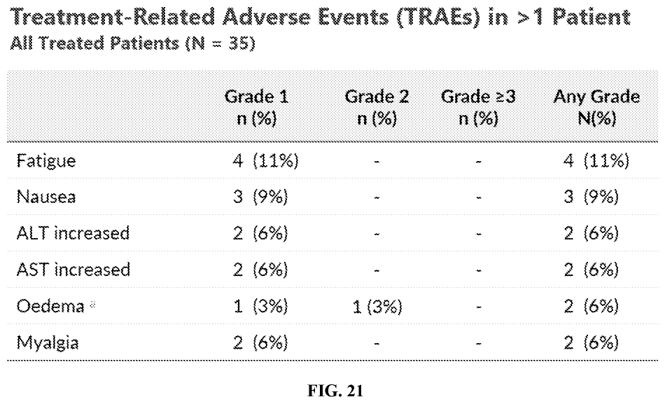

According to the data of Sep. 13, 2022, for patients treated by Sep. 1, 2022, the phase 1 patient population has the heavily pretreated ROS1 positive solid tumors (FIG. 20). The safety profile is favorable and consistent with the highly ROS1-selective, TRK-sparing Compound 1. There is no observed DLTs, no treatment-related SAEs, no AEs leading to dose reduction or discontinuation, no treatment-related dizziness (FIG. 21).

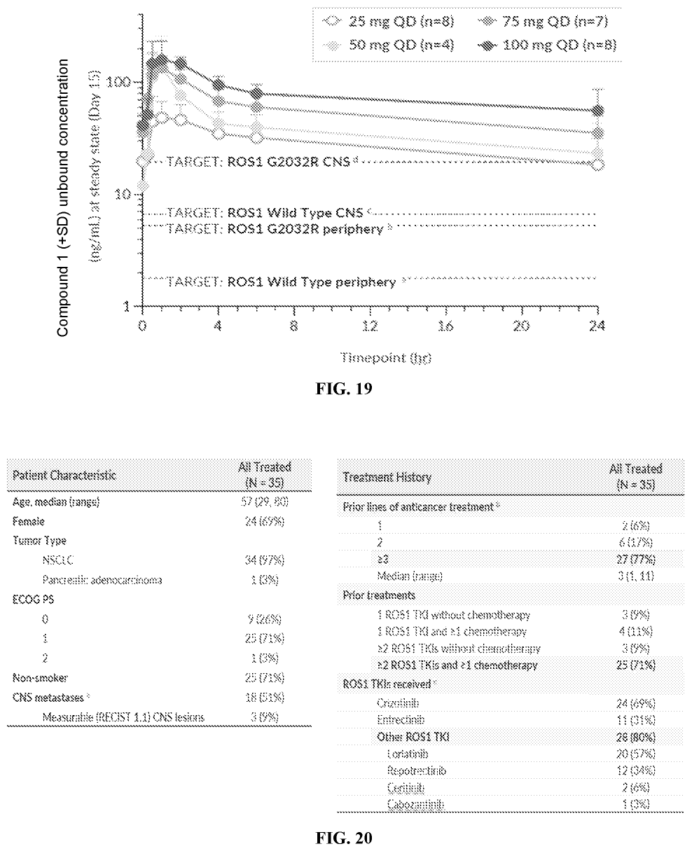

Based on the data as of Sep. 13, 2022, for patients treated by Sep. 1, 2022, Compound 1 exposure in clinical trial exceeded the target levels that provide regression in preclinical models (FIG. 19). Compound 1 demonstrated favorable pharmacokinetics including the low intra-cohort patient PK variability, exposure increasing with increasing dose level, half-life approximately 20h supports QD dosing.

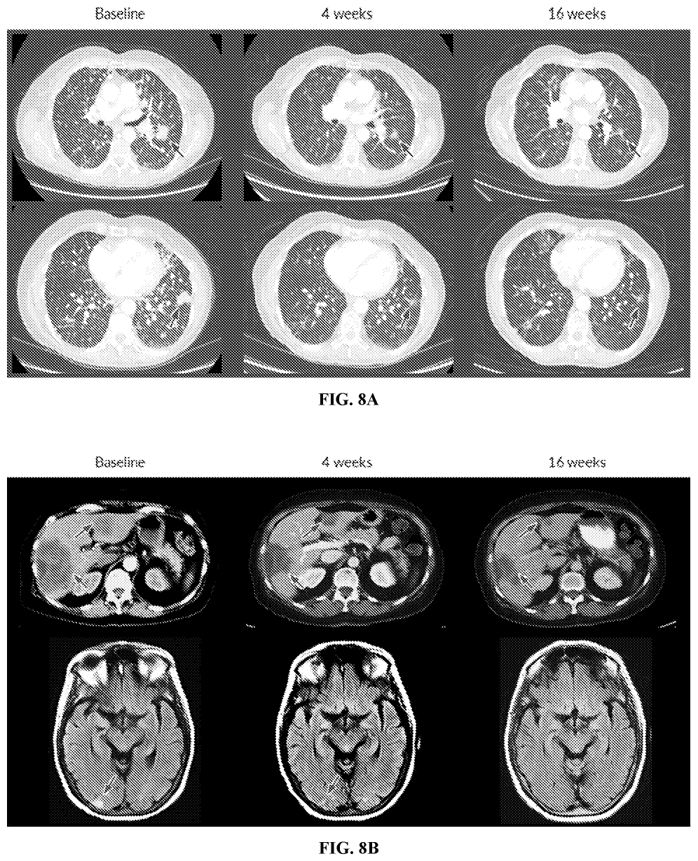

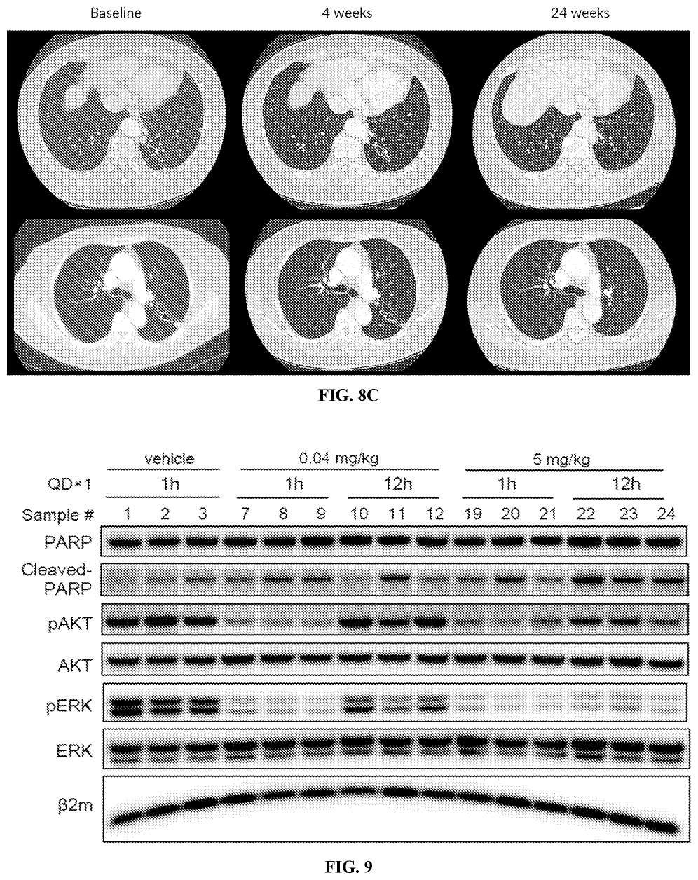

By way of example, a 54-year-old patient with metastatic lung adenocarcinoma that responded to two cycles of carboplatin, paclitaxel, bevacizumab, and atezolizumab tested positive for ROS1 fusion by fluorescence in situ hybridization on tumor tissue. The patient received crizotinib for two months, complicated by pneumonitis, and subsequently received lorlatinib with an initial partial response (PR). Intrathoracic disease progression followed after 32 months. She then received two cycles of carboplatin and pemetrexed with continued lorlatinib and had stable disease. Next-generation sequencing of a lorlatinib-resistant lung nodule revealed a (D) 74-ROS1 fusion with ROS1 G2032R. The patient initiated treatment with Compound 1 at the lowest dose level of 25 mg daily. A clinical response was achieved within 2 weeks of therapy with marked improvement in the patient's dyspnea. Imaging after 4 weeks showed a PR with a decrease in multiple bilateral lung metastases (−31% by RECIST 1.1). Her Compound 1 dose was escalated to 75 mg daily as permitted by the protocol, and she remains on therapy for over 5 months with an ongoing confirmed PR (−60% by RECIST 1.1; FIG. 8A) without neurological toxicities. The unbound plasma concentration of Compound 1 on cycle 1 day 15 over 24 hour period is shown in FIG. 18A.

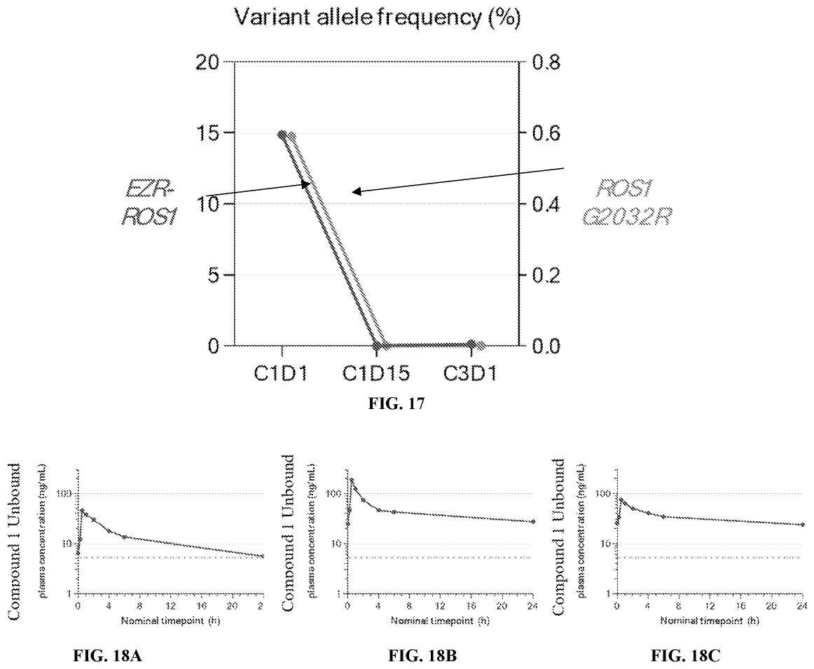

In addition to extracranial antitumor activity, intracranial activity was observed in another patient with metastatic NSCLC harboring EZR-ROS1 G2032R. A 65-year-old patient with stage IV lung adenocarcinoma and multiple brain metastases had EZR-ROS1 identified by ctDNA testing. Entrectinib was initiated with an initial response, but the patient's disease progressed 9 months later. A biopsy of a growing liver lesion confirmed the known EZR-ROS1 fusion and identified a ROS1 G2032R mutation. She was transitioned to a clinical trial of repotrectinib. The patient quickly required a change in systemic therapy due to progressive disease and began a combination of carboplatin, pemetrexed, bevacizumab, and entrectinib. Unfortunately, a mixed response, growing brain metastases, and new liver metastases were observed. Repeat ctDNA testing again detected ROS1 G2032R. Treatment with Compound 1 was initiated at 50 mg daily. At week 4, regression of a right occipital lobe metastasis was observed in addition to a decrease in several metastases in the liver and lung (−38% by RECIST 1.1). She remains on therapy at approximately 5 months with a confirmed PR and further disease regression in the brain and liver (−58% by RECIST 1.1; FIG. 8B). No neurological toxicities were reported. The corresponding ctDNA analysis is shown in FIG. 17 and the unbound plasma concentration of Compound 1 on cycle 1 day 15 over 24 hour period is shown in FIG. 18B.

The antitumor activity of Compound 1 extended to a case without known ROS1 resistance mutations. A 75-year-old patient with a metastatic EZR-ROS1 fusion-positive lung adenocarcinoma was initially treated by a referring physician with alectinib. Primary progression was unsurprisingly observed, and treatment was transitioned to crizotinib. A clinical and radiologic response was maintained until 39 months when bilateral growing lung nodules were noted on imaging. ctDNA analysis did not reveal any ROS1 kinase domain mutations. Treatment with Compound 1 was initiated at 25 mg daily. Treatment was well tolerated with no evidence of dizziness, orthostasis, or paresthesia. A PR (−33% by RECIST 1.1) was observed at week 4; this was confirmed and maintained with ongoing treatment of over 7 months (−48% by RECIST 1.1; FIG. 8C). The unbound plasma concentration of Compound 1 on cycle 1 day 15 over 24 hour period is shown in FIG. 18C.

Compound 1 has been well-tolerated up to 125 mg daily with favorable pharmacokinetics. Activity has been demonstrated in heavily pretreated patients (of whom 70% received ≥2 prior ROS1 TKIs plus chemotherapy), including those with brain metastases and the G2032R mutation.

Whole blood samples were collected at clinical trial sites in STRECK cell-free DNA BCT® tubes and shipped at ambient temperature to central lab for plasma generation and storage at −80° C. Afterwards, the plasma samples were analyzed to determine the genomic alternations in the circulating tumor DNA (ctDNA) via a hybrid capture based next generation sequencing method.

The ctDNA analysis demonstrated that Compound 1 potently suppressed ROS1 variant allele frequency (VAF) at all dose levels tested, compared to the baseline (cycle 1 day 1 “C1D1”). At 25 mg, treatment with Compound 1 led to the complete clearance of both EZR-ROS1 and ROS1 G2032R variants in ctDNA. Complete clearance of ROS1 fusion and G2032R was also observed at higher dose levels. Together, ctDNA data suggests that Compound 1 was active on both ROS1 fusion and ROS1 G2032R solvent front mutation at all dose levels tested, supporting the on-target activity of the compound in patients (FIG. 11). As used in FIG. 11, C1D15 stands for cycle 1 day 15, and C3D1 stands for cycle 3 day 1.

As of the data cutoff of Sep. 19, 2022, the preliminary clinical pharmacokinetics analysis is summarized in the following table:

Parameter (units)

| Dose QD | 25 mg QD | | 50 mg QD | | 75 mg QD | | 100 mg QD | | 125 mg QD | |
|---|---|---|---|---|---|---|---|---|---|---|
| Cycle 1 Day 1 | | | | | | | | | | |
| N, n | 8 | | 4 | | 6 | | 6 | | 3 | |
| Tₘₐₓ (h) | 1.0 | (0.5-1.0) | 1.0 | (0.5-2.0) | 1.0 | (0.5-2.0) | 1.5 | (0.25-4) | 0.5 | (0.5-0.5) |
| Cₘₐₓ (ng/mL) | 199.0 | (24.4) | 337.5 | (69.5) | 641.5 | (36.7) | 802.3 | (109.2) | 943.3 | (73.7) |
| Cₘₐₓ_{_{—}}DN (ng/mL/mg) | 8.0 | (24.2) | 6.8 | (69.5) | 8.6 | (36.7) | 8.0 | (109.2) | 7.5 | (73.7) |
| AUCₗₐₛₜ (hr*ng/mL) | 1360.2 | (36.9) | 3090.5 | (47.1) | 4003.7 | (26.1) | 5724.1 | (35.1) | 5016.4 | (56.3) |
| AUCₗₐₛₜ_{_{—}}DN (hr*ng/mL/mg) | 54.4 | (36.9) | 61.8 | (47.1) | 53.4 | (26.1) | 57.2 | (35.1) | 40.1 | (56.3) |
| AUC_{inf} (hr*ng/mL) | 1964.0 | (52.0) | 4091.1 | (47.2) | 5930.6 | (32.6) | 8688.7 | (44.9) | 5977.8 | (52.8) |
| AUC_{inf}_{_{—}}DN (hr*ng/mL/mg) | 78.6 | (52.0) | 81.8 | (47.2) | 79.1 | (32.6) | 86.9 | (44.9) | 47.8 | (52.8) |
| CL/F (L/hr) | 12.7 | (57.8) | 12.2 | (47.6) | 12.6 | (28.1) | 11.5 | (61.5) | 20.9 | (64.5) |
| Vz/F (L) | 269.9 | (34.3) | 200.1 | (52.1) | 293.8 | (39.6) | 266.9 | (33.6) | 304 | (71.8) |
| t_{1/2} (hr) | 15.5 | (32.3) | 12.0 | (40.2) | 16.4 | (20.8) | 17.0 | (38.1) | 10.1 | (7.3) |
| Cycle 1 Day 15 | | | | | | | | | | |
| N, n | 8 | | 4 | | 6 | | 4 | | 3 | |
| Tₘₐₓ (h) | 1.0 | (0.5-2.0) | 0.5 | (0.5-1.0) | 1.0 | (0.5-2.0) | 0.8 | (0.5-1.0) | 0.5 | (0.5-2.0) |
| Cₘₐₓ (ng/mL) | 249.6 | (41.8) | 687.0 | (141.0) | 729.6 | (24.5) | 818.6 | (28.9) | 733.5 | (16.7) |
| Cₘₐₓ_{_{—}}DN (ng/mL/mg) | 10.0 | (41.8) | 13.7 | (141.0) | 9.7 | (24.5) | 8.2 | (28.9) | 5.9 | (16.7) |
| Cₘᵢₙ (ng/mL) | 67.2 | (102.5) | 83.4 | (117.6) | 186.9 | (87.0) | 296.4 | (56.5) | 141.2 | (79.9) |
| AUCₜₐᵤ (hr*ng/mL) | 2785.2 | (65.0) | 4302.5 | (41.8) | 7010.5 | (51.4) | 6392.6 | (52.1) | 6598.1 | (45.5) |
| AUCₜₐᵤ_{_{—}}DN (hr*ng/mL/mg) | 111.4 | (65.) | 86.6 | (41.8) | 93.5 | (51.4) | 63.9 | (52.1) | 52.8 | (45.5) |
| AUC_{inf} (hr*ng/mL) | 4899.3 | (103.7) | 6074.2 | (58.8) | 13524.6 | (95.2) | 11042.5 | (269.0) | 7503.0 | |
| AUC_{inf}_{_{—}}DN (hr*ng/mL/mg) | 196.0 | (103.7) | 121.5 | (58.8) | 180.3 | (95.2) | 110.4 | (269.0) | 60.0 | |
| CLₛₛ/F (L/hr) | 8.8 | (57.2) | 11.5 | (57.4) | 10.7 | (43.2) | 15.6 | (50.7) | 18.9 | (56.0) |

| Vz/F (L) | 242.9 | (25.4) | 227.7 | (94.9) | 350.0 | (14.0) | 260.9 | (139.0) | 470.4 |
|---|---|---|---|---|---|---|---|---|---|
| t_{1/2} (hr) | 20.3 | (39.6) | 16.0 | (65.7) | 23.6 | (33.4) | 16.4 | | 14.4 |
| R_{ac} | 1.8 | (25.5) | 1.6 | (35.3) | 2.0 | (23.0) | 2.1 | (86.6) | 1.5 |

N = Number of patients in the treatment group, n = number of patients where PK parameters were determined.
A single value is reported when n = 1, a range is reported for n ≥ 2.
The geometric mean (geometric % CV) is shown for all parameters except t_{1/2}, R_{ac} and Tₘₐₓ.
t_{1/2} and R_{ac} = arithmetic mean ± SD, Tₘₐₓ = median (range)
The human free fraction of Compound 1 was calculated as equal to the 20.6% of the total plasma concentration.

As used herein, Cₘₐₓ means maximum plasma concentration; C_{max_DN} means dose normalized Cmax; AUCₗₐₛₜ refers to the area under the plasma concentration time curve from time zero to time of last measurable concentration; AUC_{last_DN} refers to the dose normalized AUCₗₐₛₜ; AUC₀₋₂₄ refers to the area under the curve from time 0 to 24h; AUCₜₐᵤ refers to the area under the curve over the dosing interval; AUC_{tau_DN} refers to the dose normalized AUCₜₐᵤ; AUC_{inf} refers to the area under the curve from time 0 to infinity; AUC_{inf_DN} refers to the dose normalized AUC_{inf}; CL/F refers to the oral clearance; V_{z}/F refers to the volume of distribution (V/F); t_{1/2} refers to the half-life; Cₘᵢₙ refers to the minimum blood plasma concentration reached by a drug over the dosing interval.

Additionally, based on the data cutoff date of Sep. 8, 2022 for patients treated by Sep. 1, 2022, the preliminary efficacy analysis based on the radiographic measurements showed Compound 1 induced tumor response across heavily pretreated patient populations (see the table below). As used herein: CNS refers to the central nervous system; PD refers to progressive disease; PR refers to a confirmed partial response; SD refers to stable disease; TKI refers to a tyrosine kinase inhibitor.

| | | ROS1 G2032R | | ≥2 Prior ROS1 | Prior Lorlatinib |
|---|---|---|---|---|---|
| | All Response - | Resistance | CNS Disease | TKI and ≥1 | and/or |
| NSCLC Response- | Evaluable | Mutation | at Baseline | Chemotherapy | Repotrectinib |
| Evaluable Patients | n = 21 | n = 9 | n = 11 | n = 17 | n = 18 |
| ORR (RESIST | 10 (48%) | 7 (78%) | 8 (73%) | 9 (53%) | 9 (50%) |
| 1.1) | | | | | |
| Best Response | | | | | |
| PR | 10^{a} | 7^{b} | 8a | 9a | 9a |
| SD | 8 | 2 | 2 | 6 | 7 |
| PD | 2 | 0 | 1 | 1 | 1 |
| NE | 19^{c} | 0 | 0 | 1c | 1c |

^{a}Includes 2 ongoing partial responses pending confirmation.
^{b}Includes 1 ongoing partial responses pending confirmation.
^{c}Patient discontinued treatment due to symptomatic deterioration without post-baseline radiographic assessment

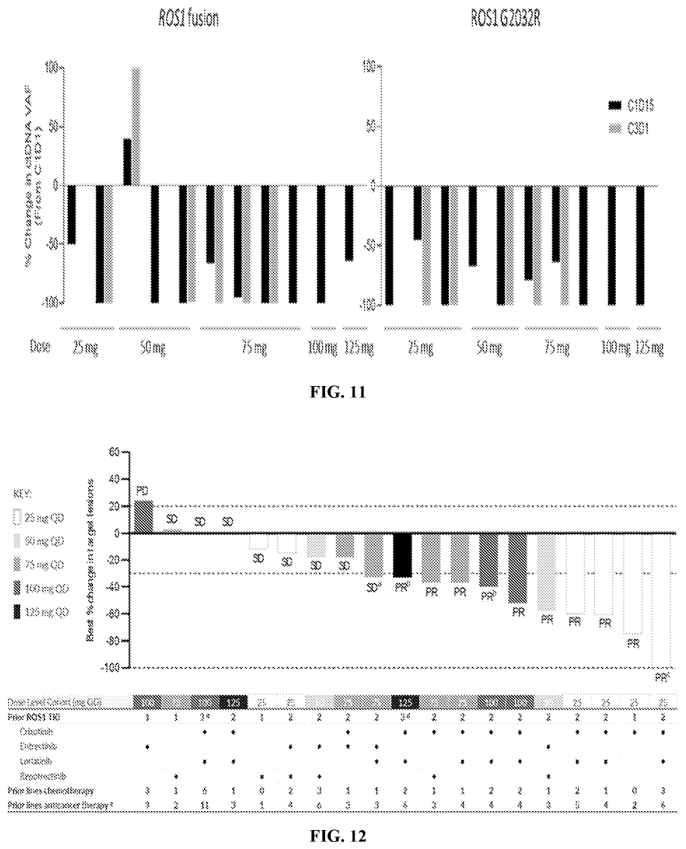

Based on the data cutoff date of Sep. 13, 2022 for the patients treated by Sep. 1, 2022, the preliminary efficacy analysis showed radiographic tumor regression across all dose levels of Compound 1 (see FIG. 12). Two patients (25 mg QD and 125 mg QD dose cohorts, both with prior therapies consisting of crizotinib, lorlatinib and chemotherapy) not shown due to incomplete or missing post-baseline tumor assessments in the setting of PD and symptomatic deterioration.

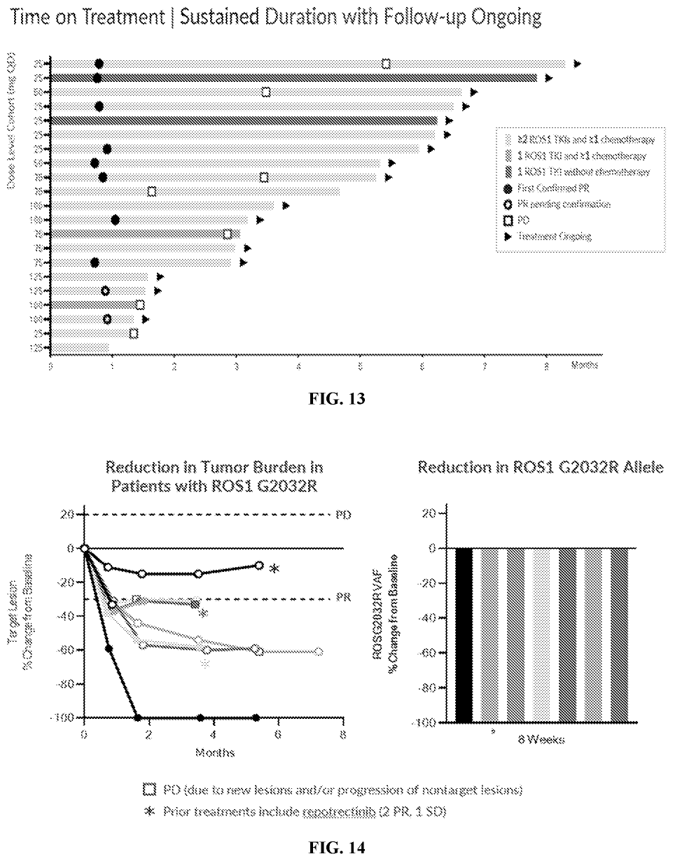

Based on the data as of Sep. 13, 2022, for response-evaluable patients with NSCLC treated by September 1, Compound 1 provided sustained duration of treatment. Median (range) duration of treatment was about 3.6 (1.0, 8.3+) months. No treatment was discontinued due to toxicity. 76% (16/21) response-evaluable patients continue treatment. The median time to response was about 3.6 (range 3.1,4.6) weeks (see FIG. 13).

Based on the data as of Sep. 13, 2022, for response-evaluable patients with NSCLC treated by Sep. 1, 2022, it was observed that Compound 1 induced rapid responses in TKI-resistant patients (see FIG. 14). For the patient subgroup with known ROS1 G2032R resistance mutation, ORR was 78% (7/9) and 100% (9/9) were found to have tumor shrinkage. Complete clearance of G2032R allele in all 7 patients with G2032R detected on central ctDNA analysis. One patient with ROS1 D2033N with ongoing PR (−40%) is pending confirmation.

Based on the data as of Sep. 13, 2022, for patients treated by Sep. 1, 2022, it was found that Compound 1 induced responses in intracranial lesions. Intracranial PR in 3/3 patients with measurable (>10 mm) CNS metastases (one patient with an ongoing intracranial PR is pending confirmation). An ORR of 73% (8/11) was observed in response-evaluable patients with any CNS disease No CNS progression was observed in any of the 35 treated patients.

An intracranial response was observed in a 65-year-old female with CD74-ROS1 fusion NSCLC, previously treated with chemotherapy, crizotinib, and lorlatinib with CNS progression and no known ROS1 resistance mutations (see FIG. 15). The patient continues with treatment with Compound 1 (100 mg QD) at 3.2 months with ongoing response.

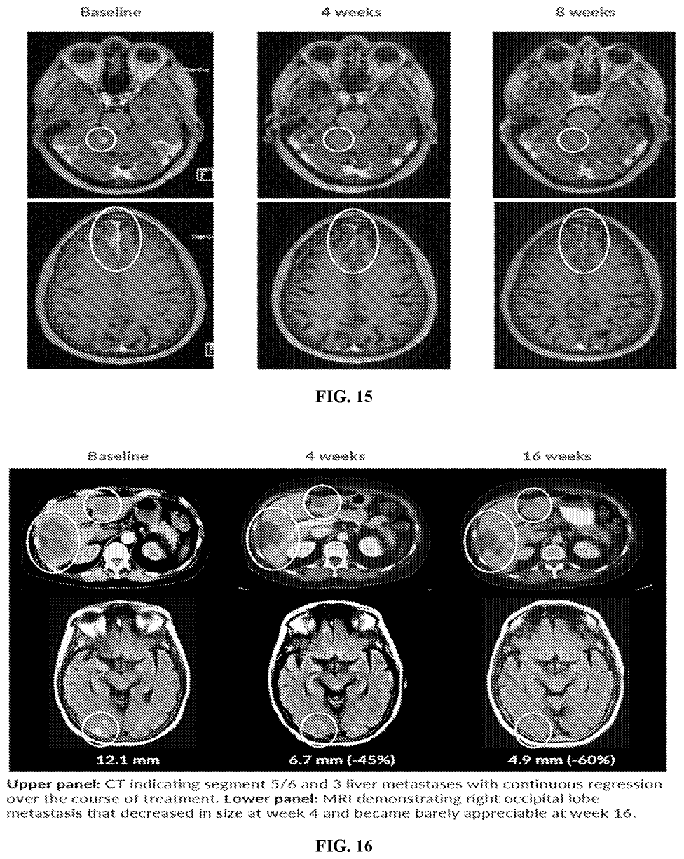

Another case study of treatment with Compound 1 provided the intracranial and extracranial activity in TKI-refractory ROS1 G2032R+NSCLC in a patient diagnosed with EZR-ROS1 fusion NSCLC. The patient had previously been treated with entrectinib (disease progression with ROS1 G2032R identified), and repotrectinib (rapid disease progression observed) The patient had also been previously treated with platinum-based chemotherapy+ bevacizumab+ entrectinib (disease progression observed). Based on the data as of Sep. 13, 2022, it was observed that Compound 1 (50 mg) generated PR (−38% by RECIST 1.1) after 4 weeks, including contraction of right occipital lobe metastasis and decrease in several liver/lung metastases. Further disease regression (−58%) was observed at 16 weeks, including near-complete resolution of brain lesion. The treatment was well-tolerated without dizziness, orthostasis, or paresthesia. The patient continues to receive Compound 1 with ongoing response at 5.3 months. Radiographic images are shown in FIG. 16.

### Example 6: Phase 1 Clinical Study

A phase 1 study is conducted to investigate the food effect, potential impact of pH modification, and drug-drug interaction potential of Compound 1 in healthy subjects. 28 healthy subjects are planned (14 subjects in Part A and 14 subjects in Part B).

Primary Objectives: Part A: to determine the effect of food on the single oral dose PK of Compound 1 in healthy subjects; to determine the impact of multiple oral doses of the PPI lansoprazole (perpetrator molecule), on the single oral dose PK of Compound 1 (victim molecule) in healthy subjects. Part B: to determine the impact of multiple oral doses of Compound 1 (perpetrator molecule) on the single oral dose PK of midazolam (victim molecule) in healthy subjects.

Secondary Objectives: Part A and B: to further assess the safety and tolerability of Compound 1 in healthy subjects.

Primary Endpoints: Part A: pharmacokinetic parameters of Compound 1 determined using non-compartmental analysis (Cₘₐₓ, Tₘₐₓ, AUC₀₋₂₄, AUCₗₐₛₜ, AUC_{inf}, t_{1/2}, CL/F). Part B: pharmacokinetic parameters of midazolam determined using non-compartmental analysis (Cₘₐₓ, AUC₀₋₂₄, AUC_{inf}).

Secondary Endpoints: Parts A and B: incidence and severity of TEAEs, changes in ECG parameters and changes in clinically relevant laboratory parameters.

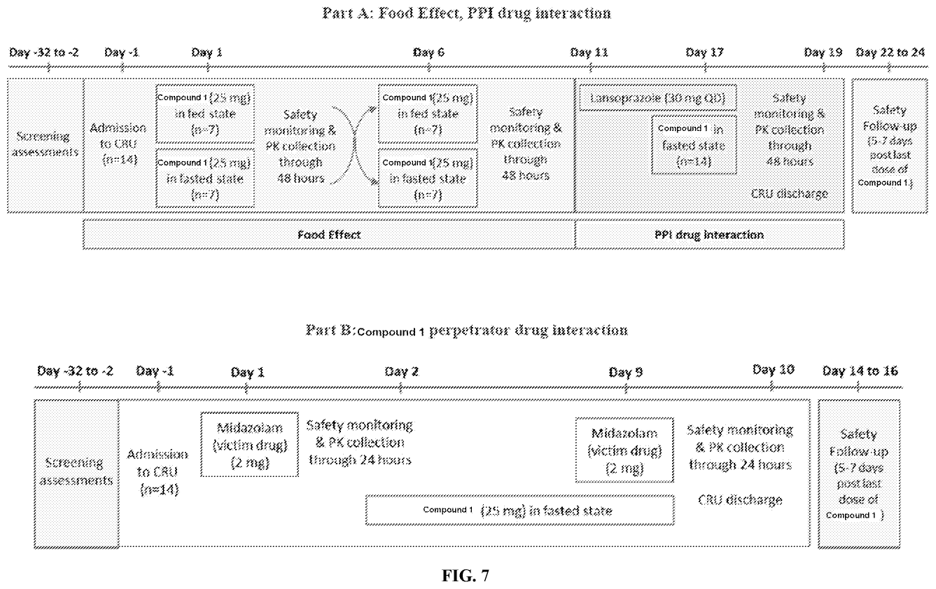

Study Design: The study is conducted in 2 parts (FIG. 7).

This is a Phase 1, open-label study. Part A and Part B are conducted in parallel:

Part A-Food effect, Proton Pump Inhibitor (PPI) drug interaction: Part A utilizes a randomized, balanced, crossover study design. Fourteen (14) healthy subjects are studied in a single cohort as follows:
Subjects reside in the CRU from Day-1 to Day 19.
In the food-effect portion of this study, each subject receives single oral doses of Compound 1 (25 mg) at either fed (high fat breakfast) or fasted status on Days 1 and 6. Half of the subjects (n=7) are randomized to receive Compound 1 under fed conditions on Day 1 then cross over to receive Compound 1 under fasted conditions on Day 6, and half (n=7) are randomized to receive Compound 1 under fasted conditions on Day 1 then cross over to receive Compound 1 under fed conditions on Day 6.
In the PPI drug interaction portion of this study, subjects receive lansoprazole (30 mg QD) from Day 11 to Day 17, followed by a third dose of Compound 1 (25 mg) on Day 17 under fasted conditions. Post-dose PK sampling continues to 48 hours (the morning of Day 19).
All subjects have a follow-up (clinic visit or phone call) 5-7 days after the final Compound 1 dose to review safety.

Part B-Compound 1 perpetrator drug-drug interaction: Part B utilizes a fixed sequence, multiple oral Compound 1 dose, study design. Fourteen (14) healthy subjects are studied as a single group:
Subjects reside in the CRU from Day-1 until the morning of Day 10.
On Days 1 and 9, midazolam (2 mg) is administered and PK timepoints are collected over 24 hours. Compound 1 (25 mg QD) is administered on Days 2 through 9 under fasting conditions.
All subjects have a follow-up (clinic visit or phone call) 5-7 days after the final Compound 1 dose to review safety.

Duration of Treatment: Subjects in Part A receive treatment for 17 days: three single daily doses of Compound 1 (on study Days 1, 6 and 17), as well as lansoprazole from Day 11 to Day 17. Subjects in Part B receive treatment for 9 days: daily Compound 1 dosing (study Days 2-9), as well as midazolam on Days 1 and 9.

Inclusion Criteria: Subjects must meet all of the following criteria to be eligible to enroll in the study:

1. Healthy males and females.
2. Age 18 to 55 years inclusive.
3. BMI 18 to 32 kg/m2 inclusive.
4. Any ethnic origin.

### 5. Non-smokers.

6. Women of childbearing potential and male subjects must be surgically sterile or be willing to abstain from sexual activity or use an effective contraceptive method from the time of signing the ICF through 30 days after the last administration of study drug for women and 90 days after the last administration of study drug for men.
7. Male subjects must agree to not donate sperm throughout the study and for 90 days following the last dose of Compound 1.
8. Provide written informed consent and be willing and able to comply with requirements of the study protocol, including but not limited to the study restrictions outlined regarding food consumption, alcohol, caffeine, tobacco/nicotine, recreational drug use, exercise, and donation of sperm (for male subjects) and/or blood, plasma or platelets.

Exclusion criteria: Subjects meeting any of the following criteria are excluded from the study:

1. History of significant hypersensitivity, intolerance, or allergy to any drug compound, food, or other substance, or any contraindication to lansoprazole (Part A) or midazolam (Part B), unless approved by the Investigator (or designee).
2. Hospitalization within 2 months prior to admission to CRU, major surgical procedure of any type within 6 months.
3. History of chronic or recurrent infections considered a risk by the Investigator; a serious or life-threatening infection within the 6 months prior to admission to CRU; or any suspected current active bacterial or viral infection requiring antibiotics, antivirals or other aggressive treatment or considered to be a risk to study participation by the Investigator.
4. Has known active tuberculosis, positive HIV test, positive hepatitis B panel, and/or hepatitis C antibody. Subjects whose hepatitis B results are compatible with prior immunization may be included.
5. Subject has a QTcF consistently >450 msec (in males) or >470 msec (in females). In the event of an out-of-range value, the ECG may be repeated twice and the mean of the 3 results may be used to determine whether the subject is excluded. Subject has a history of prolonged QT syndrome or Torsades de pointes.
6. Subjects with moderate to severe cognitive impairment or psychiatric disturbances that would compromise the subject's ability to comply with study requirements.
7. Subjects who have received:
a. Herbal medications (such as St. John's wort) or vaccines (including COVID vaccine and/or booster) within 30 days prior to admission to CRU.
b. OTC medications, multivitamins or homeopathic preparations within 7 days prior to admission to CRU.
c. Any other medications (including prescription medications, oral contraceptives, or other hormonal contraceptive treatments) within 14 days prior to admission to CRU.
d. NOTE: Permitted exceptions include occasional use of acetaminophen up to 2 grams per day for no more than 3 consecutive days and other medications where ≥ 5 half-lives have elapsed since last dose (upon approval by the Investigator).
   8. Self-reported drug or alcohol abuse and/or dependence within 1 year prior to admission to CRU, including subjects who have been in a drug or alcohol rehabilitation program within 1 year prior to admission to CRU.
   9. Active alcohol consumption of more than 14 units per week or consumption of alcohol within 48 hours prior to admission to CRU.
   10. Use of any tobacco or nicotine containing substances for at least 3 months prior to admission to CRU through the final discharge from the CRU.
   11. Positive test results for drugs of abuse, alcohol, or cotinine at screening or admission to CRU.
   12. Consumption of caffeine within 48 hours prior to admission to CRU.
   13. Consumption of foods and beverages containing grapefruit within 7 days prior to admission to CRU.
   14. Donation or loss of >500 mL whole blood within 30 days prior to admission to CRU; donation of plasma within 2 weeks prior to admission to CRU; or donation of platelets within 6 weeks prior to admission to CRU.
   15. Participation in strenuous exercise within 72 hours prior to admission to the CRU, and agrees not to begin a new exercise program nor participate in any unusually strenuous physical exertion throughout study participation.
   16. Subject is pregnant or breastfeeding. Female subjects must have a negative serum pregnancy test at Screening and at check-in to the CRU. A positive BHcG test is exclusionary unless determined consistent with post-menopausal status upon quantitative testing.
   17. Actively enrolled in another clinical study involving an investigational drug.
   18. Any of the following laboratory abnormalities at screening or at check-in to the CRU (repeat testing may be performed for confirmation):
a. Total bilirubin >1.5×ULN (NOTE: subjects with known or suspected Gilbert's syndrome are excluded.)
   b. AST, ALT, or alkaline phosphatase >2×ULN
      19. Prior gastrointestinal surgery (excluding uncomplicated appendectomy, uncomplicated cholecystectomy, or hernia repair or cosmetic surgery such as abdominoplasty), disease, or other illness that could potentially affect oral absorption, distribution, metabolism, or excretion of the study drug as judged by Investigator.
      20. Any other medical condition or laboratory abnormality or other reason that in the opinion of the Investigator would pose a risk to study subject or confound the ability to interpret study results.

Study Drug, Dosage, and Route of Administration: Compound 1 is supplied as tablets for oral administration in 1 strength: 25 mg (oblong) provided in 32-count high-density polyethylene bottles with an induction seal/child resistant cap.

Study Drug, Dosage, and Route of Administration: Subjects should refrain from taking any medications during the course of the study. Any medication which is considered necessary for the subject's safety and well-being may be given at the discretion of the Investigator, with consideration to the below guidance. The administration of all medications (including investigational products) must be listed on the appropriate CRF page.

Medications to Avoid or Take with Caution: The following medications, supplements, and foods must be avoided or taken with caution (exemplified agents are provided in Example 4):
Medications that are strong inhibitors of CYP3A4.
Herbal supplements and foods that are strong inhibitors of CYP3A4 including, but not limited to, grapefruit and grapefruit juice.
Medications that are known substrates of CYP3A4.
Medications that are CYP2C19 substrates with a narrow therapeutic index.
Medications that are known substrates of P-gp/MDR1 and BCRP/ABCG2.
Medications that are known substrates of the MATE1 transporter (including but not limited to metformin, cimetidine, procainamide, etc.).
Stomach acid reducing agents, such as PPI. Co-administration of stomach acid reducing agents with Compound 1 should be avoided, as the potential exists for drug-drug interactions between Compound 1 and stomach acid reducing agents.
Warfarin and low molecular weight heparin.

Prohibited Medications and Therapies: The following medications and therapies must be excluded during the study:
Any other investigational therapy.
Medications that are strong inducers of CYP3A4

### Statistical Methods:

Analysis Sets: The PK analysis set is defined as all subjects who receive study treatment and have at least one evaluable plasma PK sample. PK is examined among the following subject subsets: Part A: Compound 1 PK in fed subjects (n=14); Compound 1 PK in fasted subjects (n=14); Compound 1 PK in subjects receiving lansoprazole (PPI) to steady state (n=14). Part B: Midazolam PK (n=14); Midazolam PK in subjects receiving Compound 1 to steady state (n=14). The safety analysis set consists of subjects who receive at least 1 dose of Compound 1.

Analysis of Pharmacokinetics: PK parameters are determined using Phoenix 64 (Certara, version 8.2) and a noncompartmental approach. All PK concentration and parameter analyses are conducted on the PK analysis set. Terminal elimination half-life are estimated using a minimum of three timepoints, after the timepoint at which Cₘₐₓ is established. For plots and derivation of PK parameters, below limit of quantitation values that occur before the first measurable concentration are set to zero. Missing values that occur after the first measurable concentration are set to missing. Actual sampling times are used. The actual post dose sampling times are expressed in hours and rounded off to three significant figures.

Derived PK parameters: Cₘₐₓ: maximum observed plasma concentration. Tₘₐₓ: Time to reach Cₘₐₓ. AUC₀₋₂₄: Area under the plasma concentration time curve, from time of dosing to 24 hours post dose. AUCₗₐₛₜ: Area under the plasma concentration time curve, from time of dosing to the last measurable non-zero concentration. AUC_{inf}. Area under the plasma concentration time curve, from time of dosing to infinity. t_{1/2}: terminal elimination half-life. CL/F: Apparent total clearance of drug from plasma following oral administration.

Summary of PK parameters: Derived PK parameters are summarized using the number of observations (n) and the following statistics: mean, median, standard deviation, coefficient of variation, minimum, maximum, geometric mean.

### Comparison of PK Parameters:

Part A: Food Effect: geometric mean (% CV) of Cₘₐₓ, AUCₗₐₛₜ and AUC_{inf} for Compound 1 in subjects at fed and fasted status are calculated. The data are presented as the geometric mean ratio of fed versus fasted.

Part A: PPI drug interaction: geometric mean (% CV) of Cₘₐₓ, AUCₗₐₛₜ and AUC_{inf} for Compound 1 in subjects at fasted status and subject at fasted status receiving lansoprazole (PPI) at steady state are calculated. The data are presented as the geometric mean ratio of Compound 1 alone versus Compound 1+ lansoprazole.

Part B: Compound 1 perpetrator DDIL: geometric mean (% CV) of Cₘₐₓ, AUC₀-24, AUC_{inf}, for midazolam in subjects prior to receiving Compound 1 and following administration of Compound 1 at steady state are calculated. The data are presented as the geometric mean ratio of midazolam alone versus midazolam+ Compound 1.

Other derived PK parameters can be evaluated and included in the data set as needed or deemed useful in the analysis.

Preliminary analysis of the Part A study:

Preliminary analysis of the data as of Sep. 21, 2022 found the geometric mean ratio of AUC₀-inf is about 1.12; the geometric mean ratio of AUC₀₋₄₈ is about 1.08; the geometric mean ratio of Cₘₐₓ is about 0.84. The geometric mean ratios were calculated as the value (e.g. Cₘₐₓ, AUC₀₋₄₈ and AUC_{inf}) for subjects in a fed versus those in a fasted status. The preliminary results suggest that compound 1 may be taken with or without food.

Preliminary analysis of the data as of Sep. 21, 2022 found the geometric mean ratio of AUC₀-inf is about 0.92; the geometric mean ratio of AUC₀₋₄₈ is about 0.92; the geometric mean ratio of Cₘₐₓ is about 0.73. The geometric mean ratios were calculated as the value (e.g. Cₘₐₓ, AUC₀₋₄₈ and AUC_{inf}) for subjects receiving both Compound 1 and lansoprazole versus those receiving Compound 1 alone. The preliminary results suggest that compound 1 may be taken concomitantly with a PPI.

Preliminary analysis of the Part B study:

Preliminary analysis of the data as of Sep. 21, 2022 found the geometric mean ratio of AUC₀-inf is about 1.12; the geometric mean ratio of AUC₀₋₂₄ is about 1.11; the geometric mean ratio of Cₘₐₓ is about 1.20. The geometric mean ratios were calculated as the value (e.g. Cₘₐₓ, AUC₀₋₄₈ and AUC_{inf}) for subjects receiving midazolam alone versus those receiving both midazolam and Compound 1. The preliminary results suggest that compound 1 may not be concomitant taken with a sensitive CYP3A4 substrate.

### Example 7: Cellular Viability Assay

Cell Culture: All cells were maintained at 37° C. with 5% CO₂. Ba/F3 cells were provided by the RIKEN BRC through the National Bio-Resource Project off the MEXT, Japan. Ba/F3 cells were maintained in RPMI-1640+10% FBS. Genes encoding human CD74-ROS1 (with wild-type kinase domain or with G2032R, D2033N, L2026M, or S1986F mutation), TPM3-TRKA, ETV6-TRKB, ETV6-TRKC, or TRKB (full-length) were synthesized, cloned into a retroviral vector with a puromycin-resistance marker, and packaged into retroviral particles. The virus was used to infect Ba/F3 cells. Stable cell lines were selected by interleukin (IL)-3 withdrawal and with puromycin for at least 7 days. The polyclonal culture was used in assays directly, or monoclonal cultures were established through limiting dilution before being used in assays. Successful transformants were confirmed by Sanger sequencing and western blot. All cells were confirmed to express the full desired protein. Ba/F3 CD74-ROS1 contained a small C-terminal truncation, and Ba/F3 ETV6-TRKC contained a small C-terminal frameshift. Both were confirmed by orthogonal assays to have no discernable effects on inhibitor activity. HCC78 and A549 were obtained at Pharmaron and cultured in 1640 medium+10% FBS or F12K medium+10% FBS, respectively.

Alternatively, Ba/F3 cells expressing the following genes were made separately. ROS1 fusion mutants were made using site-directed mutagenesis (Agilent, New England Biolabs). Platinum-E cells (Cell Biolabs, Inc) were transfected with pBABE CD74-ROS1, pMIG CEP85L-ROS1, pCX4 EZR-ROS1, pBABE GOPC(S)—ROS1, pCX4 GOPC(S)—ROS1, pBABE GOPC (L)-ROS1, pCX4 GOPC (L)-ROS1, or pMIG SLC34A2-ROS1 wild-type or mutant constructs using Biotool DNA transfection reagent to generate replication incompetent, ecotropic retrovirus. Ba/F3 parental cells were infected with retrovirus. Cells were treated with puromycin to select for cells stably expressing the respective fusions. Cells that survived IL-3 withdrawal were used for in vitro assays. All transformed cell lines were sequenced to verify the presence of desired mutations. Cells were harvested, pelleted and DNA was extracted using QuickExtract™ DNA Extraction Solution (Lucigen). The ROS1 kinase and C-terminal domains were PCR amplified. Benchling software was used to align chromatographs to confirm the presence of desired mutations and verify that no undesired mutations were introduced during viral transduction.

Cell Viability Assay: A549 or stable Ba/F3 cells were seeded into 384-well plates, and test compounds were added in a 3-fold dilution series in complete culture medium containing 10% FBS. After a 72-hour incubation with the inhibitor, cell viability was measured using the CellTiter-Glo reagent (Promega). Untreated wells served as negative controls (no inhibition of proliferation), whereas wells treated with high concentrations of the non-specific kinase inhibitor staurosporine served as positive controls (full inhibition of proliferation). IC₅₀ was calculated from percent inhibition and log (inhibitor concentration) using 4-parameter logistic regression.

Alternatively, all inhibitors were prepared as 1 mM stocks in DMSO. Plates were pre-seeded with 25 μL per well of complete medium using a Multidrop Combi Reagent Dispenser (Thermo Scientific). Inhibitors were distributed onto 384-well plates at 2-fold of the indicated concentrations into 25 μL per well of complete medium using a D300 Digital Dispenser (Hewlett-Packard). Ba/F3 cell lines expressing wild-type or mutant ROS1 fusions were seeded at 1,000 cells per well in a volume of 25 μL using Multidrop Combi Reagent Dispenser (Thermo Scientific). Plates were incubated for 72 hours. Viability was measured using a WST-8 [2-(2-methoxy-4-nitrophenyl)-3-(4-nitrophenyl)-5-(2,4-disulfophenyl)-2H-tetrazolium, monosodium salt] based assay (Bimake) and read on a Biotek Synergy 2 plate reader. Each condition was assayed in triplicate. Data were normalized using Microsoft Excel, and IC₅₀ values were calculated using a nonlinear regression analysis in GraphPad Prism.

Results: Six ROS1 TKIs (crizotinib, entrectinib, lorlatinib, taletrectinib, repotrectinib, and Compound 1) were profiled in cell viability assays against one human cancer cell line and 18 engineered Ba/F3 cell lines. These cell lines encompassed six ROS1 fusion partners (SLC34A2, EZR, CD74, GOPC (L), GOPC(S), and CEP85L) and eight ROS1 mutational variants (wild-type, G2032R, S1986F, F2004C, F2004V, L2026M, D2033N, and G2101A).

In seven cell lines expressing wild-type ROS1 fusions, all six TKIs had appreciable growth inhibitory activity albeit with differing levels of potency. Compound 1 and lorlatinib were the most potent (average IC₅₀=0.4 nM and 0.5 nM, respectively), with potency exceeding that of repotrectinib by 7-fold (IC₅₀=3.3 nM) and that of crizotinib, entrectinib, or taletrectinib by >19-fold (IC₅₀=9.7-30 nM).

In six cell lines harboring ROS1 fusions with the G2032R mutation, Compound 1 was the only TKI to achieve single-digit nanomolar potency (average IC₅₀=1.6 nM), which exceeded repotrectinib and taletrectinib by >10-fold (IC₅₀=18-44 nM), lorlatinib by >60-fold (IC₅₀=98 nM), and crizotinib and entrectinib by >500-fold (IC₅₀=846 nM-916 nM).

IC₅₀ (nM) results of Compound 1 and several ROS1 inhibitors are shown below.

| Model | | | | | | | |
|---|---|---|---|---|---|---|---|
| name | ROS1 Status | Cmpd 1 | Crizotinib | Entrectinib | Lorlatinib | Taletrectinib | Repotrectinib |
| HCC78 | SLC34A2- | <0.7 | 45 | 5.3 | 0.9 | 15 | 7.7 |
| | ROS1 | | | | | | |
| Ba/F3 | CD74-ROS1 | 1.3 | 40 | 19 | 1.2 | 21 | 4.6 |
| Ba/F3 | EZR-ROS1 | 0.3 | 5.9 | 12 | 0.5 | 11 | 2.9 |
| Ba/F3 | GOPC(L)- | 0.2 | 33 | 11 | 0.3 | 7.6 | 3.5 |
| | ROS1 | | | | | | |
| Ba/F3 | GOPC(S)- | 0.2 | 110 | 36 | 0.3 | 9.9 | 2.7 |
| | ROS1 | | | | | | |
| Ba/F3 | CEP85L- | 0.2 | 30 | 13 | 0.4 | 6.2 | 2.1 |
| | ROS1 | | | | | | |
| Ba/F3 | CD74-ROS1 | 3.6 | 950 | 880 | 320 | 84 | 25 |
| | G2032R | | | | | | |
| Ba/F3 | EZR-ROS1 | 0.7 | 630 | 830 | 42 | 19 | 8.8 |
| | G2032R | | | | | | |
| Ba/F3 | GOPC(L)- | 1.1 | 1600 | 1200 | 91 | 27 | 11 |
| | ROS1 | | | | | | |
| | G2032R | | | | | | |
| Ba/F3 | GOPC(S)- | 6.6 | 1200 | >3000 | >100 | >100 | >100 |
| | ROS1 | | | | | | |
| | G2032R | | | | | | |
| Ba/F3 | SLC34A2- | 0.2 | 440 | 220 | 14 | 9.5 | 3.8 |
| | ROS1 | | | | | | |
| | G2032R | | | | | | |

To measure the effects of G2032R substitution on TKI sensitivity, the IC₅₀ shifts from wild-type ROS1 to ROS1 G2032R were analyzed for six TKIs in three matched fusion pairs (CD74-ROS1, EZR-ROS1, and GOPC (L)-ROS1 cell lines, wt and G2032R-positive). The analysis revealed two distinct groups of ROS1 TKIs based on their tolerance to G2032R. For the first group comprising crizotinib, entrectinib, and lorlatinib, G2032R was detrimental, causing an average IC₅₀ loss of 60-220-fold compared to wild-type ROS1 and pushing the G2032R potency outside a desirable range. The second group comprising taletrectinib, repotrectinib, and Compound 1 was tolerant of G2032R, showing only a modest IC₅₀ loss of 3-4-fold compared to wild-type ROS1. In contrast to repotrectinib and taletrectinib, however, Compound 1 accommodated the 4-fold shift for ROS1 G2032R and still maintained single-digit nanomolar potency due to its sub-nanomolar potency against wild-type ROS1.

Besides G2032R, other substitutions observed after disease progression on crizotinib and/or entrectinib include S1986F, F2004C, F2004V, L2026M, D2033N, and G2101A. CD74-ROS1 or EZR-ROS1 fusion proteins harboring these mutations were expressed in Ba/F3 cells and tested in cell viability assays against six TKIs. Compound 1 potently inhibited the non-G2032R ROS1 mutants with IC₅₀≤1.5 nM. IC₅₀ shift analysis revealed that most non-G2032R mutations caused ROS1 to become more resistant to crizotinib and entrectinib, but the degree of resistance was modest (IC₅₀ shift=0.7-5.7-fold) and smaller in magnitude compared to G2032R (IC₅₀ shift=24-110-fold). Conversely, the non-G2032R mutations did not confer resistance to lorlatinib, taletrectinib, repotrectinib, and Compound 1 (IC₅₀ shift=0.01-1.3-fold).

IC₅₀ (nM) results of Compound 1 and several ROS1 inhibitors are shown below.

| Model | | | | | | | |
|---|---|---|---|---|---|---|---|
| name | ROS1 Status | Cmpd 1 | Crizotinib | Entrectinib | Lorlatinib | Taletrectinib | Repotrectinib |
| Ba/F3 | CD74-ROS1 | <0.6 | 39 | 26 | <0.3 | NA | 0.84 |
| | S1987F | | | | | | |
| Ba/F3 | CD74-ROS1 | 0.02 | 35 | 60 | 0.3 | 13 | 3.2 |
| | F2004C | | | | | | |
| Ba/F3 | EZR-ROS1 | 0.01 | 28 | 66 | 0.5 | 13 | 3.5 |
| | F2004C | | | | | | |
| Ba/F3 | CD74-ROS1 | 0.01 | 35 | 38 | 0.5 | 8.5 | 2.5 |
| | F2004V | | | | | | |
| Ba/F3 | EZR-ROS1 | 0.2 | 11 | 51 | 0.6 | 10 | 3.6 |
| | F2004V | | | | | | |
| Ba/F3 | CD74-ROS1 | 1.5 | 110 | 41 | 0.8 | NA | 3.3 |
| | L2026M | | | | | | |
| Ba/F3 | CD74-ROS1 | 1 | 77 | 79 | 0.4 | NA | 2.5 |
| | D2033N | | | | | | |
| Ba/F3 | EZR-ROS1 | 0.4 | 25 | 8.4 | 0.4 | 9.3 | 1.8 |
| | G2101A | | | | | | |

Given the diversity of upstream ROS1 fusion partners, ROS1 TKIs were interrogated in Ba/F3 cells expressing CD74-, EZR-, GOPC (L)-, GOPC(S)—, CEP85L-, or SLC34A2-ROS1 fusions, with a wild-type kinase domain or with the G2032R mutation. Compound 1 showed potent activity across all ROS1 fusions evaluated (IC₅₀<10 nM).

### Example 8: Anchorage Independent Growth

Summary: Anoikis avoidance is a hallmark of cancer and is potentially indicative of migratory or metastatic propensity. To study the effect of ROS1 inhibition on anoikis, anchorage-independent colony formation assays in NIH3T3 cells expressing CD74-ROS1 or EZR-ROS1 fusions were performed.

Colony Formation Assay: Plates were pre-seeded with 0.8% agarose in complete medium with either DMSO or inhibitor (crizotinib, entrectinib, lorlatinib, or Compound 1 at 10, 100, or 1000 nM). Each inhibitor was paired with its own DMSO condition to serve as an accurate control. NIH3T3 cells expressing CD74-ROS1 or EZR-ROS1 wild-type or mutant fusions were plated in 0.4% agarose in complete medium at a density of 2,000 cells per 0.5 mL of agarose with DMSO or inhibitor at identical concentration to the bottom layer. Plates were incubated for 4 weeks, and each well was fed 3× per week with 75 μL of complete medium with or without inhibitor to match each plated condition to prevent drying of the agarose. Plates were read after 3 and 4 weeks using a GelCount™ (Oxford Optronix). Colony counts were averaged by condition and normalized to the colony counts from paired DMSO conditions. Data analysis and visualization was performed using Microsoft Excel and GraphPad Prism.

Results: ROS1-transformed NIH3T3 cells lost contact inhibition and formed colonies on soft agar. For NIH3T3 cells expressing wild-type ROS1 fusions, all ROS1 TKIs evaluated suppressed colony formation by >80% at <100 nM, with Compound 1 and lorlatinib having the highest potency, followed by crizotinib and then entrectinib. This potency trend correlated with the degree of target engagement and signaling modulation measured by western blotting. In contrast, for NIH3T3 cells expressing ROS1 fusions with G2032R, only Compound 1 was able to inhibit colony formation by >90% at 100 nM, whereas other TKIs (crizotinib, entrectinib, and lorlatinib) had weaker effects at the same concentration. This potency trend was confirmed by pathway analysis. Although taletrectinib was not evaluated in the colony formation assay, western blot analysis indicated that Compound 1 was more potent than taletrectinib in ROS1-driven NIH3T3 cells, consistent with observations in Example 7.

### Example 9: Cellular Phosphorylation Assay

Summary: The relative potency of Compound 1 and other ROS1 inhibitors between TRK and ROS1 was evaluated.

Method: For Ba/F3 TRKB cell phosphorylation assay, cells were seeded into 384-well plates, and test compounds were added in a 3-fold dilution series in full culture medium+10% FBS. Cells were stimulated with 100 ng/mL BDNF for 20 minutes. TRK phosphorylation was measured using the phospho-TRKA (Tyr674/675)/phospho-TRKB (Tyr706/Tyr707) AlphaLISA reagent (PerkinElmer #ALSU-PTRKAB). Untreated wells served as negative controls (no inhibition), whereas wells treated with high concentrations of the non-specific kinase inhibitor staurosporine served as positive controls (full inhibition). IC₅₀ was calculated from percent inhibition and inhibitor concentration using 4-parameter logistic regression.

NIH3T3 cells expressing EZR-ROS1 wild-type or mutant fusions were treated with the indicated concentrations of inhibitor for 3 hours prior to harvest. Cells were washed with PBS and harvested with cell lysis buffer supplemented with 0.25% deoxycholate, 0.05% SDS, and protease and phosphatase inhibitors. Protein concentrations were determined using the Pierce™ BCA Protein Assay (ThermoFisher Scientific). Lysates were extracted using Laemelli sample buffer supplemented with beta-mercaptoethanol for 10 minutes at 75° C. and lysates were run on 4-20% precast gradient Bis-tris gels (Invitrogen; ThermoFisher Scientific). Proteins were transferred to nitrocellulose membranes (Prometheus) and probed with phospho-ROS1 Y2274 (3078; 1:1000; Cell Signaling Technology), DYKDDDDK [Flag] (8H8L17; 1:1000; Invitrogen), phospho-SHP2 (A5278; 1:1000; Bimake), phospho-ERK1/2 (9101; 1:1000; Cell Signaling Technology), ERK2 (sc-1647; 1:1000; Santa Cruz), phospho-S6 (4858; 1:1000; Cell Signaling Technology), S6 (2216; 1:1000, Cell Signaling Technology), phospho-Akt (4060; 1:1000, Cell Signaling Technology), Akt (9272; 1:1000, Cell Signaling Technology), or Actin (JLA-20; 1:5000; Developmental Studies Hybridoma Bank). Signal was detected using HRP-conjugated or IR dye secondary antibodies on a BioRad ChemiDoc imaging station or a LI-COR Odyssey imaging station, respectively.

Results: TRK-related neurological toxicities are most consistent with the known functions of the TRKB signaling pathway. Accordingly, an assay was developed that uses Ba/F3 cells stably expressing full-length TRKB. Stimulation of this cell line with the brain-derived neurotrophic factor (BDNF) promotes TRKB autophosphorylation (pTRKB). Consistent with our biochemical and cell viability assays, Compound 1 only weakly inhibited cellular TRKB phosphorylation (IC₅₀=850 nM), affording a wide selectivity window for both wild-type ROS1 and ROS1 G2032R over pTRKB (670-fold and 240-fold) that was not achieved with any other ROS1 TKI tested.

IC₅₀ (nM) measurement of Compound 1 and several ROS1 inhibitors are shown below.

| Model name | Assay | Cmpd 1 | Crizotinib | Entrectinib | Lorlatinib | Taletrectinib | Repotrectinib |
|---|---|---|---|---|---|---|---|
| Ba/F3 CD74- | cell viability | 1.3 | 40 | 19 | 1.2 | 21 | 4.6 |
| ROS1 | | | | | | | |
| Ba/F3 CD74- | cell viability | 3.6 | 950 | 880 | 320 | 84 | 25 |
| ROS1 | | | | | | | |
| G2032R | | | | | | | |
| Ba/F3 TRKB | phosphorylation | 850 | 310 | 62 | 730 | 120 | <0.8 |

### Example 10: Preclinical Intracranial Activity

Method: All the procedures related to animal handling, care, and the treatment in these studies were performed according to guidelines approved by the IACUC of Pharmaron following the guidance of AAALAC.

Compound 1 was formulated as 1 mg/mL suspension in 20% hydroxypropyl-β-cyclodextrin (HP-β-CD) in deionized water. Compounds were administered orally to male Wistar Han rats (n=3 each). After 1 hour, brain samples and plasma were collected, and brain samples were homogenized in phosphate-buffered saline (PBS). Brain and plasma samples were precipitated by acetonitrile and centrifugation (4700 rpm, 15 minutes). Drug concentrations in the supernatants were quantified by LC/MS/MS. Unbound fractions were determined using rapid equilibrium dialysis. K_{p,uu} was calculated as the ratio between unbound drug concentration in the brain and unbound drug concentration in the plasma.

Ba/F3 CD74-ROS1 G2032R cells were transduced with viral particles containing the firefly luciferase gene and a neomycin-resistance marker. Infected cells were selected on neomycin, and monoclonal cultures were established through limiting dilution. Successful transformants were confirmed by Sanger sequencing and bioluminescence. For the in vivo study, 1 × 105 Ba/F3 CD74-ROS1 G2032R luciferase cells were stereotactically implanted into the right forebrains of 6- to 8-week-old female Balb/c nude mice. After 5 days, mice were randomized based on mean bioluminescence signal into 3 groups of n=7-10 mice each and received vehicle or Compound 1, 2 mg/kg, PO, BID. Bioluminescence and body weight were measured at regular intervals until the end of the study (61 days after treatment start) or until animals met the criteria for euthanasia.

Results: Compound 1 exhibited a favorable K_{p,uu} (unbound brain-to-plasma partitioning) of 0.16 measured at 1 hour after a single oral 10 mg/kg dose in Wistar-Han rats. To assess intracranial antitumor activity, a Ba/F3 CD74-ROS1 G2032R luciferase model was generated which permitted monitoring of the brain tumor burden via live-animal bioluminescence imaging. Ba/F3 CD74-ROS1 G2032R luciferase cells were intracranially injected in mice and rapid tumor growth was observed. All vehicle-treated mice lost body weight and succumbed to disease by day 19 with a median overall survival (mOS) of 16.5 days. In contrast, Compound 1 at 2 mg/kg suppressed intracranial tumor growth with no significant changes in body weight and all mice surviving to study termination (mOS >61 days), corresponding to a >4-fold survival extension over vehicle treatment. These results indicated that Compound 1 has strong intracranial antitumor activity.

### Example 11: In Vivo Pharmacology

Method: Mice bearing PDX tumors harboring SDC4-ROS1 were treated with a single dose (PO) of vehicle (20% HP-β-CD) or Compound 1 followed by tumor collection at the indicated timepoints (n=3) and western blot. Anti-β2-microglobulin (B2m) is a loading control that does not recognize mouse B2-microglobulin. pERK, pAKT, and cleaved PARP are markers of MAPK signaling, PI3K signaling and apoptosis, respectively.

Results: The results of this study are shown in FIG. 9. Direct inhibition of ROS1 fusions by Compound 1 in vivo was supported by downstream inhibition of signaling pathways MAPK and PI3K as measured by reductions in levels of phospho-p44/42 MAPK ERK1/2 (pERK) and phospho-AKT (pAKT), respectively. These results indicate that Compound 1 inhibits signaling through the MAPK and PI3K/AKT pathways and induces apoptosis in PDX tumors harboring SDC4-ROS1.

### Example 12: Cell Viability Assay for LTK Cell Lines

Generation of Ba/F3 Stable Cell Lines: Genes encoding CLIP1-LTK were synthesized, cloned into the retroviral construct pMSCV-puro (Biovector), and packaged into retroviral particles. The virus was used to infect Ba/F3 cells (RIKEN) at multiplicity of infection=1 or 10 for 1 day. Infected cells were rescued in media (RPMI-1640 with 10% fetal bovine serum and 1% streptomycin and penicillin) supplemented with mouse IL-3 (10 ng/mL) for 2 days, and stable cell lines were selected by IL-3 withdrawal and puromycin (0.8 μg/mL) for 7 days. Transformation of desired genes was confirmed by Sanger sequencing and western blot.

Cell Proliferation Assay: Stable cells were plated at 1,000 cells/well (40 μL) in a 384-well plate for 1 day. Test compounds (40 nL) were then added in a 3-fold dilution series using the TECAN EVO200 liquid handler and incubated for 72 hours. Plates were equilibrated at room temperature for 15 minutes followed by addition of 40 μL CellTiter-Glo reagent (Promega). Luminescence was measured on a plate reader. Half-maximal inhibitory concentration (IC₅₀) was calculated from percent inhibition and inhibitor concentration using four-parameter logistic regression.

Results: The IC₅₀ for Compound 1 against the Ba/F3 CLIP1-LTK cell line was determined to be 9.57 nM.

### Example 13: Ba/F3 Stable Cell Lines with L2086F mutation

### Biochemical Kinase Assay

ROS1 L2086F (amino acids 1881-2347) was custom-made at SignalChem (cat #NP72-092G/Y4232-2). Kinase reaction was initiated by mixing ATP (1 mM) with the fluorogenic phosphorylation substrate AQT0101 (15 μM, AssayQuant) and ROS1 L2086F (0.5 nM, SignalChem) in buffer (54 mM HEPES pH 7.5, 0.012% Brij-35, 0.52 mM EGTA, 1.2 mM DTT, 1% glycerol, 0.2 mg/mL BSA, 10 mM MgCl2). The plate was sealed and read by SpectraMax Paradigm at λ=485 nm every 2 minutes for 120 minutes at 30° C. Initial rates of reaction (v) were calculated from the change in fluorescence intensity over time during the initial, linear portion of the reaction. Finally, apparent inhibitory constants (Kᵢ^{app}) were determined from regression of v and I (inhibitor concentration) to Morrison Equation (E=enzyme concentration):

$$v = 1 - \frac{( E + I + {K}_{i}^{app} ) - \sqrt{{( E + I + {K}_{i}^{app} )}^{2} - 4 EI}}{2 E}$$

### Generation of Ba/F3 Stable Cell Lines

The gene encoding CD74-ROS1 L2086F was synthesized at GeneRay, cloned into the retroviral construct pMSCV-puro (Biovector), and packaged into retroviral particles. The virus was used to infect Ba/F3 cells (RIKEN) at multiplicity of infection=1 for 1 day. Infected cells were rescued in media (RPMI-1640 with 10% fetal bovine serum and 1% streptomycin and penicillin) supplemented with mouse IL-3 (10 ng/ml) for 2 days, and the stable cell line was selected by IL-3 withdrawal and puromycin (0.8 μg/mL) for 7 days. Transformation of the desired gene was confirmed by Sanger sequencing and western blot using the ROS1 antibody (CST #3287).

### Cell Proliferation Assay

Stable cells were plated at 1,000 cells/well (40 μL) in a 384-well plate for 1 day. Test compounds (40 nL) were then added in a 3-fold dilution series using the TECAN EVO200 liquid handler and incubated for 72 hours. Plates were equilibrated at room temperature for 15 minutes followed by addition of 40 μL CellTiter-Glo reagent (Promega). Luminescence was measured on a plate reader. Half-maximal inhibitory concentration (IC₅₀) was calculated from percent inhibition and inhibitor concentration using four-parameter logistic regression. The data of cell proliferation assay is summarized in the table below.

| | Crizotinib | Entrectinib | Lorlatinib | Taletrectinib | Repotrectinib | Compound 1 |
|---|---|---|---|---|---|---|
| IC₅₀ (nM) | 748 | 1.89E+03 | 8.00E+03 | 1.71E+03 | 1.24E+03 | 6.82E+03 |

A number of references have been cited, the disclosures of which are incorporated herein by reference in their entirety.

The embodiments described above are intended to be merely exemplary, and those skilled in the art will recognize, or will be able to ascertain using no more than routine experimentation, numerous equivalents of specific compounds, materials, and procedures. All such equivalents are considered to be within the scope of the invention and are encompassed by the appended claims.

What is claimed is:

1. A method of treating a patient with solid tumor, comprising administering to said patient a therapeutically effective amount of Compound 1: 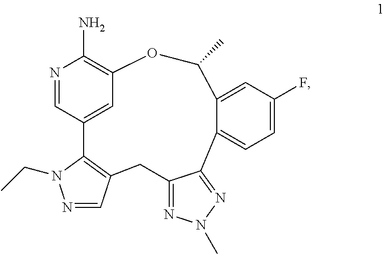 [SMILES: CCn1ncc2c1-c1cnc(N)c(c1)O[C@H](C)c1cc(F)ccc1-c1nn(C)nc1C2]
or a stereoisomer, or a mixture of stereoisomers thereof, or a pharmaceutically acceptable salt thereof.

2. The method of claim 1, wherein the solid tumor is advanced solid tumor.

3. The method of claim 2, wherein the advanced solid tumor is relapsed after, refractory to, or resistant to the prior treatment by a tyrosine kinase inhibitor (TKI).

4. The method of any one of claims 1 to 3, wherein the solid tumor is non-small cell lung cancer (NSCLC).

5. The method of any one of claims 1 to 4, wherein the solid tumor is metastatic.

6. The method of claim 5, wherein the solid tumor is CNS metastatic.

7. The method of any one of claims 1 to 6, wherein the solid tumor is ROS1 positive.

8. The method of any one of claims 1 to 7, wherein the solid tumor has a ROS1 mutation.

9. The method of claim 8, wherein the ROS1 mutation is G2032R.

10. The method of any one of claims 1 to 9, wherein the solid tumor has a ROS1 fusion.

11. The method of any one of claims 1 to 6, wherein the solid tumor is ALK positive (e.g. ALK fusion).

12. The method of any one of claims 1 to 6, wherein the solid tumor is LTK positive.

13. The method of any one of claims 1 to 12, wherein the patient is naïve to tyrosine kinase inhibitor (TKI) therapy.

14. The method of any one of claims 1 to 12, wherein the patient has been treated with one prior TKI therapy.

15. The method of any one of claims 1 to 12, wherein the patient has been treated with at least one prior TKI therapy.

16. The method of any one of claims 1 to 12, wherein the patient has been treated with at least two prior TKI therapies.

17. The method of any one of claims 13 to 16, wherein the TKI is ROS1 TKI.

18. The method of any one of claims 14 to 17, wherein the prior TKI therapy is one or more selected from the group consisting of crizotinib, ceritinib, alectinib, brigatinib, lorlatinib, entrectinib, repotrectinib, cabozantinib, foretinib, merestinib, taletrectinib, masitinib, or ensartinib.

19. The method of any one of claims 1 to 18, wherein the patient has not been treated with prior platinum-based chemotherapy.

20. The method of any one of claims 1 to 18, wherein the patient has been treated with up to one prior platinum-based chemotherapy.

21. The method of any one of claims 1 to 18, wherein the patient has been treated with one prior platinum-based chemotherapy.

22. The method of any one of claims 1 to 21, wherein the patient has not been treated with immunotherapy.

23. The method of any one of claims 1 to 21, wherein the patient has been treated with immunotherapy.

24. The method of any one of claims 1 to 23, wherein the patient has not been treated with chemotherapy.

25. The method of any one of claims 1 to 23, wherein the patient has been treated with at least one prior line of chemotherapy.

26. The method of any one of claims 1 to 23, wherein the patient has been treated with at least two prior lines of chemotherapy.

27. The method of any one of claims 1 to 26, wherein the patient has been treated with at least three prior lines of anticancer therapy.

28. The method of any one of claims 1 to 27, wherein the patient has been treated with at least two prior lines of anticancer therapy selected from the group consisting of ROS1 TKI (e.g., investigational ROS1 TKI, crizotinib, lorlatinib, entrectinib, repotrectinib, and taletrectinib) and chemotherapy.

29. The method of any one of claims 1 to 28, wherein the patient has been treated with at least one line of ROS1 TKI and one line of chemotherapy.

30. The method of any one of claims 1 to 29, wherein the patient has been treated with at least two lines of ROS1 TKI and one line of chemotherapy.

31. The method of any one of claims 1 to 30, wherein the patient has been treated with at least three lines of ROS1 TKI and one line of chemotherapy.

32. The method of any one of claims 1 to 31, wherein the patient has been treated with at least two lines of chemotherapy.

33. The method of any one of claims 1 to 32, wherein the patient has been treated with at least one line of ROS1 TKI and two lines of chemotherapy.

34. The method of any one of claims 1 to 33, wherein the patient has been treated with at least two lines of ROS1 TKI and two lines of chemotherapy.

35. The method of any one of claims 1 to 34, wherein the patient has been treated with at least three lines of ROS1 TKI and two lines of chemotherapy.

36. The method of any one of claims 17 to 35, wherein the ROS1 TKI is crizotinib.

37. The method of any one of claims 17 to 35, wherein the ROS1 TKI is entrectinib.

38. The method of any one of claims 17 to 35, wherein the ROS1 TKI is lorlatinib.

39. The method of any one of claims 17 to 35, wherein the ROS1 TKI is repotrectinib.

40. The method of any one of claims 1 to 39, wherein the patient has been treated with lorlatinib and repotrectinib.

41. The method of any one of claims 1 to 40, wherein the solid tumor is advanced or metastatic ROS1-positive solid tumor, and the patient has been treated with at least one prior ROS1 TKI therapy.

42. The method of any one of claims 1 to 40, wherein the solid tumor is advanced or metastatic ROS1-positive NSCLC, and the patient is naïve to TKI therapy and has been treated with up to one prior platinum-based chemotherapy with or without immunotherapy.

43. The method of any one of claims 1 to 40, wherein the solid tumor is advanced or metastatic ROS1-positive NSCLC, and the patient has been treated with one prior ROS1 TKI therapy and has not been treated with prior platinum-based chemotherapy or immunotherapy.

44. The method of any one of claims 1 to 40, wherein the solid tumor is advanced or metastatic ROS1-positive NSCLC, and the patient has been treated with one prior ROS1 TKI therapy and one prior platinum-based chemotherapy with or without immunotherapy.

45. The method of any one of claims 1 to 40, wherein the solid tumor is advanced or metastatic ROS1-positive NSCLC, and the patient has been treated with at least two prior ROS1 TKI therapies and up to one prior platinum-based chemotherapy with or without immunotherapy.

46. The method of any one of claims 1 to 45, wherein the solid tumor is advanced or metastatic ROS1-positive solid tumor, and the patient has progressed on a prior therapy.

47. The method of any one of claims 1 to 46, wherein Compound 1 is administered to the patient for one or more days.

48. The method of any one of claims 1 to 47, wherein Compound 1 is administered to the patient for at least one treatment cycle.

49. The method of claim 48, wherein one treatment cycle is at least 7 days.

50. The method of claim 48, wherein one treatment cycle is at least 14 days.

51. The method of claim 48, wherein one treatment cycle is at least 21 days.

52. The method of claim 48, wherein one treatment cycle is at least 28 days.

53. The method of any one of claims 1 to 52, wherein the patient does not experience a Grade 4 adverse event (e.g., TRAE) after the administration of Compound 1.

54. The method of any one of claims 1 to 53, wherein the patient does not experience a Grade 3 adverse event (e.g., TRAE) after the administration of Compound 1.

55. The method of any one of claims 1 to 54, wherein the patient does not experience a Grade 2 adverse event (e.g., TRAE) after the administration of Compound 1.

56. The method of any one of claims 1 to 55, wherein the patient does not experience a Grade 1 adverse event (e.g., TRAE) after the administration of Compound 1.

57. The method of any one of claims 1 to 55, wherein the patient experiences at most a Grade 1 adverse event (e.g., TRAE) after the administration of Compound 1.

58. The method of claim 57, wherein the Grade 1 adverse event is fatigue, myalgia or nausea.

59. The method of any one of claims 1 to 58, wherein the patient only experiences nausea after the administration of Compound 1.

60. The method of any one of claims 1 to 59, wherein the patient does not experience a CNS adverse event after the administration of Compound 1.

61. The method of claim 60, wherein the CNS adverse event is one or more selected from the group consisting of dizziness, ataxia, gait disturbance, paraesthesia, weight gain, hyperphagia, paresthesias, abnormal movement, cognitive changes, speech effects (e.g, dysarthria, slow speech, or speech disorder), mood disorder (e.g., irritability, anxiety, depression, affect lability, personality change, mood swings, affective disorder, aggression, agitation, mood altered, depressed mood, euphoric mood, or mania), and cognitive disorder (e.g., memory impairment, cognitive disorder, amnesia, confusion, disturbance in attention, delirium, mental impairment, attention deficit/hyperactivity disorder, dementia, sleep disturbance, or reading disorder).

62. The method of any one of claims 1 to 61, wherein the patient does not experience an adverse event of weight gain and/or glucose metabolism disorders.

63. The method of any one of claims 1 to 62, wherein the patient is a patient population.

64. The method of claim 63, wherein less than 30% of the patient population experiences a Grade 1 TRAE after administration of Compound 1.

65. The method of claim 63, wherein less than 20% of the patient population experiences a Grade 2 TRAE after administration of Compound 1.

66. The method of claim 63, wherein the patient population experiences no Grade 3 or Grade 4 TRAE after administration of Compound 1.

67. The method of any one of claims 1 to 66, wherein the patient has a complete response after one or more cycles of treatment.

68. The method of any one of claims 1 to 66, wherein the patient has a partial response after one or more cycles of treatment.

69. The method of any one of claims 1 to 68, wherein the patient has reached stable disease after one or more cycles of treatment.

70. The method of any one of claims 1 to 69, wherein the patient has brain metastases.

71. The method of any one of claims 1 to 70, wherein the patient has brain metastases and experiences no intracranial progression after at least one treatment cycle.

72. The method of any one of claims 1 to 71, wherein the patient has at least about 5% to about 100% reduction of ROS1 allele variant in circulating tumor DNA after at least one treatment cycle.

73. The method of any one of claims 1 to 72, wherein the patient has at least about 35% reduction of ROS1 allele variant in circulating tumor DNA after at least one treatment cycle.

74. The method of any one of claims 1 to 73, wherein the patient has at least about 55% reduction of ROS1 allele variant in circulating tumor DNA after at least one treatment cycle.

75. The method of any one of claims 1 to 74, wherein the patient has at least about 65% reduction of ROS1 allele variant in circulating tumor DNA after at least one treatment cycle.

76. The method of any one of claims 1 to 75, wherein the patient has at least about 100% reduction of ROS1 allele variant in circulating tumor DNA after at least one treatment cycle.

77. The method of any one of claims 1 to 76, wherein the patient has undetectable ROS1 allele variant in circulating tumor DNA after at least one treatment cycle.

78. The method of claim 77, wherein the ROS1 allele variant is G2032R.

79. The method of any one of claims 46 to 78, wherein the prior therapy is a prior ROS1 TKI therapy.

80. The method of claim 79, wherein the ROS1 TKI is crizotinib, entrectinib, taletrectinib, lorlatinib, or repotrectinib.

81. The method of any one of claims 1 to 80, wherein administration of Compound 1 provides the area under the curve from 0 to 24 hours (AUC₀₋₂₄) of the compound in a range of (80% to 125% of 500 ng*h/mL) to (80% to 125% of 30000 ng*h/mL).

82. The method of any one of claims 1 to 81, wherein administration of Compound 1 provides the area under the curve from 0 to 24 hours (AUC₀₋₂₄) of the compound in a range of about 500 to about 30000 ng*h/mL.

83. The method of any one of claims 1 to 82, wherein the area under the curve from 0 to 24 hours (AUC₀₋₂₄) of the compound is in a range of about 1000 to about 13000 ng*h/mL after about half cycle of treatment.

84. The method of any one of claims 1 to 83, wherein such administration provides the area under the curve from 0 to 24 hours after administration (AUC₀₋₂₄) of the compound in a range of (80% to 125% of 20 ng*h/mL) to (80% to 125% of 500 ng*h/mL) for every mg of Compound 1 administered.

85. The method of any one of claims 1 to 84, wherein such administration provides the area under the curve from 0 to 24 hours after administration (AUC₀₋₂₄) of the compound in a range of from about 20 to about 500 ng*h/mL for every mg of Compound 1 administered.

86. The method of any one of claims 1 to 85, wherein such administration provides the area under the curve from 0 to 24 hours after administration (AUC₀₋₂₄) of the compound in a range of from about 50 to about 200 ng*h/mL after about half cycle of treatment.

87. The method of any one of claims 1 to 86, wherein such administration provides the AUC_{last,unbound} of the compound in a range of about 400 to about 7000 ng*h/mL.

88. The method of any one of claims 1 to 87, wherein such administration provides the AUC_{last,unbound} of the compound in a range of from about 10 to about 80 ng*h/mL for every mg of Compound 1 administered.

89. The method of any one of claims 1 to 88, wherein such administration provides the AUCₜₐᵤ of the compound in a range of about 2000 to about 8000 ng*h/mL.

90. The method of any one of claims 1 to 89, wherein such administration provides the AUCₜₐᵤ of the compound in a range of about 50 to about 150 ng*h/mL for every mg of Compound 1 administered.

91. The method of any one of claims 1 to 90, wherein such administration provides the AUC_{inf} of the compound in a range of about 4000 to about 15000 ng*h/mL.

92. The method of any one of claims 1 to 91, wherein such administration provides the AUC_{inf} of the compound in a range of about 50 to about 250 ng*h/mL for every mg of Compound 1 administered.

93. The method of any one of claims 1 to 92, wherein such administration provides the maximum plasma concentration (Cₘₐₓ) of the compound in a range of (80% to 125% of 100 ng/mL) to (80% to 125% of 1500 ng/mL).

94. The method of any one of claims 1 to 93, wherein such administration provides the maximum plasma concentration (Cₘₐₓ) of the compound in a range of about 100 to about 1500 ng/mL.

95. The method of any one of claims 1 to 94, wherein such administration provides the maximum plasma concentration (Cₘₐₓ) of the compound in a range of about 2 to about 50 ng/mL for every mg of Compound 1 administered.

96. The method of any one of claims 1 to 95, wherein such administration provides the minimum plasma concentration reached by Compound 1 during the time interval between administration of two doses (Cₘᵢₙ) in a range of about 50 to about 350 ng/ml.

97. The method of any one of claims 1 to 96, wherein such administration provides the unbound maximum plasma concentration (C_{max,unbound}) of the compound in a range of about 30 ng/ml to about 400 ng/ml.

98. The method of any one of claims 1 to 97, wherein such administration provides the Tₘₐₓ of the compound after the administration in a range of about 0.25h to about 5h, or about 0.7h to about 1.3h.

99. The method of any one of claims 1 to 98, wherein such administration provides the t_{1/2} of the compound after the administration in a range of about 2h to about 50h, or about 10h to about 24h.

100. The method of any one of claims 1 to 99, wherein the administration provides the dose plasma concentration of the compound at least 10%, 20%, 30%, 40%, or 50% higher than the IC₅₀ of the compound against ROS1 G2032R mutant during at least 70%, 80%, 90%, 95%, 97%, 98%, or 99% of about 24 hours immediately following the administration.

101. A method of reducing a lesion in a subject having a ROS1-positive solid tumor, comprising
(i) obtaining a first radiological measurement of the size of the lesion;
(ii) administering a pharmaceutically effective amount of Compound 1 once daily for one or more days; and
(iii) obtaining a second radiological measurement of the size of the lesion;
wherein the second measurement is at most 100% of the first measurement.

102. The method of claim 101, wherein the second measurement is at most about 90% of the first measurement.

103. The method of claim 101, wherein the second measurement is at most about 80% of the first measurement.

104. The method of claim 101, wherein the second measurement is at most about 70% of the first measurement.

105. The method of claim 101, wherein the second measurement is at most about 60% of the first measurement.

106. The method of claim 101, wherein the second measurement is at most about 50% of the first measurement.

107. The method of claim 101, wherein the second measurement is about 0.01% to about 90% of the first measurement.

108. The method of claim 101, wherein the second measurement shows no detectable lesion.

109. The method of any one of claims 1 to 108, wherein the compound is administered at an amount of from about 5 mg to about 500 mg (by weight of Compound 1 free base) once daily.

110. The method of claim 109, wherein the compound is administered at an amount of from about 25 mg to about 250 mg (by weight of free base Compound 1) once daily.

111. The method of claim 109, wherein the compound is administered at an amount of from about 25 mg to about 200 mg (by weight of free base Compound 1) once daily.

112. The method of claim 109, wherein the compound is administered at an amount of about 50 mg (by weight of free base Compound 1) once daily.

113. The method of claim 109, wherein the compound is administered at an amount of about 75 mg (by weight of free base Compound 1) once daily.

114. The method of claim 109, wherein the compound is administered at an amount of about 100 mg (by weight of free base Compound 1) once daily.

115. The method of claim 109, wherein the compound is administered at an amount of about 125 mg (by weight of free base Compound 1) once daily.

116. The method of claim 109, wherein the compound is administered at an amount of about 5 mg, about 10 mg, about 15 mg, about 20 mg, about 25 mg, about 30 mg, about 35 mg, about 40 mg, about 45 mg, about 50 mg, about 75 mg, about 100 mg, about 125 mg, or about 150 mg (by weight of free base Compound 1) once daily.

117. The method of any one of claims 1 to 108, wherein the compound is administered at an amount of from about 5 mg to about 500 mg (by weight of Compound 1 free base) twice daily (BID).

118. The method of claim 117, wherein the compound is administered at an amount of from about 25 mg to about 250 mg (by weight of free base Compound 1) twice daily (BID).

119. The method of claim 117, wherein the compound is administered at an amount of from about 25 mg to about 200 mg (by weight of free base Compound 1) twice daily (BID).

120. The method of claim 117, wherein the compound is administered at an amount of from about 25 mg to about 150 mg (by weight of free base Compound 1) twice daily (BID).

121. The method of claim 117, wherein the compound is administered at an amount of from about 25 mg to about 100 mg (by weight of free base Compound 1) twice daily (BID).

122. The method of claim 117, wherein the compound is administered at an amount of about 5 mg, about 10 mg, about 15 mg, about 20 mg, about 25 mg, about 30 mg, about 35 mg, about 40 mg, about 45 mg, about 50 mg, about 75 mg, about 100 mg, about 125 mg, or about 150 mg (by weight of free base Compound 1) twice daily (BID).

123. The method of any one of claims 1 to 122, wherein the compound is administered orally.

124. The method of claim 123, wherein the compound is administered in the form of one or more tablets.

125. The method of claim 124, wherein the tablet has a unit dose strength of about 5 mg or about 25 mg by weight of free base Compound 1.

126. The method of any one of claims 1 to 125, wherein the compound is administered to a patient at fasted status.

127. The method of any one of claims 1 to 125, wherein the compound is administered to a patient at fed status.

128. The method of any one of claims 1 to 127, wherein the patient is not taking any one of strong inhibitors of CYP3A4, strong inducers of CYP3A4, sensitive substrates of CYP3A4, substrates of P-gp/multidrug resistance protein (MDR1), substrates of BCRP/breast cancer resistance protein (ABCG2), substrates of MATE1, or gastric acid reducing agents.

129. The method of any one of claims 1 to 127, wherein the patient is taking any one of strong inhibitors of CYP3A4, strong inducers of CYP3A4, sensitive substrates of CYP3A4, substrates of P-gp/multidrug resistance protein (MDR1), substrates of BCRP/breast cancer resistance protein (ABCG2), substrates of MATE1, or gastric acid reducing agents.

130. The method of any one of claims 1 to 127, wherein the compound is administered in the absence of a sensitive substrate of CYP3A4.

131. The method of claim 130, wherein the sensitive substrates of CYP3A4 comprise one or more of buspirone, everolimus, lovastatin, midazolam, simvastatin, triazolam, maraviroc, conivaptan, and darifenacin.

132. The method of any one of claims 1 to 131, wherein the patient does not experience one or more symptoms selected from the group consisting of cognitive impairment, mood disorders, sleep disturbances, dizziness, ataxia, and weight gain, after the administration of the compound.

133. The method of any one of claims 1 to 132, wherein the patient experiences reduced levels of one or more of pROS1, ROS1, pAKT, and pERK, after the administration of the compound.

134. The method of any one of claims 1 to 133, wherein the patient experiences reduced expression level of one or more MAP kinase pathway genes in tumor, after the administration of the compound.

135. The method of any one of claims 1 to 134, wherein the patient experiences reduced expression level of one or more MAP kinase pathway genes in solid tumor, after the administration of the compound.

136. The method of claim 134 or 135, wherein the one or more MAP kinase pathway genes are selected from the group consisting of DUSP6, FOS, ILIR1, and SPRY4.

137. The method of any one of claims 1 to 136, wherein the compound is Compound 1 free base.

138. The method of any one of claims 1 to 137, wherein the compound administered is a solid form of Compound 1 characterized by an XRPD pattern comprising peaks at approximately 10.7, 15.0, and 21.2° 2θ) (+0.2°.

139. The method of any one of claims 1 to 138, wherein the compound is administered as a pharmaceutical composition comprising Compound 1, or a stereoisomer, or a mixture of stereoisomers thereof, or a pharmaceutically acceptable salt thereof, and a diluent, a binder, a disintegrant, and a lubricant.

140. The method of claim 139, wherein the diluent is mannitol, the binder is a mixture of HPC and SMCC, the disintegrant is sodium starch glycolate (SSG), and the lubricant is magnesium stearate.
